(19) United States
(12) Patent Application Publication
(10) Pub. No.: US 2022/0168402 A1
(43) Pub. Date: Jun. 2, 2022

(54) PIP4Ks SUPPRESS INSULIN SIGNALING AND ENHANCE IMMUNE FUNCTION THROUGH A CATALYTIC-INDEPENDENT MECHANISM

(71) Applicant: Cornell University, Ithaca, NY (US)

(72) Inventors: Lewis C. Cantley, Cambridge, MA (US); Marcia Noreen Paddock, New York, NY (US); Diana Grace Wang, New York, NY (US)

(21) Appl. No.: 17/602,540

(22) PCT Filed: Apr. 10, 2020
(86) PCT No.: PCT/US2020/027745

§ 371 Date: Oct. 8, 2021

Related U.S. Application Data
(60) Provisional application No. 62/831,895, filed on Apr. 10, 2019.
(60) Provisional application No. 62/847,411, filed on May 14, 2019.

(51) Int. Cl. A61K 38/45; A61K 31/713; A61P 3/10; A61P 37/02; C12N 9/12; A61K 9/127

(52) U.S. Cl. CPC A61K 38/45; A61K 31/713; A61P 3/10; C12Y 207/01149; C12N 9/1205; A61K 9/127; A61P 37/02

(57) ABSTRACT

As described herein, phosphatidylinositol-5-phosphate 4-kinases (PIP4Ks) have an allosteric function in addition to their catalytic activity. The allosteric function suppresses PIP5K-mediated PI(4,5)P₂ synthesis and insulin-dependent conversion to PI(3,4,5)P₃. Further described herein are methods for treatment of diabetes, metabolic syndrome, insulin resistance, a obesity, cancer, immune deficiency, autoimmune disease, infection, or a combination thereof.

This application claims benefit of priority to the filing date of U.S. Provisional Application Ser. No. 62/831,895, filed Apr. 10, 2019, and to the filing date of U.S. Provisional Application Ser. No. 62/847,411, filed May 14, 2019, the contents of which applications are specifically incorporated by reference herein in their entireties.

### GOVERNMENT FUNDING

This invention was made with government support under R35 CA197588, U54CA210184 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Apr. 9, 2020, is named 2029260.txt and is 81,920 bytes in size.

### BACKGROUND

Phosphatidylinositol-4,5-bisphosphate (PI(4,5)P₂) plays numerous roles in cellular regulation. It mediates actin remodeling at the plasma membrane, modulates vesicle trafficking, and is the substrate that hormone-stimulated phospholipases type C (PLC) utilize to generate the second messengers diacylglycerol and inositol-1,4,5-trisphosphate (Balla, Physiol Rev 93: 1019-1137 (2013); Sun et al., Bioessays 35: 513-522 (2013). PI(4,5)P₂ is also the substrate that Class 1 phosphoinositide 3-kinases use (Saito et al., Immunity 19: 669-678 (2003)) to generate the second messenger phosphatidylinositol-3,4,5-trisphosphate (PI(3,4,5)P₃) in response to insulin and other growth factors (Fruman et al., Cell 170: 605-635 (2017)).

Yeast have a single enzyme for generating PI(4,5)P₂ encoded by MSS4 while mammals have six genes encoding enzymes that generate PI(4,5)P₂. PIP5K1A, PIP5K1B and PIP5K1C produce PI(4,5)P₂ from phosphatidylinositol-4-phosphate (PI(4)P), while PIP4K2A, PIP4K2B and PIP4K2C generate PI(4,5)P₂ from phosphatidylinositol-5-phosphate (PI(5)P): (Rameh et al., Nature 390: 192-196 (1997; van den Bout & Divecha, J Cell Sci 122: 3837-3850 (2009)).

Speculation exists that the function of the PIP4Ks is primarily to decrease the level of PI(5)P (Jones et al., Mol Cell 23: 685-695 (2006); Wilcox & Hinchliffe, FEBS Lett 582, 1391-1394 (2008)). PIP4K family members may also suppress insulin/PI3K/mTORC1 signaling in vivo, despite their enzymatic function to synthesize PI(4,5)P₂. Homozygous germline deletion of Pip4k2b^{-/-} in mice causes reduced adiposity and increased insulin sensitivity in muscle (Lamia et al., Mol Cell Biol 24, 5080-5087 (2004)), and Pip4k2c^{-/-} mice have enhanced TORC1 signaling (Shim et al., 2016). Additionally, the accumulation of PI(5)P, resulting from loss of the highly active forms of PIP4K, increases signaling through the PI3K pathway (Carricaburu et al., Proc Natl Acad Sci USA 100: 9867-9872 (2003); Pendaries et al., EMBO J 25: 1024-2034 (2006)).

### SUMMARY

Although PIP4K proteins appear to be involved in various cellular activities, multiple efforts to develop agents that successfully modulate PIP4K expression and function for therapeutic purposes have not been successful. For example, catalytic site inhibitors of PIP4K have been attempted, but as shown herein, catalytic site inhibitors do not cause changes in PI3K signaling.

As described herein, PIP4Ks have a catalytic-independent role in regulating PI3K signaling in cells and in downstream signaling pathways (AKT, PRAS40) that activate immune cell activation pathways. Although PIP4Ks can catalyze the production of phosphatidylinositol-4,5-bisphosphate (PI(4,5)P₂) from phosphatidylinositol-5-phosphate (PI(5)P), PIP4Ks also have a structural or scaffolding role that impacts insulin sensitivity, adiposity, and immune responses. For example, PIP4Ks can interact with or scaffold with other PIP4Ks and phosphatidylinositol-4-phosphate 5-kinases (PIP5Ks).

Provided herein are methods and compositions for degrading, modifying, or inhibiting one or more isoforms of phosphatidylinositol-5-phosphate 4-kinase (PIP4K). Such methods and compositions can enhance insulin signaling, reduce insulin resistance, and/or enhance immune functions in the subject.

The phosphatidylinositol-5-phosphate 4-kinase(s) so degraded, modified, or inhibited can be PIP4K2A, PIP4K2B, PIP4K2C, or a combination thereof.

### DESCRIPTION OF THE FIGURES

FIGS. 1A-1O illustrate validation of tools to eliminate all three phosphatidylinositol 5-phosphate 4-kinase (PIP4K) isoforms reveals paradoxical increase in phosphatidylinositol-4,5-bisphosphate (PI(4,5)P₂). FIG. 1A shows images of western blots illustrating the efficiency of knockdown of PIP4K isoforms in Hela cells. Cell line notation and their descriptions are listed in Table 4. FIG. 1B shows images of western blots illustrating PIP4K isoform expression in 293T clones with CRISPR mediated knockout of PIP4K2A, PIP4K2B, and PIP4K2C. The cell line descriptions are shown in Table 4. The parental cell line is in first lane and the CRISPR clonal cell line with intact PIP4K is designed as WT. A faint non-specific band that runs slightly faster than PIP4Ks is just visible. FIG. 1C graphically illustrates levels of phosphatidylinositol-5-phosphate (PI(5)P) as quantified by HPLC showing increases in PI(5)P within cells that have reduced PIP4K in PIP4K2A/B/C triple knockdown (TKD1 or TKD2, see Table 4) Hela cells. FIG. 1D graphically illustrates levels of phosphatidylinositol-5-phosphate (PI(5)P) as quantified by HPLC showing increases in PI(5)P within cells that have reduced PIP4K in PIP4K2A/B/C triple knockout (TKO1 or TKO2, see Table 4) 293T cells. FIG. 1E shows an image of a western blot, illustrating that knockdown of PIP4K enhances insulin signaling in Hela cells. Quantification of the pAkt-473 bands by Licor is indicated below the respective lanes. FIG. 1F illustrates immunofluorescent detection of lysosomal marker Lamp1 illustrating increased lysosomal accumulation of Lamp1 in 293T TKO versus control cells. Representative images are shown. FIG. 1G illustrates PI(4,5)P₂ increases in cells with loss of PIP4K, as shown in Hela TKD cells with measurement by HPLC. FIG. 1H illustrates that PI(4,5)P₂ increases in cells with loss of PIP4K, as shown in 293T TKO cells with measurement by HPLC. FIG. 1I illustrates that PI(4)P decreases in Hela TKD cells, which exhibit loss of PIP4K, as measured by HPLC. FIG. 1J illustrates that PI(4)P decreases in 293T TKO cells with reduced PIP4K, as measured by HPLC. FIG. 1K illustrates that 293T TKO cells have higher levels of PI(3,4,5)P₃ as measured by HPLC. Significance was calculated using ANOVA with Holm-Sidak multiple comparisons to control cell line with intact PIP4K. **p<0.01, ***p<0.001, ****p<0.0001. Data are represented as mean±SEM, n=3. FIGS. 1L-1O illustrate knockdown of multiple PIP4K isoforms. FIG. 1L is a schematic diagram illustrating miRE expression cassettes in doxycycline inducible vectors (LG3GEPIR, LG3REPIR) or constitutive vectors (SGEP, SREN). Hairpin expression can be concatemerized with GFP or infrared RFP (iRFP). FIG. 1M illustrates the kinetics of shRNA mediated knockdown of all three isoforms of PIP4K, with cell line descriptions in Table 4. 293T cells expressing doxycycline-inducible hairpins were harvested over the course of five days of doxycycline treatment and isoforms of PIP4K were detected. FIG. 1N illustrates validation of knockdown of PIP4K isoforms in 293T cells. Cell line descriptions are provided in Table 4. FIG. 1O graphically illustrates cell proliferation over 72 hours, graphed as mean confluence with +/−SEM (N=6). Cell line descriptions are provided in Table 4.

FIG. 2A-2Z illustrate identification of a catalytic-independent function of PIP4K. FIG. 2A graphically illustrates PI(4)P and PI(4,5)P₂ levels as detected by HPLC upon knockdown of one or multiple isoforms of PIP4K in 293T cells using tandem shRNA constructs. Hairpin constructs are indicated on x-axis, with descriptions in Tables 3-4. FIG. 2B graphically illustrates PI(4)P and PI(4,5)P₂ levels as detected by HPLC upon knockdown of one or multiple isoforms of PIP4K in Hela cells using tandem shRNA constructs. Hairpin constructs are indicated on x-axis, with descriptions in Tables 3-4. FIG. 2C shows a Western blot providing validation of rescue cell line panel with reconstitution of active or kinase dead PIP4K2A^{KD} or active PIP4K2C into Hela TKD cells at near endogenous levels. PIP4K tagged with three hemagglutinin tags (3×HA-PIP4K proteins) are larger than the endogenous protein. A faint non-specific band is visible running slightly faster than the endogenous protein. FIG. 2D shows a Western blot providing validation of rescue cell line panel with reconstitution of active or kinase dead PIP4K2A^{KD} or active PIP4K2C into 293T TKO cells at near endogenous levels. Tagged 3×HA-PIP4K proteins are larger than the endogenous protein. A faint non-specific band is visible running slightly faster than the endogenous protein. FIG. 2E graphically illustrates PI(4,5)P₂ levels in Hela TKD rescue cell lines as assessed by HPLC. FIG. 2F graphically illustrates PI(4,5)P₂ levels in 293T TKO rescue cell lines as assessed by HPLC. FIG. 2G graphically illustrates PI(4)P levels in Hela TKD rescue cell lines as assessed by HPLC. FIG. 2H graphically illustrates PI(4)P levels in 293T TKO rescue cell lines. FIG. 2I graphically illustrates PI(5)P levels in Hela TKD rescue cell lines. FIG. 2J graphically illustrates PI(5)P levels in 293T TKO rescue cell lines. For FIGS. 2E-2J significance was calculated using ANOVA with Holm-Sidak multiple comparisons to TKD or TKO cell lines. *p<0.05, **p<0.01, ***p<0.001, ****p<0.0001, and data are represented as mean±SEM, n=3. FIG. 2K-2Z illustrate depletion of PIP4K in a panel of cell lines of various tissue and mutational backgrounds. FIG. 2K illustrates knockdown (TDK) of PIP4K2A, PIP4K2B and PIP4K2C in the PaTu 8988t cell line with mutations as reported by the Cancer Cell Line Encyclopedia. FIG. 2L illustrates knockdown (TDK) of PIP4K2A, PIP4K2B and PIP4K2C in the H1299 cell line with mutations as reported by the Cancer Cell Line Encyclopedia. FIG. 2M illustrates knockdown (TDK) of PIP4K2A, PIP4K2B and PIP4K2C in the H1975 cell line with mutations as reported by the Cancer Cell Line Encyclopedia. FIG. 2N illustrates knockdown (TDK) of PIP4K2A, PIP4K2B and PIP4K2C in the MCF7 cell line with mutations as reported by the Cancer Cell Line Encyclopedia. FIG. 2O illustrates knockdown (TDK) of PIP4K2A, PIP4K2B and PIP4K2C in BJ immortalized fibroblasts. FIG. 2P illustrates expression of PI(4,5)P₂/PI in the PaTu 8988t cell line with mutations as reported by the Cancer Cell Line Encyclopedia. FIG. 2Q illustrates expression of PI(4)P/PI in the PaTu 8988t cell line with mutations as reported by the Cancer Cell Line Encyclopedia. FIG. 2R illustrates expression of PI(4,5)P₂/PI in the H1299 cell line with mutations as reported by the Cancer Cell Line Encyclopedia. FIG. 2S illustrates expression of PI(4)P/PI in the H1299 cell line with mutations as reported by the Cancer Cell Line Encyclopedia. FIG. 2T illustrates expression of PI(4,5)P2/PI in the H1975 cell line with mutations as reported by the Cancer Cell Line Encyclopedia. FIG. 2U illustrates expression of PI(4)P/PI in the H1975 cell line with mutations as reported by the Cancer Cell Line Encyclopedia. FIG. 2V illustrates expression of PI(4,5)P₂/PI in the MCF7 cell line with mutations as reported by the Cancer Cell Line Encyclopedia. FIG. 2W illustrates expression of PI(4)P/PI in the MCF7 cell line with mutations as reported by the Cancer Cell Line Encyclopedia. FIG. 2X illustrates expression of PI(4,5)P₂/PI in BJ immortalized fibroblasts. FIG. 2Y illustrates expression of PI(4)P/PI in BJ immortalized fibroblasts. FIG. 2Z graphically illustrates PI(4)P/PI to PI(4,5)P₂/PI ratios in different cell lines.

FIG. 3A-3Q illustrates that PIP4K inhibits PIP5K activity. FIG. 3A graphically illustrates changes in PIP5K activity in 293T cells with TKO genotypes. Cell pellets were normalized to cell number and sonicated in the presence of excess PI(4)P. Radioactive ATP³² was added for kinase reaction. Lipids were extracted and quantified by TLC with confirmation by HPLC after diacylation. FIG. 3B graphically illustrates quantification of in vitro kinase assay measuring conversion of PI(4)P to radiolabeled PI(4,5)P₂ by PIP5K1A. PIP5K1A activity is inhibited in vitro by addition of 2-10 fold molar excess of PIP4K2A, PIP4K2B, or PIP4K2C. FIG. 3C graphically illustrates that once denatured. PIP4K2C no longer inhibits PIP5K1A. FIG. 3D graphically illustrates that in the presence of 0.1% Triton-X100, PIP4K2A no longer inhibits PIP5K1A activity. FIG. 3E shows a western blot providing validation of near-endogenous level expression of 3×HA-PIP5K1A levels in 293T control and PIP5K1A TKO cells. Tagged 3×HA-PIP5K1A is slightly larger than the endogenous protein. FIG. 3F illustrates pulldown using anti-HA beads from 293T WT cells expressing empty 3×HA vector (empty) or 3×HA-PIP5K1A (PIP5K1A). The western blot shows the input (first two lanes) and the immunoprecipitated endogenous PIP4K2A with PIP5K1A (second two lanes). FIG. 3G shows proteins strongly pulled down detected by western blot to detect enrichment of PIP5K1A and PIP5K1C. The 293T lysates were incubated with beads pre-incubated with either GST or GST-PIP4K2C, used as bait. FIG. 3H illustrates the flow-through from the pulldown experiment described in FIG. 3G to show the extent to which lysate is depleted of PIP5K1A and PIP5K1C by the bait. FIG. 3I illustrates the sequence homology of the N-terminal regions in PIP4K. Mutated residues of PIP4K2C aa69-75 are indicated in red with underlining. The PIP4K2A peptide sequence. INELSHVQIPVMLMPDDFKAY SKIKV, has SEQ ID NO:1. The PIP4K2B peptide sequence, INELSNVPVPVMLMPDDFKAYSKIKV, has SEQ ID NO:2. The PIP4K2C peptide sequence, INELSQVPPPVMLLPDDF KASSKIKV, has SEQ ID NO:3. The PIP4K2CVD peptide sequence, INELSQVPPPETFLPNNFKASSKIKV, has SEQ ID NO:4. FIG. 3J illustrates bead pulldowns were analyzed by western blot for presence of PIP5K1A, which is able to bind WT PIP4K2C but not PIP4K2C^{VD} from 293T lysates that were incubated with beads conjugated to GST-PIP4K2C or GST-PIP4K2C^{VD} and used as bait. FIG. 3K graphically illustrates the results of an in vitro kinase assay that quantitatively measured conversion of PI(4)P to radiolabeled PI(4,5)P₂ by PIP5K1A, which is inhibited by PIP4K2C but not PIP4K2C^{VD}. FIG. 3L graphically illustrates the quantities of PI(4)P in HEK293T TKO cell lines rescued with PIP4K2C and PIP4K2C^{VD} as measured by HPLC. FIG. 3M graphically illustrates the quantities of PI(4,5)P₂ in HEK293T TKO cell lines rescued with PIP4K2C and PIP4K2C^{VD} as measured by HPLC. For FIGS. 3L-3M the significance calculated using ANOVA with Holm-Sidak multiple comparisons to control cell line. **p<0.01, ****p<0.0001, n.s. not significant. Data are represented as mean±SEM, n=3. FIG. 3N-3Q illustrate the mechanisms by which PIP4K may alter PIP5K activity. FIG. 3N graphically illustrates that expression of PIP4K2A^{KD} causes dose-dependent changes in phosphatidylinositol lipids, showing validation of low and high expression levels of 3×HA-PIP4K2A^{KD} in 293T WT or TKO cells. FIG. 3O graphically illustrates that expression of PIP4K2A^{KD} causes dose-dependent changes in phosphatidylinositol lipids showing measurement of PI(4,5)P₂ with low or high expression of PIP4K2A^{K}. FIG. 3P graphically illustrates that increased PIP5K activity cannot be explained by cellular levels of phosphatidic acid. FIG. 3Q graphically illustrates phosphatidic acid levels in TKD1 and TKD2 cells.

FIG. 4A-4K illustrate the structural role of PIP4K in regulating PIP5K and that the PI3K pathway is distinct from its catalytic role in autophagy. FIG. 4A graphically illustrates insulin stimulation of PI(3,4,5)P₃ synthesis by PI3K in 293T rescue cell lines, as quantified by HPLC. Cells were serum starved for 20 hours, then stimulated with 50 ng/mL insulin for 5 minutes. FIG. 4B shows western blots illustrating insulin stimulation of Akt activation in Hela rescue cell line panel. For FIGS. 4A-4B, cells were serum starved for 12 hours, then stimulated with 250 ng/mL insulin for 10 minutes. Western blot band intensities were measured by Licor and are indicated by the numbers under the relevant band. FIG. 4C graphically illustrates the insulin-stimulated increase in pAkt-473 in different Hela rescue cell lines, normalized to total Akt, n=3. FIG. 4D illustrates dependence on PIP4K catalytic activity to rescue increased lysosomal accumulation in images of 293T rescue cell lines where the immunofluorescence is of lysosomal marker Lamp1. FIG. 4E graphically illustrates the number of lysosomal puncta that express the marker Lamp1 in 293T rescue cell lines. FIG. 4F graphically illustrates gene expression levels of genes in genes targeted by TFEB as quantified by qRT-PCR in 293T rescue cell line panel. Significance calculated using ANOVA with Holm-Sidak multiple comparisons to control cell line. *p<0.05, **p<0.01, ***p<0.001, ****p<0.0001, n.s. not significant. FIG. 4G-4K illustrate testing and characterizing candidate domains of PIP4K in inhibiting PIP5K.

FIG. 4G graphically illustrates conversion of PI(4)P to radiolabeled PI(4,5)P₂ by PIP5K1A as quantified by in vitro kinase assays. The reaction was performed in the absence or presence of purified PIP4K2C or truncated PIP4K2C with a frame shift mutation at amino acid position132 (PIP4K2C*fs132). FIG. 4H graphically illustrates conversion of PI(4)P to radiolabeled PI(4,5)P₂ by PIP5K1A. The reaction was performed in the absence or presence of purified PIP4K2C and point mutants PIP4K2C. Significance calculated using ANOVA with Holm-Sidak multiple comparisons to control cell line, *p<0.05. FIG. 4I illustrates validation of properties of PIP4K2CVD. Coomassie confirming purity, size, and abundance of bacterial purified GST-PIP4K2C and GST-PIP4K2C^{VD}. FIG. 4J graphically illustrates conversion of PI(5)P to radiolabeled PI(4,5)P₂, confirming enzymatic activity of GST-PIP4K2CVD, as quantified by in vitro kinase assays. FIG. 4K illustrates validation of near-endogenous level expression of 3×-tagged PIP4K2C and PIP4K2C^{VD} in 293T TKO cells.

### DETAILED DESCRIPTION

As illustrated herein, PIP4Ks have a non-catalytic function that involves scaffolding with other cellular structures or proteins. The scaffolding function explains here-to-fore confusing aspects of PIP4K protein functions and provides more successful treatment methods and compositions for a variety of diseases and conditions. For example, targeted degradation or depletion of the PIP4K proteins (PIP4K2A PIP4K2B and PIP4K2C) can modulate cell growth, metabolism, and responses to external stimulation and growth signals. For example, by degrading PIP4Ks, PI3K signaling can be increased, which increases insulin sensitivity, reduces circulating glucose levels, and reduces obesity. PIP4K proteins also have a role in immune cell activation.

Degrading or depleting PIP4K proteins can alter immune responses that change the course of autoimmune disease and improve responses to infection or cancer. In some cases, a degraded or deleted form of PIP4K can be administered to a subject to alter immune responses and treat a variety of diseases and conditions. For example, the compositions and methods described herein can be used for treatment of diabetes, metabolic syndrome, insulin resistance, obesity, cancer, autoimmune disease, infection, or a combination thereof.

### PIP4K2A, PIP4K2B and PIP4K2C Enzymes

Phosphatidylinositol-4,5-bisphosphate (PI(4,5)P₂) plays numerous roles in cellular regulation. It mediates actin remodeling at the plasma membrane, modulates vesicle trafficking, and is the substrate that hormone-stimulated phospholipases type C (PLC) utilizes to generate the second messengers diacylglycerol and inositol-1,4,5-trisphosphate (Balla, 2013; Sun et al., 2013). PI(4,5)P₂ is also the substrate that Class 1 phosphoinositide 3-kinases use (Saito et al., 2003) to generate the second messenger phosphatidylinositol-3,4,5-trisphosphate (PI(3,4,5)P₃) in response to insulin and other growth factors (Fruman et al., 2017).

Yeast have a single enzyme for generating PI(4,5)P₂ encoded by MSS4 (Homma et al., 1998), whereas mammals have six genes encoding enzymes that generate PI(4,5)P₂. PIP5K1A, PIP5K1B and PIP5K1C produce PI(4,5)P₂ from phosphatidylinositol-4-phosphate (PI(4)P), while PIP4K2A, PIP4K2B and PIP4K2C generate PI(4,5)P₂ from phosphatidylinositol-5-phosphate (PI(5)P) (Rameh et al., 1997; van den Bout and Divecha, 2009). All multicellular organisms have genes from both families.

An example of a sequence for a human phosphatidylinositol 5-phosphate 4-kinase type-2 alpha (isoform 1) encoded by a human PIP4K2A gene is shown below (SEQ ID NO:6, NCBI accession no. NP_005019.2):

| | |
|---|---|
| 1 | MATPGNLGSS VLASKTKTKK KHFVAQKVKL FRASDPLLSV |
| 41 | LMWGVNHSIN ELSHVQIPVM LMPDDFKAYS KIKVDNHLFN |
| 81 | KENMPSHFKF KEYCPMVFRN LRERFGIDDQ DFQNSLTRSA |
| 121 | PLPNDSQARS GARFHTSYDK RYIIKTITSE DVAEMHNILK |
| 161 | KYHQYIVECH GITLLPQFLG MYRLNVDGVE IYVIVTRNVF |
| 201 | SHRLSVYRKY DLKGSTVARE ASDKEKAKEL PTLKDNDFIN |
| 241 | EGQKIYIDDN NKKVFLEKLK KDVEFLAQLK LMDYSLLVGI |
| 281 | HDVERAEQEE VECEENDGEE EGESDGTHPV GTPPDSPGNT |
| 321 | LNSSPPLAPG EFDPNIDVYG IKCHENSPRK EVYFMAIIDI |
| 361 | LTHYDAKKKA AHAAKTVKHG AGAEISTVNP EQYSKRFLDF |
| 401 | IGHILT |

The PIP4K N-terminal motif VMLXPDD (SEQ ID NO:5, where X is any amino acid) is highlighted in SEQ ID NO:6 in bold and with underlining. In some cases, the motif VMLXPDD (SEQ ID NO:5) can be deleted or degraded to generate a useful PIP4K polypeptide. In other cases, PIP4K peptides with the VMLXPDD (SEQ ID NO:5) motif but without other portions of the PIP4K polypeptide can be useful.

A cDNA encoding the PIP4K2A protein with (SEQ ID NO:6) can have the following nucleic acid sequence (SEQ ID NO:7, NM_005028.5).

| | |
|---|---|
| 1 | AGCCGCGGGC TGAGCGCGGA TCCGCGGCGG GCGCAGGATA |
| 41 | CGGGCCGGGG CGCGAGCCGA GCGCAGTCTG CCGGGCCGAG |
| 81 | CGGGCGGAGC GAGCCGAGTG GGGCTGAGCG CGCCGGCGGC |
| 121 | GGCGGGCGGA GCGGAGCGCG GCGCGCCGGG GCCGCCGCCG |
| 161 | GGGGGATGCG GCTGCCTCCC CGGGCCGGGG TGTAGAGAGG |
| 201 | GCGGGTCCCC GGCCTCGGGA GCACGGCGGT GGAGGGGACA |
| 241 | TAGGAGGCGG CCATGGCGAC CCCCGGCAAC CTAGGGTCCT |
| 281 | CTGTCCTGGC GAGCAAGACC AAGACCAAGA AGAAGCACTT |
| 321 | CGTAGCGCAG AAAGTGAAGC TGTTTCGGGC CAGCGACCCG |
| 361 | CTGCTCAGCG TCCTCATGTG GGGGGTAAAC CACTCGATCA |
| 401 | ATGAACTGAG CCATGTTCAA ATCCCTGTTA TGTTGATGCC |
| 441 | AGATGACTTC AAAGCCTATT CAAAAATAAA GGTGGACAAT |
| 481 | CACCTTTTTA ACAAAGAAAA CATGCCGAGC CATTTCAAGT |
| 521 | TTAAGGAATA CTGCCCGATG GTCTTCCGTA ACCTGCGGGA |
| 561 | GAGGTTTGGA ATTGATGATC AAGATTTCCA GAATTCCCTG |
| 601 | ACCAGGAGCG CACCCCTCCC CAACGACTCC CAGGCCCGCA |
| 641 | GTGGAGCTCG TTTTCACACT TCCTACGACA AAAGATACAT |
| 681 | CATCAAGACT ATTACCAGTG AAGACGTGGC CGAAATGCAC |
| 721 | AACATCCTGA AGAAATACCA CCAGTACATA GTGGAATGTC |
| 761 | ATGGGATCAC CCTTCTTCCC CAGTTCTTGG GCATGTACCG |
| 801 | GCTTAATGTT GATGGAGTTG AAATATATGT GATAGTTACA |
| 841 | AGAAATGTAT TCAGCCACCG TTTGTCTGTG TATAGGAAAT |
| 881 | ACGACTTAAA GGGCTCTACA GTGGCTAGAG AAGCTAGTGA |
| 921 | CAAAGAAAAG GCCAAAGAAC TGCCAACTCT GAAAGATAAT |
| 961 | GATTTCATTA ATGAGGGCCA AAAGATTTAT ATTGATGACA |
| 1001 | ACAACAAGAA GGTCTTCCTG GAAAAACTAA AAAAGGATGT |
| 1041 | TGAGTTTCTG GCCCAGCTGA AGCTCATGGA CTACAGTCTG |
| 1081 | CTGGTGGGAA TTCATGATGT GGAGAGAGCC GAACAGGAGG |
| 1121 | AAGTGGAGTG TGAGGAGAAC GATGGGGAGG AGGAGGGCGA |
| 1161 | GAGCGATGGC ACCCACCCGG TGGGAACCCC CCCAGATAGC |
| 1201 | CCCGGGAATA CACTGAACAG CTCACCACCC CTGGCTCCCG |
| 1241 | GGGAGTTCGA TCCGAACATC GACGTCTATG GAATTAAGTG |
| 1281 | CCATGAAAAC TCGCCTAGGA AGGAGGTGTA CTTCATGGCA |
| 1321 | ATTATTGACA TCCTTACTCA TTATGATGCA AAAAAGAAAG |
| 1361 | CTGCCCATGC TGCAAAAACT GTTAAACATG GCGCTGGCGC |
| 1401 | GGAGATCTCC ACCGTGAACC CAGAACAGTA TTCAAAGCGC |
| 1441 | TTTTTGGACT TTATTGGCCA CATCTTGACG TAACCTCCTG |
| 1481 | CGCAGCCTCG GACAGACATG AACATTGGAT GGACACAGGT |
| 1521 | GGCTTCGGTG TAGGAAAAAT GAAAACCAAA CTCAGTGAAG |
| 1561 | TACTCATCTT GCAGGAAGCA AACCTCCTTG TTTACATCTT |
| 1601 | CAGGCCAAGA TGACTGATTT GGGGGCTACT CGCTTTACAG |
| 1641 | CTACCTGATT TTCCCAGCAT CGTTCTAGCT ATTTCTGACT |
| 1681 | TTGTGTATAT GTGTGTGTGT GTGTGTTGGG GGGGGGTGAG |
| 1721 | TGTGTGCGCG CGTGTGCATT TTAAAAGTCA TAAATTAATT |
| 1761 | AAAACAGATC CACTTCGGTC AGTATGTGTC CCAACAAAGA |
| 1801 | CCCTTTGATT CCAGCTATGG CCGAATGAAT GAGTGAGTGA |
| 1841 | GTGAGTGAGT GAATGAACAC ACGTGTGGGG GAGGGGAGAA |
| 1881 | GGAAGTGCAT GATGTCAGGC ACCGTGTTGG CATCACACAA |
| 1921 | CAAACTGTGG ATCAGTTTTT TTTTTTTTTT TTTTTTTTTG |
| 1961 | GAGTTGAAAG ATGTGAGACA GTATTCAGAA TAATGAAGAT |
| 2001 | AATAATGATG ATTATTATAA TAATGATGAT GATTCCAAGG |
| 2041 | AAAAAACCTA CAGCGAATGT TCCATTTCTA CCCCGCACGC |
| 2081 | AGACACTCTC CCTAACACTG ATAACCTGAG CCCCCAGCAC |
| 2121 | TGGACGGAAG AATGCTGGCG TCTCCGTGTG TACTGGTTCA |
| 2161 | GGGTTCTGGC CCCAGCCTTG TCAGGACCCC CTGGTGTCCA |
| 2201 | GAGCCCCCAC CCCTCCCGCA ACAAGCAGCT GATGCCCCAG |
| 2241 | TGATTCTCTA TACATTTTTC ACCTCGGCCA ATATGTCCAG |
| 2281 | GAAAACTGCT TACTTCTCTT TTCTTGCCTG GAGCCTTCAT |
| 2321 | TGTTCACCCT TACGTTGCAA TATAGGAATT AATGCTACAA |
| 2361 | AATAAAAGTA AAGCTTACCT GAAAAGTGCA TAGTTTGGGG |
| 2401 | CAATGGTATC TACATCTCCC ACTGTGGGAA AACCAGCAAA |
| 2441 | GCATCAAAAC TCTCAATTCT CCTGTTACCA AATGCAGATC |
| 2481 | TGAATTATAA GATGTTTATG TTTGACCATT GTTTCAACAA |
| 2521 | TGGGATTTTG TTACGAATTA TCCCTTTAAC TGAAACCCTC |
| 2561 | AGTTTTACTG TTTACATTAT TAGGAAAACA GGGATATCTT |
| 2601 | TTGAATCTAA AAATTTGATG TACAGCATGT GATTTTTGAA |
| 2641 | GTTTACATGT AAAGTCACAG TATAGGTGAA ATAACGTTTG |
| 2681 | TCATATTTTG AGACGTATCC TGCAGCCATG TTTTTACGTG |
| 2721 | AGTGTTTTAG TCAAAGTACA TGGTAGACAG TCTTTCACAA |
| 2761 | TAAAAGGAAA AGGATTTTTT TTTCCTCCAA ATGTACATTT |
| 2801 | ATCAACCTAA TGATTGATTT TTTTAAAAAG AGATTTCGCC |
| 2841 | CCAGTCTGGT TTATGAAAGT TCATTGCCCT AAACTGTGCT |
| 2881 | GATTGTTTTT AATCAAGTTA TAAATTTCCA ACCTAGATCA |
| 2921 | TGTATCTACC AACTCTCCTG CATTTTCCAA AAGGCATTGA |
| 2961 | GCTTAAATAT TAGTCTTGCT TAGAGTAGGT TATCCACTTA |
| 3001 | CATGCTGCGC TAAAGCCATG CCTTTGAAAC TCCTTGTTTA |
| 3041 | AAACATGATA TGATTTTTGT GGGCAGTTTC AGAAAAGAAA |
| 3081 | ACAAACAAAC AAAAATCGAC CCTTTAATTA TTACTTGCAA |
| 3121 | CTCAACAGAT CTCCCTGCCG TACTGCCTTT TCCAGGAACT |
| 3161 | TTACTTCAGG GCTGTCCAGA TTGCAGCTGT GCCCCGTGTA |
| 3201 | TGTGGATCTA GTTCACAGAG TCTTTGGAAG CCAGCAGTCG |
| 3241 | TGCCCTCCGT ATACTGTCCA CTCATTTTAT GTAGATTTGG |
| 3281 | TATCCTCAGC AGCCAGTGTT AACACCACTG TCACGTAGTG |
| 3321 | TACAGATTCA TCTTTTATGT ATTTAAAGTA ATCCATACTA |
| 3361 | TGATTTGGTT TTTCCCTGCA CCATTAATTC TGGCATCAGA |
| 3401 | TCAGTTTTTG TGTTGTGAAG TTCTACTGTG GTTTGACCCA |
| 3441 | AGACCACAAC CATGAGACCC TGAAGTAAAG ATAAGGTACA |
| 3481 | CATACATTAT TTGAGTAACT GTTTCCTTGG GGGCCAATCT |
| 3521 | GTGTATGCTT TTAGAAGTTT ACAGAATGCT TTTATTTTTG |
| 3561 | TCTATAACAA ACAGTCTGTC ATTTATTTCT GTTGATAAAC |
| 3601 | CATTTGGACA GAGTGAGGAC GTTTGCCCTG TTATCTCCTA |
| 3641 | GTGCTAACAA TACACTCCAG TCATGAGCCG GGCTTTACAA |
| 3681 | ATAAAGCACT TTTGATGACT CACAAGATGA ATCCTTTTTT |
| 3721 | CCTCTGTCCC AATTGTGTGT CTCTGTTCCA AACACATTTT |
| 3761 | AAATACTCGG TCCTGACAGT GTCTTTAGCT AATCCTTGAA |
| 3801 | GAAATGAAAG TGGAATTGAA |

Other isoforms of the PIP4K2A protein exist and include NCBI accession nos. NP_001316991.1 (GI: 1052292374); XP_006717513.1 (GI: 578818419); XP_016871820.1 (GI: 1034568484); XP_016871819.1 (GI: 1034568482); and XP_016871821.1 (GI: 1034568486). Any of these PIP4K2A proteins can be modified or targeted to reduce expression and/or reduce function thereof.

An example of a sequence for a human phosphatidylinositol 5-phosphate 4-kinase type-2 beta encoded by a human PIP4K2B gene is shown below (SEQ ID NO:8, NCBI accession no. NP_003550.1).

| | |
|---|---|
| 1 | MSSNCTSTTA VAVAPLSASK TKTKKKHFVC QKVKLFRASE |
| 41 | PILSVLMWGV NHTINELSNV PVPVMLMPDD FKAYSKIKVD |
| 81 | NHLFNKENLP SRFKFKEYCP MVFRNLRERF GIDDQDYQNS |
| 121 | VTRSAPINSD SQGRCGTRFL TTYDRRFVIK TVSSEDVAEM |
| 161 | HNILKKYHQF IVECHGNTLL PQFLGMYRLT VDGVETYMVV |
| 201 | TRNVFSHRLT VHRKYDLKGS TVAREASDKE KAKDLPTFKD |
| 241 | NDFLNEGQKL HVGEESKKNF LEKLKRDVEF LAQLKIMDYS |
| 281 | LLVGIHDVDR AEQEEMEVEE RAEDEECEND GVGGNLLCSY |
| 321 | GTPPDSPGNL LSFPRFFGPG EFDPSVDVYA MKSHESSPKK |
| 361 | EVYFMAIIDI LTPYDTKKKA AHAAKTVKHG AGAEISTVNP |
| 401 | EQYSKRFNEF MSNILT |

The PIP4K N-terminal motif VMLXPDD (SEQ ID NO:5, where X is any amino acid) is highlighted in SEQ ID NO:8 in bold and with underlining. As indicated above, in some cases, the motif VMLXPDD (SEQ ID NO:5) can be deleted or degraded to generate a useful PIP4K polypeptide. In other cases, PIP4K peptides with the VMLXPDD (SEQ ID NO:5) motif but without other portions of the PIP4K polypeptide can be useful. The PIP4K2B protein is expressed in muscle at higher levels than the PIP4K2A or PIP4K2C. Hence, degradation or depletion of the PIP4K2B may be more useful for treatment of insulin resistance and obesity than targeting PIP4K2A or PIP4K2C for degradation.

A cDNA encoding the PIP4K2B protein with (SEQ ID NO:8) can have the following nucleic acid sequence (SEQ ID NO:9, NM_003559.4).

| | |
|---|---|
| 1 | TTGCGGGAAA GAGCCAAACC CTGGCGTTGG GGGGCCCGGG |
| 41 | CGGGGAGCCC CTCCCGCGGT CCACAGCGAC GCCTGCCCAG |
| 81 | CCCTCCTCCC CTTCCGGCTC CGGCACGGGG CCCCGAGGCG |
| 121 | TTCGGAGGCC AGGCGGGTTT CTGTCAGGCC CGGGGAGGAG |
| 161 | GGGCGGGCGG GGCGGCCGCT GCCTCCCCGG GACGGGCCGT |
| 201 | ACCACGCGGA CGGGGAGGAC GGGGCCAGGG GACTGCAGGG |
| 241 | CGGCTGCACC GCCCGGGGGC GGGGTGCGGA GCGGGCCGGC |
| 281 | GGGCTCCCCG GGGCGGGGCG GGAGGGCGGG GCGTGGGGCG |
| 321 | GACGGAACCA CCGGGGCGGG GTGGGAGGTA ACGGGACGGG |
| 361 | CGCGACCATG GCGCGGTGAG GGAGCGGGGG TGGGGATCGG |
| 401 | TCCGGGGGAG GCCTGAGGCC GCTGGCTTGT GCGCTGTCTC |
| 441 | CGCCGCCCCC CTCTTTCGCC GCCGCCGCCG CCGCCCCGGG |
| 481 | CATGTCGTCC AACTGCACCA GCACCACGGC GGTGGCGGTG |
| 521 | GCGCCGCTCA GCGCCAGCAA GACCAAGACC AAGAAGAAGC |
| 561 | ATTTCGTGTG CCAGAAAGTG AAGCTATTCC GGGCCAGCGA |
| 601 | GCCGATCCTC AGCGTCCTGA TGTGGGGGGT GAACCACACG |
| 641 | ATCAATGAGC TGAGCAATGT TCCTGTTCCT GTCATGCTAA |
| 681 | TGCCAGATGA CTTCAAAGCC TACAGCAAGA TCAAGGTGGA |
| 721 | CAATCATCTC TTCAATAAGG AGAACCTGCC CAGCCGCTTT |
| 761 | AAGTTTAAGG AGTATTGCCC CATGGTGTTC CGAAACCTTC |
| 801 | GGGAGAGGTT TGGAATTGAT GATCAGGATT ACCAGAATTC |
| 841 | AGTGACGCGC AGCGCCCCCA TCAACAGTGA CAGCCAGGGT |
| 881 | CGGTGTGGCA CGCGTTTCCT CACCACCTAC GACCGGCGCT |
| 921 | TTGTCATCAA GACTGTGTCC AGCGAGGACG TGGCGGAGAT |
| 961 | GCACAACATC TTAAAGAAAT ACCACCAGTT TATAGTGGAG |
| 1001 | TGTCATGGCA ACACGCTTTT GCCACAGTTC CTGGGCATGT |
| 1041 | ACCGCCTGAC CGTGGATGGT GTGGAAACCT ACATGGTGGT |
| 1081 | TACCAGGAAC GTGTTCAGCC ATCGGCTCAC TGTGCATCGC |
| 1121 | AAGTATGACC TCAAGGGTTC TACGGTTGCC AGAGAAGCGA |
| 1161 | GCGACAAGGA GAAGGCCAAG GACTTGCCAA CATTCAAAGA |
| 1201 | CAATGACTTC CTCAATGAAG GGCAGAAGCT GCATGTGGGA |
| 1241 | GAGGAGAGTA AAAAGAACTT CCTGGAGAAA CTGAAGCGGG |
| 1281 | ACGTTGAGTT CTTGGCACAG CTGAAGATCA TGGACTACAG |
| 1321 | CCTGCTGGTG GGCATCCACG ACGTGGACCG GGCAGAGCAG |
| 1361 | GAGGAGATGG AGGTGGAGGA GCGGGCAGAG GACGAGGAGT |
| 1401 | GTGAGAATGA TGGGGTGGGT GGCAACCTAC TCTGCTCCTA |
| 1441 | TGGCACACCT CCGGACAGCC CTGGCAACCT CCTCAGCTTT |
| 1481 | CCTCGGTTCT TTGGTCCTGG GGAATTCGAC CCCTCTGTTG |
| 1521 | ACGTCTATGC CATGAAAAGC CATGAAAGTT CCCCCAAGAA |
| 1561 | GGAGGTGTAT TTCATGGCCA TCATTGATAT CCTCACGCCA |
| 1601 | TACGATACAA AGAAGAAAGC TGCACATGCT GCCAAAACGG |
| 1641 | TGAAACAEGG GGCAGGGGCC GAGATCTCGA CTGTGAACCC |
| 1681 | TGAGCAGTAC TCCAAACGCT TCAACGAGTT TATGTCCAAC |
| 1721 | ATCCTGACGT AGTTCTCTTC TACCTTCAGC CAGAGCCAGA |
| 1761 | GAGCTGGATA TGGGGTCGGG GATCGGGAGT TAGGGAGAAG |
| 1801 | GGTGTATTTG GGCTAGATGG GAGGGTGGGA GCAGAGTCGG |
| 1841 | GTTTGGGAGG GCTTTAGCAA TGAGACTGCA GCCTGTGACA |
| 1881 | CCGAAAGAGA CTTTAGCTGA AGAGGAGGGG GATGTGCTGT |
| 1921 | GTGTGCACCT GCTCACAGGA TGTAACCCCA CCTTCTGCTT |
| 1961 | ACCCTTGATT TTTTCTCCCC ATTTGACACC CAGGTTAAAA |
| 2001 | AGGGGTTCCC TTTTTGGTAC CTTGTAACCT TTTAAGATAC |
| 2041 | CTTGGGGCTA GAGATGACTT CGTGGGTTTA TTTGGGTTTT |
| 2081 | GTTTCTGAAA TTTCATTGCT CCAGGTTTGC TATTTATAAT |
| 2121 | CATATTTCAT CAGCCTACCC ACCCTCCCCA TCTTTGCTGA |
| 2161 | GCTCTCAGTT CCCTTCAATT AAAGAGATAC CCGGTAGACC |
| 2201 | CAGCACAAGG GTCCTTCCAG AACCAAGTGC TATGGATGCC |
| 2241 | AGATTGGAGA GGTCAGACAC CTCGCCCTGC TGCATTTGCT |
| 2281 | CTTGTCTGGA TTAACTTTGT AATTTATGGA GTATTGTGCA |
| 2321 | CAACTTCCTC CACCTTTCCC TTGGATTCAA GTGAAAACTG |
| 2361 | TTGCATTATT CCTCCATCCT GTCTGGAATA CACCAGGTCA |
| 2401 | ACACCAGAGA TCTCAGATCA GAATCAGAGA TCTCAGAGGG |
| 2441 | GAATAAGTTC ATCCTCATGG GATGGTGAGG GGCAGGAAAG |
| 2481 | CGGCTGGGCT CTTGGACACC TGGTTCTCAG AGAACCCTGT |
| 2521 | GATGATCACC CAAGCCCCAG GCTGTCTTAG CCCCTGGAGT |
| 2561 | TCAGAAGTCC TCTCTGTAAA GCCTGCCTCC CACTAGGTCA |
| 2601 | AGAGGAACTA GAGTACCTTT GGATTTATCA GGACCCTCAT |
| 2641 | GTTTAAATGG TTATTTCCCT TTGGGAAAAC TTCAGAAACT |
| 2681 | GATGTATCAA ATGAGGCCCT GTGCCCTCGA TCTATTTCCT |
| 2721 | TCTTCCTTCT GACCTCCTCC CAGGCACTCT TACTTCTAGC |
| 2761 | CGAACTCTTA GCTCTGGGCA GATCTCCAAG CGCCTGGAGT |
| 2801 | GCTTTTTAGC AGAGACACCT CGTTAAGCTC CGGGATGACC |
| 2841 | TTGTAGGAGA TCTGTCTCCC TGTGCCTGGA GAGTTACAGC |
| 2881 | CAGCAAGGTG CCCCCATCTT AGAGTGTGGT GTCCAAACGT |
| 2921 | GAGGTGGCTT CCTAGTTACA TGAGGATGTG ATCCAGGAAA |
| 2961 | TCCAGTTTGG AGGCTTGATG TGGGTTTTGA CCTGGCCTCA |
| 3001 | GCCTTGGGGC TGTTTTTCCT TGTTGCCCCG CTCTAGACTT |
| 3041 | TTAGCAGATC TGCAGCCCAC AGGCTTTTTT GGAAGGAGTG |
| 3081 | GCTTCCTGCA GGTGTTCCAC CTGCCTTCGG AGCCTGCCAC |
| 3121 | CCAGGCCCTC AGAACTGAGC CACAGGCTGC TCTGGCCAGG |
| 3161 | AGAGAAACAG CTCTGTTGTT CTGCATTGGG GGAGGTACAT |
| 3201 | TCCTGCATCT TCTCACCCCC TCAACCAGGA ACTGGGGATT |
| 3241 | TGGGATGAGA TATGGTCAGA CTTGTAGATA ACCCCAAAGA |
| 3281 | TGTGAAGATC GCTTGTGAAA CCATTTTGAA TGAATAGATT |
| 3321 | GGTTTCCTGT GGCTCCCTCC AAACCTGGCC AAGCCCAGCT |
| 3361 | TCCGAAGCAG GAACCAGCAC TGTCTCTGTG CCTGACTCAC |
| 3401 | AGCATATAGG TCAGGAAAGA ATGGAGACGG CATTCTTGGA |
| 3441 | CTTCACTGGG GCTGCTGGAT TGGATGGGAA ACCTTCTGGA |
| 3481 | AGAGGCAGAT GGGGGTCAAA CCACTGCCTT GGCCCCAGGA |
| 3521 | AGGGGCCATA GGTAGGTCTG AACAACTGCC GCAAGACCAC |
| 3561 | TACATGACTT AGGGAACTTG AAACCAACTG GCTCATGGAG |
| 3601 | AAAACAAATT TGACTTGGGA AAGGGATTAT GTAGGAATAA |
| 3641 | TGTTTGGACT TGATTTCCCC ACGTCATAAT GAAGAATGGA |
| 3681 | AGTTTGGATC TGCTCCTCGT CAGGCGCAGC ATCTCTGAAG |
| 3721 | CTTGGAAAGC TGTCTTCCAG CAGCCTCCGT GGCCTCGGGT |
| 3761 | TCCTACCGGC TTCTCTGCAT TTGGTCTGCT GATCATGTTG |
| 3801 | CCATAATGTG TATGGAAAGT GTAACACATT CTTACTGGTT |
| 3841 | AAAGACGACT ACCAGGTATC TAACTTGTTT AACATTGAGT |
| 3881 | TTGTGTGTGT GTGTGTATGT TTGTGTGTTT TGTATATTGT |
| 3921 | TTACATTTTG AGAGGTAGCA TTCTGTTTCA AATGCTTTTT |
| 3961 | GTTTTTCTGA CAGTATTGTT GACTGGGTCA TAACATTTTG |
| 4001 | AGCTGTGGTT TGGTGGATTT TCAATTTTTT TTTTTAAAGG |
| 4041 | TCATTCGCTG TGCTATCTTC AAAACCTTGA GTTTGGCCCC |
| 4081 | CAATTTTTGG CATTCAAATG TTTAAAAGCT ATTTATCTTG |
| 4121 | GTTTATACAA GTTTCCTTTC TCTTCTTTTT GTCATGGTAT |
| 4161 | TCTATTTGGT CTGCAGTTTG AATGTAGAGA AAGTGGACTG |
| 4201 | ATCCCCCAAG CGTTGTCTGC CCCCACTCTT TCCTCCTTGG |
| 4241 | GTCCCGCCAT TCTTTTACTG GGCAGTCGAG GGCATTGGAG |
| 4281 | GGGAAGTGAC TGCCCTCAGC CTCACTCCCT GGGGCCATGA |
| 4321 | AGAAAAGCTA AACAGTCTCA TGGCATCTCA GAATAATGTT |
| 4361 | GGGTCTCCCA AGAAGAAAGG TGTAAGAATA ACGACATGGC |
| 4401 | TGATTAGGCG AGGCCAGGAT AGGGCTAAGG CCAGGATTCC |
| 4441 | TGGCTGGCAT CCAGTCACCC CTTCTCCCAT CCTTCCCCCT |
| 4481 | CTTCTTCCAC AAGTCCGCAG CCGAGACACT GTAGTCTCCC |
| 4521 | AGCCACAGTG ATGAGTGCCC TGGAGACTCC ACTGACCTCT |
| 4561 | AGATGAAGGC CCCTGGCCCT GGTTCCTGTT AATTAACCTC |
| 4601 | TGGGTCTTTG AGTCCCCCAG CACAAACTTC TTTCCTGTAC |
| 4641 | CCTGCGGCTT GGGGTCACAG GGCATGCCGG GAAGCCACAG |
| 4681 | CTGAGGGGCG CAGACTGAAG CAGTGCTCCA CCTCTCCTTC |
| 4721 | TTTAGCTCAG GGGTTGCTGG TCTGTGGCAG GCGCCACGAG |
| 4761 | TGGCCCCTGT GGCTGTTCTC AGTGGCAGTC TCTTAAGTTC |
| 4801 | CCACCACAGG CAGCTCTTTA TCCCCTCTCC CTACTTGACT |
| 4841 | CTTTCTCTTG CCTGTGCTTT TGGCCTCAAA CAGGCCTGCT |
| 4881 | GGTAGCGCTC AGGGCGTGAG GCTACACTCC TGCCCTGCCT |
| 4921 | TTCCTGTCTT CATGGTCTGC CAGGGCATAC CTTGGGGAGG |
| 4961 | TGGACCAAAG ACCCAGGACT TTTTGCAGTA GCCAGTCCTA |
| 5001 | CCCCCCAGTT GTCTTTTTAC CAATTCAGGG TGGGAGAGAA |
| 5041 | AACTGCAGCA CCCTAGCATG TGAGTTACTC AGGTGTTGGG |
| 5081 | GGCTAGAAGG GACAGTGCGT TTAAACAACA CTCAGAGCTC |
| 5121 | TGGCCTTAAA CCTGTGGCCC CCCAAGTCTA GGAGCCTCAT |
| 5161 | CTCTTCCTGG CAGTCATGCG GGCAGGAGGT CCTGAAAGGG |
| 5201 | AAAACCCATT CAGACAACTG TTCCCCAATC TACCAGCCAT |
| 5241 | CTGCAGGGGT CAGTGACCGT GGCCCTCTCC CTCCTCTAGA |
| 5281 | ATGTGCCACT TATGAAGAGT GCCCCATGGG GAAAAGGAGA |
| 5321 | CTCAGCTGTC CCTTGGCAGC TTGTGCCAGT ATCCCAGGGC |
| 5361 | AGAAGTTTCC ACAGGAGCCT CTTGCCCTTG CGCAGAGCCA |
| 5401 | CTGTGAGAGG CGGTGGGAGC CAACACCCTT GGGGGAGGGG |
| 5441 | GCAGTACTGC TCGGCACATC CCAGCATCAG GTCAGATCAT |
| 5481 | TGAAATTAAA AAATGTGAAT TAAGTTCATA TCCACCTTTT |
| 5521 | GGGGAAGCAG GACAAACCAC CACCCCACCA AGTGTGTGAC |
| 5561 | TTCTCCATAT CCCACTGCAG TTTCCATTTT TTAAATGGGA |
| 5601 | ATTTTCAATC CCCTGTGCTT GTCTAACGTC TGCTTTAAAA |
| 5641 | AGTTTGAGAC CCTGTTACTG TTTGAAAATG CATGCATGTT |
| 5681 | ACGATGAATC TCCAACCTGA GGAAAAAAAT AAAACTCAAA |
| 5721 | AAGCTTTGTG TA |

Other isoforms of the PIP4K2A protein exist and include NCBI accession nos. XP_011523628.1 (GI: 767996099); XP_011523629.1 (GI: 767996101); XP_011523632.1 (GI: 767996107); XP_016880686.1 (GI: 1034601614); and/or XP_016880688.1 (GI: 1034601618). Any of these PIP4K2B proteins can be modified or targeted to reduce expression and/or reduce function thereof.

An example of a sequence for a human phosphatidylinositol 5-phosphate 4-kinase type-2 gamma encoded by a human PIP4K2C gene is shown below (SEQ ID NO:10, NCBI accession no. NP_079055.3).

| | |
|---|---|
| 1 | MASSSVPPAT VSAATAGPGP GFGFASKTKK KHFVQQKVKV |
| 41 | FRAADPLVGV FLWGVAHSIN ELSQVPPPVM LLPDDFKASS |
| 81 | KIKVNNHLFH RENLPSHFKF KEYCPQVFRN LRDRFGIDDQ |
| 121 | DYLVSLTRNP PSESEGSDGR FLISYDRTLV IKEVSSEDIA |
| 161 | DMHSNLSNYH QYIVKCHGNT LLPQFLGMYR VSVDNEDSYM |
| 201 | LVMRNMFSHR LPVHRKYDLK GSLVSREASD KEKVKELPTL |
| 241 | KDMDFLNKNQ KVYIGEEEKK IFLEKLKRDV EFLVQLKIMD |
| 281 | YSLLLGIHDI IRGSEPEEEA PVREDESEVD GDCSLTGPPA |
| 321 | LVGSYGTSPF GIGGYIHSHR PLGPGEFESF IDVYAIRSAE |
| 361 | GAPQKEVYFM GLIDILTQYD AKKKAAHAAK TVKHGAGAEI |
| 401 | STVHPEQYAK RFLDFITNIF A |

The PIP4K N-terminal motif VMLXPDD (SEQ ID NO:5, where X is any amino acid) is highlighted in SEQ ID NO:10 in bold and with underlining. In some cases, the motif VMLXPDD (SEQ ID NO:5) can be deleted or degraded to generate a useful PIP4K polypeptide. In other cases, PIP4K peptides with the VMLXPDD (SEQ ID NO:5) motif but without other portions of the PIP4K polypeptide can be useful.

The PIP4K2C protein is expressed in immune cells at higher levels than the PIP4K2A or PIP4K2B. Hence, degradation or depletion of the PIP4K2C may be more useful for treatment of immune system conditions and cancer than targeting PIP4K2A or PIP4K2B for degradation.

A cDNA encoding the PIP4K2C protein with (SEQ ID NO: 10) can have the following nucleic acid sequence (SEQ ID NO: 11, NM_024779.5).

| | |
|---|---|
| 1 | GGTCACGTGA CAGCAGCGCA GGTGAGCGCC GCTTCCGGGG |
| 41 | TCGGGCGCCT GGATAGCTGC CGGCTCCGGC TTCCACTTGG |
| 81 | TCGGTTGCGC GGGAGACTAT GGCGTCCTCC TCGGTCCCAC |
| 121 | CAGCCACGGT ATCGGCGGCG ACAGCAGGCC CCGGCCCAGG |
| 161 | TTTCGGCTTC GCCTCCAAGA CCAAGAAGAA GCATTTCGTG |
| 201 | CAGCAGAAGG TGAAGGTGTT CCGGGCGGCC GACCCGCTGG |
| 241 | TGGGTGTGTT CCTGTGGGGC GTAGCCCACT CGATCAATGA |
| 281 | GCTCAGCCAG GTGCCTCCCC CGGTGATGCT GCTGCCAGAT |
| 321 | GACTTTAAGG CCAGCTCCAA GATCAAGGTC AACAATCACC |
| 361 | TTTTCCAGAG GGAAAATCTG CCCAGTCATT TCAAGTTCAA |
| 401 | GGAGTATTGT CCCCAGGTCT TCAGGAACCT CCGTGATCGA |
| 441 | TTTGGCATTG ATGACCAAGA TTACTTGGTG TCCCTTACCC |
| 481 | GAAACCCCCC CAGCGAAAGT GAAGGCAGTG ATGGTCGCTT |
| 521 | CCTTATCTCC TACGATCGGA CTCTGGTCAT CAAAGAAGTA |
| 561 | TCCAGTGAGG ACATTGCTGA CATGCATAGC AACCTCTCCA |
| 601 | ACTATCACCA GTACATTGTG AAGTGCCATG GCAACACGCT |
| 641 | TCTGCCCCAG TTCCTGGGGA TGTACCGAGT CAGTGTGGAG |
| 681 | AACGAAGACA GCTACATGCT TGTGATGCGC AATATGTTTA |
| 721 | GCCACCGTCT TCCTGTGCAC AGGAAGTATG ACCTCAAGGG |
| 761 | TTCCCTAGTG TCCCGGGAAG CCAGCGATAA GGAAAAGGTT |
| 801 | AAAGAATTGG CCACCCTTAA GGATATGGAC TTTCTCAACA |
| 841 | AGAACCAGAA AGTATATATT GGTGAAGAGG AGAAGAAAAT |
| 881 | ATTTCTGGAG AAGCTGAAGA GAGATGTGGA GTTTCTAGTG |
| 921 | CAGCTGAAGA TCATGGACTA CAGCCTTCTG CTAGGCATCC |
| 961 | ACGACATCAT TCGGGGCTCT GAACCAGAGG AGGAAGCGCC |
| 1001 | CGTGCGGGAG GATGAGTCAG AGGTGGATGG GGACTGCAGC |
| 1041 | CTGACTGGAG CTCCTGCTCT GGTGGGCTCC TATGGCACCT |
| 1081 | CCCCAGAGGG TATCGGAGGC TACATCCATT CCCATCGGCC |
| 1121 | CCTGGGCCCA GGAGAGTTTG AGTCCTTCAT TGATGTCTAT |
| 1161 | GCCATCCGGA GTGCTGAAGG AGCCCCCCAG AAGGAGGTCT |
| 1201 | ACTTCATGGG CCTCATTGAT ATCCTTACAC AGTATGATGC |
| 1241 | TAAGAAGAAA GCAGCTCATG CAGCCAAAAC TGTCAAGCAT |
| 1281 | GGGGCTGGGG CAGAGATCTC TACTGTCCAT CCGGAGCAGT |
| 1321 | ATGCTAAGCG ATTCCTGGAT TTTATTACCA ACATCTTTGC |
| 1361 | CTAAGAGACT GCCTGGTTCT CTCTGATGTT CAAGGTGGTG |
| 1401 | GGGTTCTGAG ACACTTGGGG GAATTGTGGG GATATTCTAG |
| 1441 | CCACCAGTTC TCTTCTTCCT TTGCTAAATT CAGGCTGCAG |
| 1481 | GCTCCTTCCA TCCAGATAAC TCCATCCTGT CGAGTAGGCT |
| 1521 | CTTTCTGACC CTCAGAAATA CATTGTCCTT TTTCCTCTTT |
| 1561 | GCCCATTTTT CTTCCCTCTC TTCCTCCCCA TGAGAAGTCT |
| 1601 | GCTTGTAGTA TTAGAATGTT ATTGTTGACT CTCTCCCAAG |
| 1641 | TGCCTTGATC TTTGTAATAT CTCCTGTTGT TTCTATGATA |
| 1681 | TAGGAGCTAG GGGAAGGGGG TTGTTTGCCT TCTTCAGGAC |
| 1721 | CTGACTGGAC AGATGGACCT GGCTCAAGCA ACTACTCTGG |
| 1761 | ATGCACTTTG CTGTGTGGGA TGAACTAAAA GTGTCTGAAT |
| 1801 | TTTGCTGATA ACTTTATAAA ACTCACTATG GCATGCTTCC |
| 1841 | CTCCTGGTGG GCCCTAGGAT GGATGACACT CAAGATACTA |
| 1881 | CAGATGTGGG TGCAGGCATG CACACACACG ATGGAATATG |
| 1921 | GCCATTCCTA CACAGGTGGG GTAGAGAGTG GGTCAGCAGC |
| 1961 | CTGGCACCTC ACAGAGGTGG GACCTAAGAG GACTCATGAT |
| 2001 | TATGCAGAGA ATTGGATTGG GTCTCTGTCA TAGATTGAGT |
| 2041 | AATCTCTTCC CTTACCTCAA TTCCATCTCC ACCCATCTCT |
| 2081 | ACATCTGGGC ACAGCAACCC AGAGATGGCC AAAAGCATTC |
| 2121 | AAGCCTGGGG GAAGATGTTT GACTATTGCT GCTCTTCACC |
| 2161 | AGAACCTCAC ACCTCTCCTG GGACTGGAAC CCTTCAGTGG |
| 2201 | GTGTGTGGCC AGTTTTGGAG GCTGGAATGA TGGGCCAGGG |
| 2241 | TGTAGGATTC ATTCTCCATG TAAAGTTTCC TTTCATCCTG |
| 2281 | CCTAGCCATC CCCAAGGTTT ATTTCCAGAA GAAAGGAATA |
| 2321 | TCTCTACTTG GATCAATTCT GGTCATTTCA AGAGGATGGA |
| 2361 | GGCCTCAAGT GTGGGAACTT CCCCTACTCC CTGGATGTGT |
| 2401 | GTACCTAGCA CACTTCCTTC TCCCACCCCT TTTTCCAGTT |
| 2441 | GGATTTGTTT TTCTGTTCTC TTCTGTCCTG TCTTATACTG |
| 2481 | CAACTGTGTC TCCTAGGGGA CAGATGGCCT TCTTTGTCAT |
| 2521 | CTTCACTCTC CACCCCCAGA GAGGAGTCAG AGCCATAACT |
| 2561 | CAATCACTCA GCCCCTCCAA AGATAGTTGA TGTGTGATAA |
| 2601 | TCTCATAATG TTGAGAACCC TGATGAGATA CATTGTCTTC |
| 2641 | CTCTCCCTAC AATGCCTCTG GGGCCAAGGC ACCCATTCTT |
| 2681 | CTTGCTATCC TCCATCCCCC TTGAGGCTTC CACTTTTTTT |
| 2721 | TTTTTTAGAC ATAAAGCTGG GCATCAGCAA CTGGCCTGTG |
| 2761 | GTGATGCAAA GCTGCTTTGC TCTGTATCTG GCTGGACTGA |
| 2801 | TCTGTCTCAC AAGAAGCCAT GAGGCCATAG GGAGAAGCTC |
| 2841 | CCTCTCCCCT TCATCTTCTG CTCCAAAGGT GGTAGCAAGA |
| 2881 | GGAGTACCCA GTTAGGGGTT GGAGCCCCCA TATAACATCT |
| 2921 | TCCTGTCAGA AGACTGATGG ATCTTTTTCA TTCCAACCAT |
| 2961 | CTCCCTTTCC CCCGATGAAT GCAATAAAAC TCTGTGACAC |
| 3001 | CAGCAACCAT TGCTCTTTAG AAATGGGTTT TCTGATCATA |
| 3041 | TGGCTGATGT GTTATGGGCA GTATGGATGT CTTCATTTGT |
| 3081 | TGCTTCTGTT TTTCATCTTT TTTGTTTTAT TAATAAAAAT |
| 3121 | TTATGTATTT GCTCCTGTTA CTATAATAAT ACAGGGAATA |
| 3161 | AATTATTCAA TCCAAA |

Other isoforms of the PIP4K2C protein exist and include NCBI accession nos. NP_001139730.1 (GI: 226371739); NP_001139731.1 (GI: 226371741); NP_001139732.1 (GI: 226371743); XP_005269209.1 (GI: 530400838); XP_011537049.1 (GI: 767975336); and/or XP_016875462.1 (GI: 1034581618). Any of these PIP4K2C proteins can be modified or targeted to reduce expression and/or reduce function thereof.

The PIP4K2A, PIP41K21, and PIP4K2C enzymes can have one or more amino acid differences compared to the sequences described herein. For example, subjects can have variant PIP4K2A, PIP4K2B, or PIP4K2C enzyme sequences with at least 80%, at least 85%, at least 90%, at least 95%, at least 96%, at least 97%, at least 99% amino acid sequence identity or similarity with any of the PIP4K2A, PIP4K2B, or PIP4K2C amino acid sequences described herein. Similarly, subjects can have PIP4K2A, PIP4K2B, or PIP4K2C RNA with one or more nucleotide differences compared to the PIP4K2A, PIP4K2B, or PIP4K2C nucleic acids described herein. For example, subjects can express a PIP4K2A, PIP4K2B, or PIP4K2C RNA at least 80%, at least 85%, at least 90%, at least 95%, at least 96%, at least 97%, at least 99% amino acid sequence identity or similarity with the PIP4K2A, PIP4K2B, or PIP4K2C nucleic acid sequences described herein.

The enzymes encoded by these two families of genes have sequence and structural similarities, but the activation loop of the PIP4Ks confers strict substrate selectivity for PI(5)P over PI(4)P (Kunz et al., 2001; Kunz et al., 2000a).

In settings where phosphoinositides have been quantified, PI(4)P and PI(4,5)P₂ each constitute between 30-50% of total cellular phosphoinositides. Whereas local levels of PI(4,5)P₂ can transiently drop when cells are stimulated with growth factors or hormones that activate PLC or PI3Ks, the total levels of PI(4,5)P₂ and PI(4)P remain remarkably constant. PI(4)P is over 100-fold more abundant than PI(5)P in cells, and it is generally assumed that most PI(4,5)P₂ in mammalian cells is generated from PI(4)P via PIP5Ks (Balla, 2013). In B-cell activation, transient recruitment of PIP5K1A is necessary for generation of PI(3,4,5)P₃ for signal transduction (Saito et al., 2003).

Because PI(5)P constitutes ˜1% of cellular phosphoinositides, it is uncertain if this lipid contributes substantially to total cellular PI(4,5)P₂, raising speculation that the function of the PIP4Ks is primarily to decrease the level of PI(5)P (Jones et al., 2006; Wilcox and Hinchliffe, 2008). Recent work by the inventors showed that conversion of PI(5)P to PI(4,5)P₂ by PIP4K2A/2B, likely on lysosomes, is needed to mediate fusion between autophagosomes and lysosomes (Lundquist et al., 2018). This study argued that while the PIP4Ks generate only a small fraction of cellular PI(4,5)P₂, the location of the PI(4,5)P₂ generated by these enzymes plays an important role in completion of autophagy. There is also evidence for pools of PIP4K2A/2B/2C in the plasma membrane, Golgi, and nucleus, so the PIP4K enzymes are likely to have many other functions beyond autophagy regulation (Bultsma et al., 2010; Jones et al., 2006; Mackey et al., 2014).

Overall, it is not clear how membrane lipid changes underlie the molecular mechanism by which the PIP4Ks regulate the PI3K pathway, and it is unknown whether one PIP4K isoform can compensate for others to suppress growth factor signal transduction.

As shown herein, PIP4Ks have distinct catalytic and non-catalytic functions in controlling cellular metabolism and it is the loss of catalytic-independent functions of PIP4Ks that underlie enhanced insulin signaling.

The roles of the PIP4K2A and PIP4K2B isoforms in autophagosome-lysosome fusion depends on the ability of these enzymes to convert PI(5)P to PI(4,5)P₂. However, experiments described herein revealed that the increased insulin sensitivity previously observed in Pip4k2b^{-/-} mice (Lamia et al., 2004) is due to loss of a non-catalytic function of PIP4K2B in suppressing the ability of PIP5Ks to produce PI(4,5)P₂ to be used as the substrate for PI 3-kinase. One confounding issue could be the increase in PI(5)P observed when PIP4Ks are depleted. Previous studies characterizing Ipgd, a bacterial 4-phosphatase of PI(4,5)P₂, have demonstrated that increasing PI(5)P levels prolongs Akt signaling (Carricaburu et al., 2003; Niebuhr et al., 2002; Pendaries et al., 2006). The model presented by Carricaburu et al, describes how increased cellular PI(5)P inhibits PI(3,4,5)P₃-specific phosphatase, thereby leading to an increase in PI(3,4,5)P₃ levels. This suggests that in certain settings, the catalytic activity of PIP4K modulates Akt signaling through control of PI(5)P levels.

However, the data provided herein shows that reconstitution with a kinase dead-PIP4K fully rescues PI(3,4,5)P₃ levels and Akt phosphorylation in response to insulin signaling, despite not reducing PI(5)P, indicating that the PIP4Ks have functions that are not currently understood in the art.

Also, as shown herein, PIP4Ks and PIP5Ks directly interact, and mutating five conserved amino acids of PIP4K2C can abrogate this interaction and prevent PIP5K suppression. A role in regulating PIP5K activity illuminates why PIP4Ks are highly abundant in cells, in excess of PIP5Ks which are thought to produce most of the PI(4,5)P₂. Because both PIP4Ks and PIP5Ks are bound to negatively charged membranes, these enzymes can also alter local membrane structures or recruit other enzymes to modify PIP5Ks to further modulate their activities.

The results shown herein indicate that methods and compositions that can degrade PIP4Ks may mitigate insulin resistance, and retard progression of type II diabetes. For example, specific PIP4K proteins can be degraded by linking them to E3 ligases (Bondeson and Crews, 2017).

The inventors initially had reservations about development of such drugs due to embryonic lethality of germline co-deletion of PIP4K2B/2C or PIP4K2A/2B (Emerling et al., 2013; Shim et al., 2016). Additionally, Emerling et al., 2013, and Lundquist et al., 2018 reported reduced viability of mouse embryonic fibroblasts upon loss of PIP4K2A/2B in cell lines with deficient p53 signaling (Emerling et al., 2013, Lundquist et al. 2018).

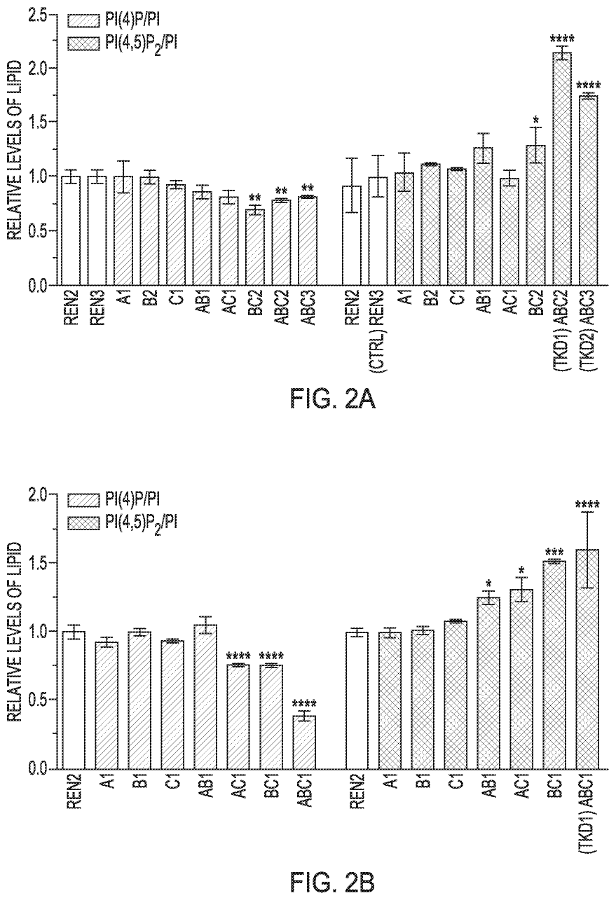

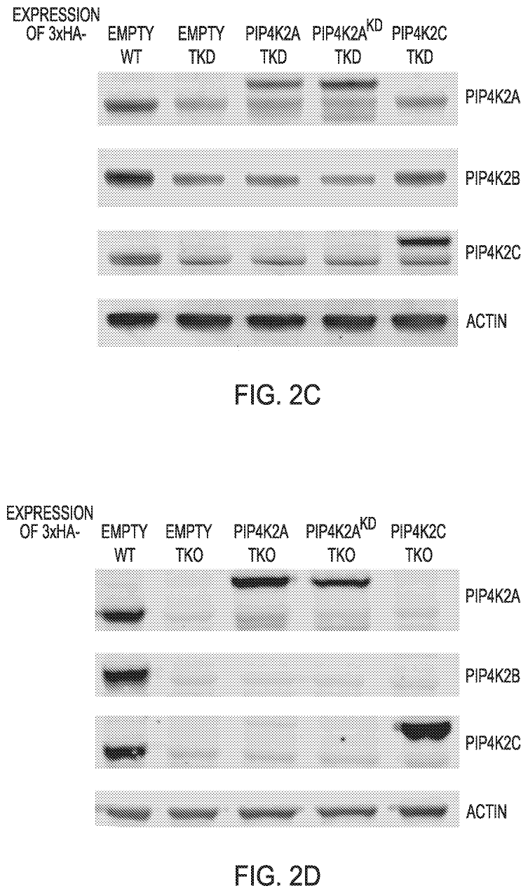

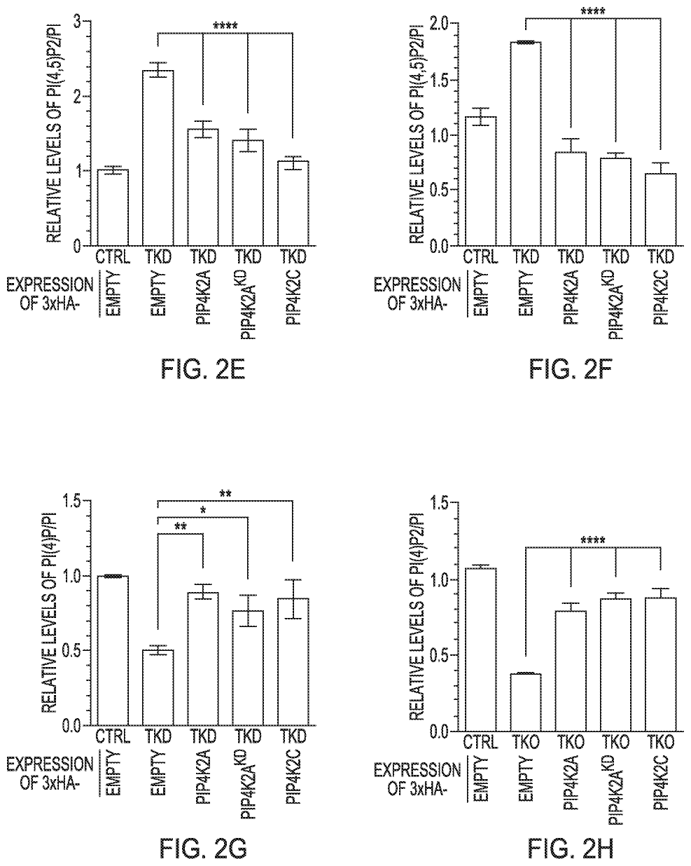

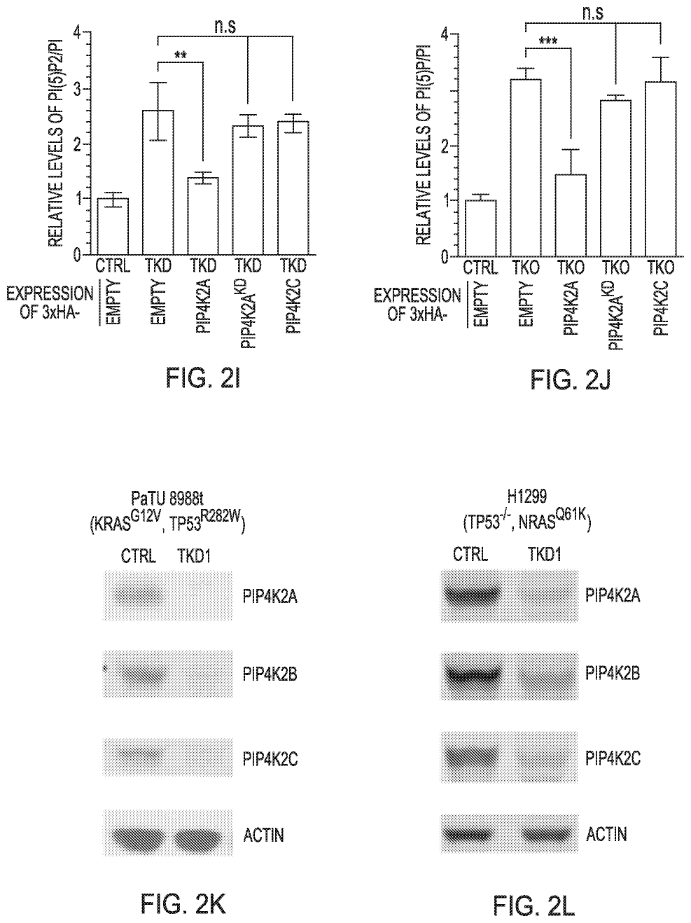

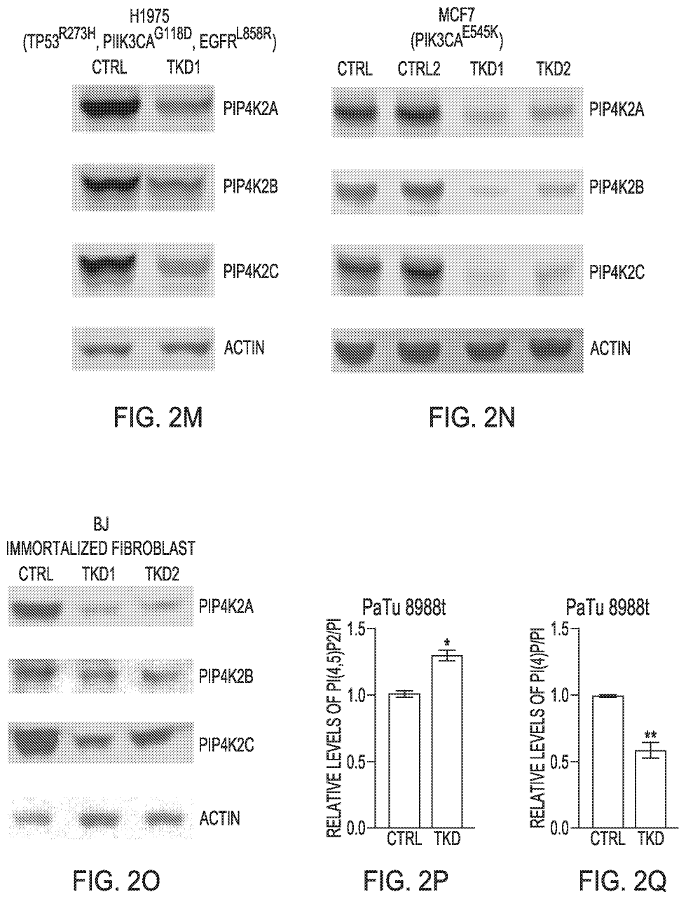

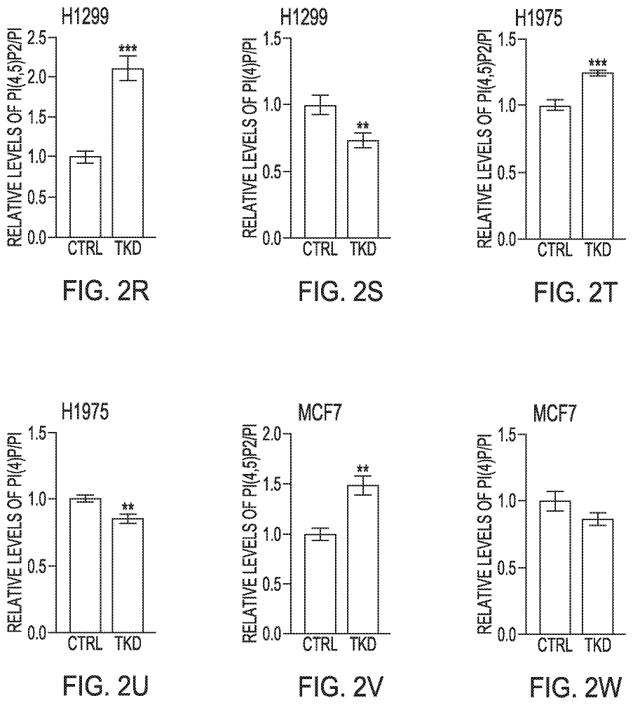

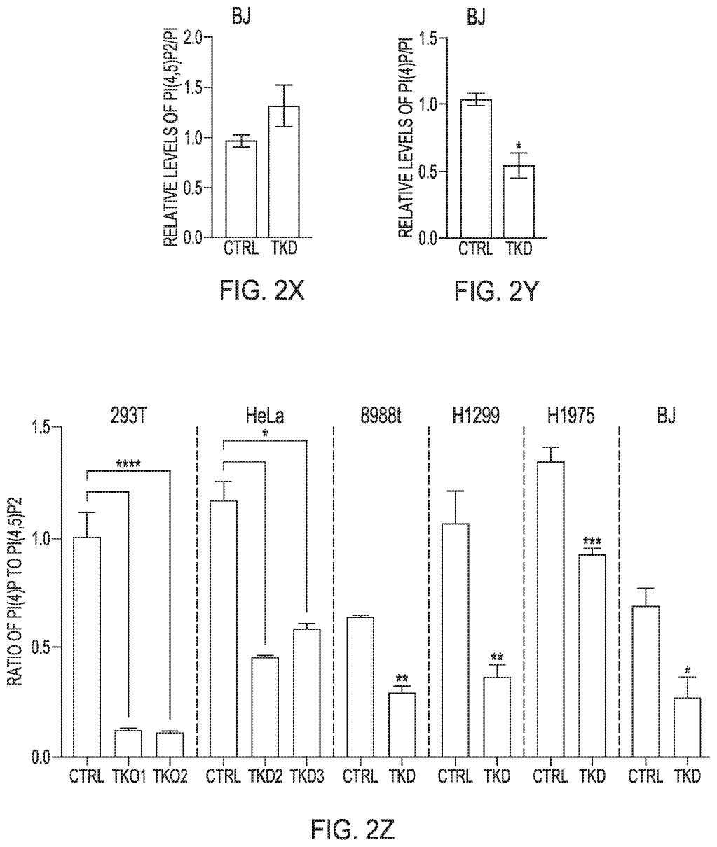

However, the results shown herein on a panel of cancer lines indicate that most cell lines do not require PIP4K isoforms for viability when grown in tissue culture with full nutrients (FIGS. 2K-2Z). Embryonic lethality from co-deletion of murine PIP4K isoforms may be attributed to developmental defects required for mammalian development or nutrient stress in the perinatal period (Emerling et al., 2013; Shim et al., 2016) and partial and/or tissue-specific targeted depletion may be well tolerated.

PIP4K family members repress the conversion of PI(4)P to PI(4,5)P₂ (and subsequently to PI(3,4,5)P₃) to limit PI3K pathway signaling. This occurs by a mechanism that does not require the catalytic activity of these enzymes, even while they directly catalyze the conversion of PI(5)P to PI(4,5)P₂ to promote autophagy. How did these two disparate functions come to reside in the same protein? One possibility is to ensure that levels of PI(4)P remain high at local sites of autophagosome-lysosome fusion. Several recent studies indicate that PI(4)P is required for autophagosome-lysosome fusion and that, in some cases, this PI(4)P is derived from an inositol 5-phosphatase acting on local pools of PI(4,5)P₂(De Leo et al., 2016; Wang et al., 2015). Thus, the PIP4Ks may be both producing the local PI(4,5)P₂ at the autophagosome-lysosome junction and ensuring that, upon conversion to PI(4)P, it is not converted back to PI(4,5)P₂ by a PIP5K. This model would ensure a unidirectional conversion of PI(5)P to PI(4)P and efficient autophagosome-lysosome fusion. The partial localization of PIP4K2C to the Golgi (Clarke et al., 2008) could also suppress conversion of Golgi PI(4)P to PI(4,5)P₂ to maintain high local concentration of the PI(4)P needed for vesicle trafficking at this location.

Given the global increase in PI(4,5)P₂ observed, there are undoubtedly multiple PI(4,5)P₂ mediated functions that will be affected. It will be interesting to characterize how accumulation of PI(4,5)P₂ in PIP4K-depleted cells affects cellular processes beyond growth factor signaling, such as cell adhesion, migration and/or calcium signaling. Further, it will be essential to examine how this change impacts the localization and levels of other phosphoinositides within cells.

In all, the findings shown herein highlight an unexpected and important separation of catalytic and non-catalytic functions in PIP4K family enzymes and indicate new avenues for intervention in enhancing insulin signaling, diabetes, metabolic syndrome, insulin resistance, obesity, cancer, autoimmune disease, and infection.

Degradation of PIP4K2A, PIP4K2B, and/or PIP4K2C Proteins

Although PIP4K2A, PIP4K2B, and/or PIP4K2C genetic knockdown or knockout can be used to reduce the cellular concentration or amount of these proteins, it can be preferable to post-translationally disrupt, degrade, or destabilize PIP4K2A, PIP4K2B, and/or PIP4K2C proteins. Targeting proteins directly, rather than via the DNA or mRNA molecules that encode them, is a more direct and rapid method for reducing the scaffolding function of PIP4K proteins. Hence, degradation can allow some PIP4K2A, PIP4K2B, and/or PIP4K2C catalytic function to proceed while reducing the non-catalytic functions such as scaffolding between PIP4K2A, PIP4K2B, and/or PIP4K2C proteins and other cellular proteins and structures.

The PIP4K2A, PIP4K2B, and/or PIP4K2C proteins can be directly disrupted, degraded, or destabilized in a variety of ways.

For example, PIP4K2A, PIP4K2B, and/or PIP4K2C proteins can be degraded by tagging endogenous PIP4K2A, PIP4K2B, and/or PIP4K2C proteins with an agent that signals cells to degrade the PIP4K2A, PIP4K2B. and/or PIP4K2C proteins.

One example of an agent that signals cells to degrade the PIP4K2A, PIP4K2B, and/or PIP4K2C proteins is an E3 ubiquitin ligase. Binding moieties can be used to link the degradation signal (e.g., E3 ubiquitin ligase) to the PIP4K2A. PIP4K2B, and/or PIP4K2C proteins. Such binding moieties can be antibodies, peptides, polysaccharides, lipids, or small molecules that bind specifically PIP4K2A, PIP4K2B, and/or PIP4K2C Antibody-bound PIP4K2A, PIP4K2B, and/or PIP4K2C proteins can be recognized by the cytosolic antibody receptor. TRIM21, which is an E3 ubiquitin ligase that binds with high affinity to the Fc domain of antibodies. Binding moieties can be linked to an E3 ubiquitin ligase to direct the E3 ubiquitin ligase to one or more PIP4K2A, PIP4K2B, and/or PIP4K2C protein. Any binding moiety for PIP4K2A, PIP4K2B, and/or PIP4K2C proteins can be adapted to directly or indirectly link or tag E3 ubiquitin ligase to the PIP4K2A, PIP4K2B, and/or PIP4K2C proteins.

Small molecules that bind PIP4K2A, PIP4K2B, and/or PIP4K2C proteins include those that are described, for example, in WO/2016/210291 and WO/2016/210296.

Methods for degradation or inhibition of PIP4K2A, PIP4K2B, and/or PIP4K2C can include introducing a complex to a subject where the complex is a protein with E3 ubiquitin ligase activity that is linked to a binding moiety for PIP4K2A, PIP4K2B, and/or PIP4K2C proteins to a subject or to a population of cells from a subject. Ubiquitination then occurs, and the PIP4K2A, PIP4K2B, and/or PIP4K2C proteins are degraded.

Methods for degradation or inhibition of PIP4K2A, PIP4K2B, and/or PIP4K2C can include inducing expression of an E3 ubiquitin ligase or introducing an exogenous an E3 ubiquitin ligase (e.g., TRIM21) expression system to a subject or into a population of cells from a subject, and introducing an antibody for PIP4K2A, PIP4K2B, and/or PIP4K2C proteins to a subject or to a population of cells from a subject. Ubiquitination then occurs followed by degradation of the antibody-bound PIP4K2A, PIP4K2B, and/or PIP4K2C proteins.

For example, at least four E3 ligases (i.e., MDM2, IAP, VHL, and cereblon) can be used as tags for degradation of PIP4K2A, PIP4K2B, and/or PIP4K2C proteins.

Mouse double minute 2 homolog (MDM2), also known as E3 ubiquitin-protein ligase Mdm2, is a nuclear-localized protein that in humans is encoded by the MDM2 gene. The encoded protein can promote tumor formation by targeting tumor suppressor proteins, such as p53, for proteasomal degradation. Mdm2 protein functions both as an E3 ubiquitin ligase that recognizes the N-terminal trans-activation domain (TAD) of the p53 tumor suppressor and an inhibitor of p53 transcriptional activation.

One example of sequence for a Homo sapiens E3 ubiquitin-protein ligase Mdm2 (isoform 2) is available as accession no. NP_001354919 XP_005268929 and shown below as SEQ ID NO:12.

| | |
|---|---|
| 1 | MCNTNMSVPT DGAVTTSQIP ASEQETLVRP KPLLLKLLKS |
| 41 | VGAQKDTYTM KEVLFYLGQY IMTKRLYDEK QQHIVYCSND |
| 81 | LLGDLFGVPS FSVKEHRKIY TMIYRNLVVV NQQESSDSGT |
| 121 | SVSENRCHLE GGSDQKDLVQ ELQEEKPSSS HLVSRPSTSS |
| 161 | RRRAISETEE NSDELSGERQ RKRHKSDSIS LSFDESLALC |
| 201 | VIREICCERS SSSESTGTPS NPDLDAGVSE HSGDWLDQDS |
| 241 | VSDQFSVEFE VESLDSEDYS LSEEGQELSD EDDEVYQVTV |
| 281 | YQAGESDTDS FEEDPEISLA DYWKCTSCNE MNPPLPSHCN |
| 321 | RCWALRENWL PEDKGKDKGE ISEKAKLENS TQAEEGFDVP |
| 361 | DCKKTIVNDS RESCVEENDD KITQASQSQE SEDYSQPSTS |
| 401 | SSIIYSSQED VKEFEREETQ DKEESVESSL PLNAIEPCVI |
| 441 | CQGRPKNGCI VHGKTGHLMA CFTCAKKLKK RNKPCPVCRQ |
| 481 | PIQMIVLTYF P |

Another example of a Homo sapiens E3 ubiquitin-protein ligase Mdm2 is available as accession no. CAP16727.1 and shown below as SEQ ID NO:13.

| | |
|---|---|
| 1 | MCNTNMSVPT DGAVTTSQIP ASEQETLVRP KPLLLKLLKS |
| 41 | VGAQKDTYTM KEFATKHRAK NIPV |

Another example of a Homo sapiens E3 ubiquitin-protein ligase Mdm2 is available as accession no. CAP16726.1 and shown below as SEQ ID NO:14.

| | |
|---|---|
| 1 | MCNTNMVPT DGAVTTSQIP ASEQETLVRP KPLLLKLLKS |
| 41 | VGAQKDTYTM KENHRTQVHL |

Another example of a Homo sapiens E3 ubiquitin-protein ligase Mdm2 is available as accession no. CAP16725.1 and shown below as SEQ ID NO:15.

| | |
|---|---|
| 1 | MCNTNMSVPT DGAVTTSQIP ASEQETLVRP KPLLLKLLKS |
| 41 | VGAQKDTYTM KEENIYHDLQ ELGSSQSAGR KFR |

Inhibitors of Apoptosis Protein (IAPs) are guardian ubiquitin ligases that keep classic pro-apoptotic proteins in check, and regulate not only caspases and apoptosis, but also modulates inflammatory signaling and immunity, copper homeostasis, mitogenic kinase signaling, cell proliferation, as well as cell invasion and metastasis. IAPs can act as direct caspase inhibitors and can directly bind to the active site pocket of CASP3 and CASP7 to obstruct substrate entry. IAPs can also inactivate CASP9 by keeping it in a monomeric, inactive state. IAP acts as an E3 ubiquitin-protein ligase regulating NF-kappa-B signaling and the target proteins for its E3 ubiquitin-protein ligase activity include: RIPK1, CASP3, CASP7, CASP8, CASP9, MAP3K2/MEKK2, DIABLO/SMAC, AIFM1, CCS and BIRC5/survivin. IAP plays a role in copper homeostasis by ubiquitinating COMMD1 and promoting its proteasomal degradation and can also function as E3 ubiquitin-protein ligase of the NEDD8 conjugation pathway, targeting effector caspases for neddylation and inactivation. IAP regulates the BMP signaling pathway and the SMAD and MAP3K7/TAK1 dependent pathways leading Lo NF-kappa-B and JNK activation.

One example of sequence for a Homo sapiens TAP E3 ubiquitin-protein ligase is available from the NCBI database as accession number P98170.2 and provided below as SEQ ID NO: 16.

| | |
|---|---|
| 1 | MTFNSFEGSK TCVPADINKE EEFVEEFNRL KTFANFPSGS |
| 41 | PVSASTLARA GFLYTGEGDT VRCFSCHAAV DRWQYGDSAV |
| 81 | GRHRKVSPNC RFINGFYLEN SATQSTNSGI QNGQYKVENY |
| 121 | LGSRDHFALD RPSETHADYL LRTGQVVDIS DTIYPRNPAM |
| 161 | YSEEARLKSF QNWPDYAHLT PRELASAGLY YTGIGDQVQC |
| 201 | FCCGGKLKNW EPCDRAWSEH RRHFPNCFFV LGRNLNIRSE |
| 241 | SDAVSSDRNF PNSTNLPRNP SMADYEARIF TFGTWIYSVN |
| 281 | KEQLARAGFY ALGEGDKVKC FHCGGGLTDW KPSEDPWEQH |
| 321 | AKWYPGCKYL LEQKGQEYIN NIHLTHSLEE CLVRTTEKTP |
| 361 | SLTRRIDDTI FQNPMVQEAI RMGFSFKDIK KIMEEKIQIS |
| 401 | GSNYKSLEVL VADLVNAQKD SMQDESSQTS LQKEISTEEQ |
| 441 | LRRLQEEKLC KICMDRNIAI VFVPCGHLVT CKQCAEAVDK |
| 481 | CPMCYTVITF KQKIFMS |

Another example of a Homo sapiens IAP E3 ubiquitin-protein ligase is available as accession no. Q13490.2 and shown below as SEQ ID NO: 17.

| | |
|---|---|
| 1 | MHKTASQRLF PGPSYQNIKS IMEDSTILSD WTNSNKQKMK |
| 41 | YDFSCELYRM STYSTFPAGV PVSERSLARA GFYYTGVNDK |
| 81 | VKCFCCGLML DNWKLGDSPI QKHKQLYPSC SFIQNLVSAS |
| 121 | LGSTSKNTSP MRNSFAHSLS PTLEHSSLFS GSYSSLSPNP |
| 161 | LNSRAVEDIS SSRTNPYSYA MSTEEARFLT YHMWPLTFLS |
| 201 | PSELARAGFY YIGPGDRVAC FACGGKLSNW EPKDDAMSEH |
| 241 | RRHFPNCPFL ENSLETLRFS ISNLSMQTHA ARMRTFMYWP |
| 281 | SSVPVQPEQL ASAGFYYVGR NDDVKCFCCD GGLRCWESGD |
| 321 | DPWVEHAKWF PRCEFLIRMK GQEFVDEIQG RYPHLLEQLL |
| 361 | STSDTTGEEN ADPPIIHFGP GESSSEDAVM MNTPVVKSAL |
| 401 | EMGFNRDLVK QTVQSKILTT GENYKTVNDI VSALLNAEDE |
| 441 | KREEEKEKQA EEMASDDLSL IRKNRMALFQ QLTCVLPILD |
| 481 | NLLKANVINK QEHDIIKQKT QIPLQARELI DTILVKGNAA |
| 521 | ANIFKNCLKE IDSTLYKNLF VDKNMKYIPT EDVSGLSLEE |
| 561 | QLRRLQEERT CKVCMDKEVS VVFIPCGHLV VCQECAPSLR |
| 601 | KCPICRGIIK GTVRTFLS |

Another example of a Homo sapiens IAP E3 ubiquitin-protein ligase is available as accession no. Q96CA5.2 and shown below as SEQ ID NO: 18.

| | |
|---|---|
| 1 | MGPKDSAKCL HRGPOPSHWA AGDGPTQERC GPRSLGSPVL |
| 41 | GLDTCRAWDH VDGQILGQLR PLTEEEEEEG AGATLSRGPA |
| 81 | FPGMGSEELR LASFYDWPLT AEVPPELLAA AGFFHTGHQD |
| 121 | KVRCFFCYGG LQSWKRGDDP WTEHAKWFPS CQFLLRSKGR |
| 161 | DFVHSVQETH SQLLGSWDPW EEPEDAAPVA PSVPASGYPE |
| 201 | LPTPRREVQS ESAQEPGGVS PAEAQRAWWV LEPPGARDVE |
| 241 | AQLRRLQEER TCKVCLDRAV SIVFVPCGHL VCAECAPGLQ |
| 281 | LCPICRAPVR SRVRTFLS |

The von Hippel-Lindau (VHL) tumor suppressor includes the substrate recognition subunit/E3 ligase complex VCB, which includes elongins B and C, and a complex including Cullin-2 and Rbx1. The primary substrate of VHL is Hypoxia Inducible Factor 1α (HIF-1α), a transcription factor that upregulates genes such as the pro-angiogenic growth factor VEGF and the red blood cell inducing cytokine erythropoietin in response to low oxygen levels.

One example of sequence for a Homo sapiens VHL E3 ubiquitin-protein ligase is available from the NCBI database as accession number NP_000542.1 and provided below as SEQ ID NO:19.

| | |
|---|---|
| 1 | MPRRAENWDE AEVGAEEAGV EEYGPEEDGG EESGAEESGP |
| 41 | EESGPEELGA EEEMEAGRPR PVLRSVNSRE PSQVIFCNRS |
| 81 | PRVVLPVWLN FDGEPQPYPT LPPGTGRRIH SYRGHLWLFR |
| 121 | DAGTHDGLLV NQTELFVPSL NVDGQPIFAN ITLPVYTLKE |
| 161 | RCLQVVRSLV KPENYRRLDI VRSLYEDLED HPNVQKDLER |
| 201 | LTQERIAHQR MGD |

Another example of a Homo sapiens VHL E3 ubiquitin-protein ligase is available as accession no. NP_937799.1 and shown below as SEQ ID NO:20.

| | |
|---|---|
| 1 | MPRRAENWDE AEVGAEEAGV EEYGPEEDGG EESGAEESGP |
| 41 | EESGPEELGA EEEMEAGRPR PVLRSVNSRE PSQVIFCNRS |
| 81 | PRVVLPVWLN FDGFPQPYPT LPPGTGRRIH SYRVYTLKER |
| 121 | CLQVVRSLVK PENYRRLDIV RSLYEDLEDH PNVQKDLERL |
| 161 | TQERIAHQRM GD |

Another example of a Homo sapiens VHL E3 ubiquitin-protein ligase is available as accession no. NP_001341652.1 and shown below as SEQ ID NO:21.

| | |
|---|---|
| 1 | MPRRAENWDE AEVGAEEAGV EEYGPEEDGG EESGAEESGP |
| 41 | EESGPEELGA EEEMEAGRPR PVLRSVNSRE PSQVIFCNRS |
| 81 | PRVVLPVWLN FDGEPQPYPT LPPGTGRRIH SYRVLMTPVG |
| 121 | QFCVVPALVE NTFLLGRLTD AKTGTSQGHV GAGRADRVWR |
| 161 | GKLTYLPAGR WRGCGCVVSV KEHFPEKEES RME |

Cereblon is a protein that in humans is encoded by the CRBN gene. Cereblon proteins are related to the Lon protease protein family. In mammals cereblon is found in the cytoplasm localized with a calcium channel membrane protein and is thought to play a role in brain development. Cereblon forms an E3 ubiquitin ligase complex with damaged DNA binding protein 1 (DDB1), Cullin-4A (CUL4A), and regulator of cullins 1 (ROC1). This complex ubiquitinates a number of other proteins. Through a mechanism which has not been completely elucidated, cereblon ubquitination of target proteins results in increased levels of fibroblast growth factor 8 (FGF8) and fibroblast growth factor 10 (FGF10). FGF8 in turn regulates a number of developmental processes, such as limb and auditory vesicle formation. The net result is that this ubiquitin ligase complex is important for limb outgrowth in embryos. In the absence of cereblon, DDB1 forms a complex with DDB2 that functions as a DNA damage-binding protein.

One example of sequence for a Homo sapiens cereblon E3 ubiquitin-protein ligase is available from the NCBI database as accession number NP_057386.2 and provided below as SEQ ID NO:22.

| | |
|---|---|
| 1 | MAGEGDQQDA AHNMGNHLPL LPAESEEEDE MEVEDQDSKE |
| 41 | AKKPNIINFD TSLPTSHTYL GADMEEFHGR TLHDDDSCQV |
| 81 | IPVLPQVMMI LIPGQTLPLQ LFHPQEVSMV RNLIQKDRTF |
| 121 | AVLAYSNVQE REAQFGTTAE IYAYREEQDF GIEIVKVKAI |
| 161 | GRQRFKVLEL RTQSDGIQQA KVQILPECVL PSTMSAVQLE |
| 201 | SLNKCQIFPS KPVSREDQCS YKWWQKYQKR KFHCANLTSW |
| 241 | PRWLYSLYDA ETLMDRIKKQ LREWDENLKD DSLPSNPIDF |
| 281 | SYRVAACLPI DDVLRIQLLK IGSAIQRLRC ELDIMNKCTS |
| 321 | LCCKQCQETE ITTKNEIFSL SLCGPMAAYV NPHGYVHETL |
| 361 | TVYKACNLNL IGRPSTEHSW FPGYAWTVAQ CKICASHIGW |
| 401 | KFTATKKDMS PQKFWGLTRS ALLPTIPDTE DEISPDKVIL |
| 441 | CL |

Another example of a Homo sapiens cereblon E3 ubiquitin-protein ligase is available as accession no. NP_001166953.1 and shown below as SEQ ID NO:23.

| | |
|---|---|
| 1 | MAGEGDQQDA AHNMGNHLPL LPESEEEDEM EVEDQDSKEA |
| 41 | KKPNIINFDT SLPTSHTYLG ADMEEFHGRT LHDDDSCQVI |
| 81 | PVLPQVMMIL IPGQTLPLQL FHPQEVSMVR NLIQKDRTFA |
| 121 | VLAYSNVQER EAQFGTTAEI YAYREEQDFG IEIVKVKAIG |
| 161 | RQRFKVLELR TQSDGIQQAK VQILPECVLP STMSAVQLES |
| 201 | LNKCQIFPSK PVSREDQCSY KWWQKYQKRK FHCANLTSWP |
| 241 | RWLYSLYDAE TLMDRIKKQL REWDENLKDD SLPSNPIDFS |
| 281 | YRVAACLPID DVLRTQLLKI GSAIQRLRCE LDIMNKCTSL |
| 321 | CCKQCQETEI TTKNEIFSLS LCGPMAAYVN PHGYVHETLT |
| 361 | VYKACNLNLI GRPSTEHSWF PGYAWTVAQC KICASHIGWK |
| 401 | FTATKKDMSP QKFWGLTRSA LLPTIPDTED EISPDKVILC |
| 441 | L |

Another example of a Homo sapiens cereblon E3 ubiquitin-protein ligase is available as accession no. XP_005265259.1 and shown below as SEQ ID NO:24.

| | |
|---|---|
| 1 | MEEFHGRTLH DDDSCQVIPV LPQVMMILIP GQTLPLQLFH |
| 41 | PQEVSMVRNL IQKDRTFAVL AYSNVQEREA QFGTTAEIYA |
| 81 | YREEQDFGIE IVKVKAIGRQ RFKVLELRTQ SDGIQQAKVQ |
| 121 | ILPECVLPST MSAVQLESLN KCQIFPSKPV SREDQCSYKW |
| 161 | WQKYQKRKFH CANLTSWPRW LYSLYDAETL MDRIKKQLRE |
| 201 | WDENLKDDSL PSNPIDFSYR VAACLPIDDV LRIQLLKIGS |
| 241 | AIQRLRCELD IMNKCTSLCC KQCQETEITT KNEIFSLSLC |
| 281 | GPMAAYVNPH GYVHETLTVY KACNLNLIGR PSTEHSWFPG |
| 321 | YAWTVAQCKI CASHIGWKFT ATKKDMSPQK FWGLTRSALL |
| 361 | PTIPDTEDEI SPDKVILCL |

Another example of a Homo sapiens cereblon E3 ubiquitin-protein ligase is available as accession no. XP_011532093.1 and shown below as SEQ ID NO:25.

| | |
|---|---|
| 1 | MAGEGDQQDA AHNMGNHLPL LPAESEEEDE MEVEDQDSKE |
| 41 | AKKPNIINFD TSLPTSHTYL GADMEEFHGR TLHDDDSCQV |
| 81 | IPVLPQVMMI LIPGQTLPLQ LFHPQEVSMV RNLIQKDRTF |
| 121 | AVLAYSNVQE REAQFGTTAE IYAYREEQDF GIEIVKVKAI |
| 161 | GRQRFKVLEL RTQSDGIQQA KVQILPECVL PSTMSAVQLE |
| 201 | SLNKCQIFPS KPVSREDQCS YKWWQKYQKR KFHCANLTSW |
| 241 | PRWLYSLYDA ETLMDRIKKQ LREWDENLKD DSLPSNPIDF |
| 281 | SYRVAACLPI DDVLRIQLLK IGSAIQRLRC ELDIMNKCTS |
| 321 | LCCKQCQETE ITTKNEIFRY AWTVAQCKIC ASHIGWKFTA |
| 361 | TKKDMSPQKF WGLTRSALLP TIPDTEDEIS PDKVILCL |

As described above, antibody-bound PIP4K2A, PIP4K2B, and/or PIP4K2C proteins can be recognized by the cytosolic antibody receptor, TRIM21, which is an E3 ubiquitin ligase that binds with high affinity to the Fc domain of antibodies. Treatment with an antibody that binds specification to a PIP4K2A, PIP4K2B, and/or PIP4K2C protein, either with or before administering or inducing the expression of TRIM21 can lead to degradation of the PIP4K2A, PIP4K2B, and/or PIP4K2C protein.

One example of sequence for a Homo sapiens E3 ubiquitin-protein ligase TRIM21 polypeptide sequence is available from the NCBI database as accession number NP_003132.2 and provided below as SEQ ID NO:26.

| | |
|---|---|
| 1 | MASAARLTMM WEEVTCPICL DPFVEPVSIE CGHSFCQECI |
| 41 | SQVGKGGGSV CPVCRQRFLL KNLRPNRQLA NMVNNLKEIS |
| 81 | QEAREGTQGE RCAVHGERLH LFCEKDGKAL CWVCAQSRKH |
| 121 | RDHAMVPLEE AAQEYQEKLQ VALGELRRKQ ELAEKLEVEI |
| 161 | AIKRADWKKT VETQKSRIHA EFVQQKNFLV EEEQRQLQEL |
| 201 | EKDEREQLRI LGEKEAKLAQ QSQALQELIS ELDRRCHSSA |
| 241 | LELLQEVIIV LERSESWNLK DLDITSPELR SVCHVPGLKK |
| 281 | MLRTCAVHIT LDPDTANPWL ILSEDRRQVR LGDTQQSIPG |
| 321 | NEERFDSYPM VLGAQHFHSG KHYWEVDVTG KEAWDLGVCR |
| 361 | DSVRRKGHFL LSSKSGFWTI WLWNKQKYEA GTYPQTPLHL |
| 401 | QVPPCQVGIF LDYEAGMVSF YNITDHGSLI YSFSECAFTG |
| 441 | PLRPFFSPGF NDGGKNTAPL TLCPLNIGSQ GSTDY |

Similarly, a PROteolysis-TArgeting Chimeras (PROTACs) system can be used to tag one or more of the PIP4K2A, PIP4K2B, and/or PIP4K2C for selective degradation. The PROTAC systems include a ligand to the target PIP4K2A, PIP4K2B, and/or PIP4K2C protein, a ligand to the E3 ubiquitin ligase, and a linker connecting the two ligands. See, e.g., Bondeson & Crew, Annu Rev Pharmacol Toxicol. 57: 107-123 (2017).

Fragments of E3 ubiquitin ligases that can induce ubiquitination can also be used. For example, the E3 ubiquitin ligases include those that have at least 20, at least 22, at least 25, at least 27, at least 30, at least 35, at least 40, at least 50 of the same amino acids as an E3 ubiquitin ligases. The identical amino acids can be distributed throughout the E3 ubiquitin ligases and need not be contiguous but are present in homologous positions.

The at least 70%, 80%, 90%, 95%, 96%, 97%, 98%, 99% or more than 99% sequence identity to any of the E3 ubiquitin ligases described herein, or at least 70%, 80%, 90%, 95%, 96%, 97%, 98%, 99% or more than 99% sequence identity to a fragment of an E3 ubiquitin ligase that has at least 20, at least 22, at least 25, at least 27, at least 30, at least 35, at least 40, at least 50 amino acids.

Expression vectors that include a nucleic acid segment that encodes any of these E3 ubiquitin ligase proteins can in some cases be used to increase expression of the E3 ubiquitin ligase proteins.

Antibodies that Bind PIP4K

In some cases, isolated antibodies that bind specifically to PIP4K can be used in the compositions and methods described herein. Such antibodies may be monoclonal antibodies. In some cases, the antibodies can be polyclonal antibodies. Such antibodies may also be humanized or fully human antibodies. The antibodies can exhibit one or more desirable functional properties, such as high affinity or specific binding to PIP4K.

Methods and compositions described herein can include PIP4K antibodies, or a combination of PIP4K antibodies with agents that induce or mediate the degradation of PIP4K2A, PIP4K2B, and/or PIP4K2C.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VI) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g. an epitope or a domain of PIP4K). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody,” as used herein, is intended to refer to an antibody that is substantially free of other antibodies having different antigenic specificities (e.g., an isolated antibody that specifically binds PIP4K is substantially free of antibodies that specifically bind antigens other than PIP4K. In some cases, the antibodies ay however, have cross-reactivity to other antigens, such as PIP4K protein variants or PIP4K from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{L} and V_{H} regions of the recombinant antibodies are sequences that, while derived from and related to human germline V_{L} and V_{H} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to a PIP4K” is intended to refer to an antibody that binds to a specific type of PIP4K with a K_{D} of 1×10⁻⁷ M or less, more preferably 5×10⁻⁸ M or less, more preferably 1×10⁻⁸ M or less, more preferably 5×10⁻⁹ M or less, even more preferably between 1×10⁻⁸ M and 1×10⁻¹⁰ M or less.

The term “K_{assoc}” or “Kₐ,” as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D},” as used herein, is intended to refer to the dissociation constant, which is obtained from the ratio of K_{dis} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore™ system.

The antibodies of the invention are characterized by particular functional features or properties of the antibodies. For example, the antibodies bind specifically to human PIP4K. Preferably, an antibody of the invention binds to PIP4K with high affinity, for example with a K_{D} of 1×10⁻⁷ M or less (e.g., less than 1×10⁻⁸ M or less than 1×10⁻⁹ M). The antibodies can exhibit one or more of the following characteristics:

(a) binds to one or more human PIP4K with a K_{D} of 1×10⁷ M or less;

(b) facilitates degradation of one or more types of PIP4K proteins;

(c) enhances insulin signaling or reduces insulin resistance;

(d) reduces the symptoms or severity of diabetes;

(e) enhances immune responses;

(f) reduces cancer cell growth or cancer progression; or

(g) a combination thereof.

Assays to evaluate the binding ability of the antibodies toward PIP4K can be used, including for example, ELISAs, Western blots and RIAs. The binding kinetics (e.g., binding affinity) of the antibodies also can be assessed by standard assays known in the art, such as by Biacore™ analysis.

Given that the subject antibody preparations can bind to PIP4K, the V_{L} and V_{H} sequences can be “mixed and matched” to create other binding molecules that bind to PIP4K. The binding properties of such “mixed and matched” antibodies can be tested using the binding assays (e.g., ELISAs). When V_{L} and V_{H} chains are mixed and matched, a V_{H} sequence from a particular V_{H}/V_{L} pairing can be replaced with a structurally similar V_{H} sequence. Likewise, preferably a V_{L} sequence from a particular V_{H}/V_{L} pairing is replaced with a structurally similar V_{L} sequence.

Accordingly, in one aspect, the invention provides an isolated monoclonal antibody, or antigen binding portion thereof comprising:

(a) a heavy chain variable region comprising an amino acid sequence; and

(b) a light chain variable region comprising an amino acid sequence;

wherein the antibody specifically binds PIP4K.

In some cases, the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, for example, Klimka et al., British J. of Cancer 83(2):252-260 (2000) (describing the production of a humanized anti-CD30 antibody using only the heavy chain variable domain CDR3 of murine anti-CD30 antibody Ki-4); Beiboer et al., J. Mol. Biol. 296:833-849 (2000) (describing recombinant epithelial glycoprotein-2 (EGP-2) antibodies using only the heavy chain CDR3 sequence of the parental murine MOC-31 anti-EGP-2 antibody); Rader et al., Proc. Natl. Acad. Sci. U.S.A. 95:8910-8915 (1998) (describing a panel of humanized anti-integrin alphaᵥbeta₃ antibodies using a heavy and light chain variable CDR3 domain. Hence, in some cases a mixed and matched antibody or a humanized antibody contains a CDR3 antigen binding domain that is specific for PIP4K.

Nucleic Adds that Inhibit PIP4K

Various inhibitors of PIP4K function can be employed in the compositions and methods described herein. For example, one type of PIP4K inhibitor can be an inhibitory nucleic acid. The expression or translation of an endogenous PIP4K can be inhibited, for example, by use of an inhibitory nucleic acid that specifically binds to an endogenous (target) nucleic acid that encodes PIP4K.

An inhibitory nucleic acid can have at least one segment that will hybridize to PIP4K nucleic acid under intracellular or stringent conditions. The inhibitory nucleic acid can reduce expression of a nucleic acid encoding PIP4K. An inhibitory nucleic acid may hybridize to a genomic DNA, a messenger RNA, or a combination thereof. An inhibitory nucleic acid may be incorporated into a plasmid vector or viral DNA. It may be single stranded or double stranded, circular or linear.

An inhibitory nucleic acid is a polymer of ribose nucleotides or deoxyribose nucleotides having more than 13 nucleotides in length. An inhibitory nucleic acid may include naturally-occurring nucleotides; synthetic, modified, or pseudo-nucleotides such as phosphorothiolates; as well as nucleotides having a detectable label such as P³², biotin or digoxigenin. An inhibitory nucleic acid can reduce the expression and/or activity of a PIP4K nucleic acid. Such an inhibitory nucleic acid may be completely complementary to a segment of PIP4K nucleic acid (e.g., to a PIP4K mRNA). Alternatively, some variability is permitted in the inhibitory nucleic acid sequences relative to PIP4K sequences. For example, the PIP4K nucleic acids or PIP4K proteins can have at least 85% sequence identity and/or complementary, or at least 90% sequence identity and/or complementary, or at least 95% sequence identity and/or complementary, or at least 96% sequence identity and/or complementary, or at least 97% sequence identity and/or complementary, or at least 98% sequence identity and/or complementary, or at least 99% sequence identity and/or complementary to the target PIP4K nucleic acid.

An inhibitory nucleic acid can hybridize to a PIP4K nucleic acid under intracellular conditions or under stringent hybridization conditions and is sufficient to inhibit expression of a PIP4K nucleic acid. Intracellular conditions refer to conditions such as temperature, pH and salt concentrations typically found inside a cell, e.g. an animal or mammalian cell. One example of such an animal or mammalian cell is a muscle, liver, fat, or pancreatic cell such as an islets of Langerhans cell, a pancreatic progenitor cell or a pancreatic beta cell. However, because insulin resistance typically occurs when muscle, fat, and liver cells do not respond well to insulin and can't easily take up glucose, the cellular target may be non-pancreatic cells (e.g., muscle, liver, fat, nervous, lymphocytes, etc.). Generally, stringent hybridization conditions are selected to be about 5° C. lower than the thermal melting point (Tm) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory oligonucleotides that comprise, for example, 2, 3, 4, or 5 or more stretches of contiguous nucleotides that are precisely complementary to a PIP4K coding or flanking sequence, can each be separated by a stretch of contiguous nucleotides that are not complementary to adjacent coding sequences, and such an inhibitory nucleic acid can still inhibit the function of a PIP4K nucleic acid. In general, each stretch of contiguous nucleotides is at least 4, 5, 6, 7, or 8 or more nucleotides in length. Non-complementary intervening sequences may be 1, 2, 3, or 4 nucleotides in length.

One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a short hairpin RNA, a small interfering RNA, a ribozyme or an antisense nucleic acid molecule.

The inhibitory nucleic acid molecule may be single or double stranded (e.g. a small interfering RNA (siRNA)) and may function in an enzyme-dependent manner or by steric blocking. Inhibitory nucleic acid molecules that function in an enzyme-dependent manner include forms dependent on RNase H activity to degrade target mRNA. These include single-stranded DNA, RNA, and phosphorothioate molecules, as well as the double-stranded RNAi/siRNA system that involves target mRNA recognition through sense-antisense strand pairing followed by degradation of the target mRNA by the RNA-induced silencing complex. Steric blocking inhibitory nucleic acids, which are RNase-H independent, interfere with gene expression or other mRNA-dependent cellular processes by binding to a target mRNA and getting in the way of other processes. Steric blocking inhibitory nucleic acids include 2′-O alkyl (usually in chimeras with RNase-H dependent antisense), peptide nucleic acid (PNA), locked nucleic acid (LNA) and morpholino antisense.

Small interfering RNAs, for example, may be used to specifically reduce PIP4K translation such that translation of the encoded polypeptide is reduced. SiRNAs mediate post-transcriptional gene silencing in a sequence-specific manner. See, for example, website at invitrogen.com/site/us/en/home/Products-and-Services/Applications/rnai.html. Once incorporated into an RNA-induced silencing complex, siRNA mediate cleavage of the homologous endogenous mRNA transcript by guiding the complex to the homologous mRNA transcript, which is then cleaved by the complex. The siRNA may be homologous to any region of the PIP4K mRNA transcript. The region of homology may be 30 nucleotides or less in length, such as less than 25 nucleotides, or for example about 21 to 23 nucleotides in length. SiRNA is typically double stranded and may have two-nucleotide 3′ overhangs, for example, 3′ overhanging UU dinucleotides. Methods for designing siRNAs are available, see, for example, Elbashir et al. Nature 411: 494-498 (2001); Harborth et al. Antisense Nucleic Acid Drug Dev. 13: 83-106 (2003).

The pSuppressorNeo vector for expressing hairpin siRNA, commercially available from IMGENEX (San Diego, Calif.), can be used to make siRNA or shRNA for inhibiting PIP4K expression. The construction of the siRNA or shRNA expression plasmid involves the selection of the target region of the mRNA, which can be a trial-and-error process. However, Elbashir et al. have provided guidelines that appear to work ˜80% of the time. Elbashir, S. M., et al., Analysis of gene function in somatic mammalian cells using small interfering RNAs. Methods, 2002. 26(2): p. 199-213. Accordingly, for synthesis of synthetic siRNA or shRNA, a target region may be selected preferably 50 to 100 nucleotides downstream of the start codon. The 5′ and 3′ untranslated regions and regions close to the start codon should be avoided as these may be richer in regulatory protein binding sites. As siRNA can begin with AA, have 3′ UU overhangs for both the sense and antisense siRNA strands, and have an approximate 50% G/C content. An example of a sequence for a synthetic siRNA or shRNA is 5′-AA(N19)UU, where N is any nucleotide in the mRNA sequence and should be approximately 50% G-C content. The selected sequence(s) can be compared to others in the human genome database to minimize homology to other known coding sequences (e.g., by Blast search, for example, through the NCBI website).

SiRNAs may be chemically synthesized, created by in vitro transcription, or expressed from an siRNA expression vector or a PCR expression cassette. See, e.g., website at invitrogen.com/site/us/en/home/Products-and-Services/Applications/rnai.html. When an siRNA is expressed from an expression vector or a PCR expression cassette, the insert encoding the siRNA may be expressed as an RNA transcript that folds into an siRNA hairpin or a shRNA. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, or about 3 to 23 nucleotides in length, and may include various nucleotide sequences including for example, AUG, CCC, UUCG, CCACC, CTCGAG, AAGCULU, and CCACACC. SiRNAs also may be produced in vivo by cleavage of double-stranded RNA introduced directly or via a transgene or virus. Amplification by an RNA-dependent RNA polymerase may occur in some organisms.

An inhibitory nucleic acid such as a short hairpin RNA siRNA or an antisense oligonucleotide may be prepared using methods such as by expression from an expression vector or expression cassette that includes the sequence of the inhibitory nucleic acid. Alternatively, it may be prepared by chemical synthesis using naturally-occurring nucleotides, modified nucleotides or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the inhibitory nucleic acid and the target PIP4K nucleic acid.

### Genomic Modification to Reduce PIP4K

In some cases, PIP4K expression of functioning can be reduced by genomic modification of one or more PIP4K genes.

Non-limiting examples of methods of introducing a modification into the genome of a cell can include use of microinjection, viral delivery, recombinase technologies, homologous recombination, TALENS, CRISPR, and/or ZFN, see, e.g. Clark and Whitelaw Nature Reviews Genetics 4:825-833 (2003); which is incorporated by reference herein in its entirety.

For example, nucleases such as zinc finger nucleases (ZFNs), transcription activator like effector nucleases (TALENs), and/or meganucleases can be employed with a guide nucleic acid that allows the nuclease to target the genomic PIP4K site(s). In some cases, a targeting vector can be used to introduce a deletion or modification of one or more genomic PIP4K site(s).

A “targeting vector” is a vector generally has a 5′ flanking region and a 3′ flanking region homologous to segments of the gene of interest. The 5′ flanking region and a 3′ flanking region can surround a DNA sequence comprising a modification and/or a foreign DNA sequence to be inserted into the gene. For example, the foreign DNA sequence may encode a selectable marker. In some cases, the targeting vector does not comprise a selectable marker, but such a selectable marker can facilitate identification and selection of cells with desirable mutations. Examples of suitable selectable markers include antibiotics resistance genes such as chloramphenicol resistance, gentamycin resistance, kanamycin resistance, spectinomycin resistance (SpecR), neomycin resistance gene (NEO), and/or the hygromycin β-phosphotransferase genes. The 5′ flanking region and the 3′ flanking region can be homologous to regions within the gene, or to regions flanking the gene to be deleted, modified, or replaced with the unrelated DNA sequence. The targeting vector is contacted with the native gene of interest in vivo (e.g., within the cell) under conditions that favor homologous recombination. For example, the cell can be contacted with the targeting vector under conditions that result in transformation of the cyanobacterial cell(s) with the targeting vector.

A typical targeting vector contains nucleic acid fragments of not less than about 0.1 kb nor more than about 10.0 kb from both the 5′ and the 3′ ends of the genomic locus which encodes the gene to be modified (e.g. the genomic PIP4K site(s)). These two fragments are separated by an intervening fragment of nucleic acid which encodes the modification to be introduced. When the resulting construct recombines homologously with the chromosome at this locus, it results in the introduction of the modification, e.g. a deletion of a portion of the genomic PIP4K site(s), replacement of the genomic PIP4K promoter or coding region site(s), or the insertion of non-conserved codon or a stop codon.

In some cases, a Cas9/CRISPR system can be used to create a modification in genomic PIP4K that reduces the expression or functioning of the PIP4K gene products. Clustered regularly interspaced short palindromic repeats (CRISPR)/CRISPR-associated (Cas) systems are useful for, e.g. RNA-programmable genome editing (see e.g., Marraffini and Sontheimer. Nature Reviews Genetics 11: 181-190 (2010); Sorek et al. Nature Reviews Microbiology 2008 6: 181-6; Karginov and Hannon. Mol Cell 2010 1:7-19; Hale et al. Mol Cell 2010:45:292-302; Jinek et al. Science 2012 337:815-820; Bikard and Marraffini Cuff Opin Immunol 2012 24:15-20; Bikard et al. Cell Host & Microbe 2012 12: 177-186: all of which are incorporated by reference herein in their entireties). A CRISPR guide RNA can be used that can target a Cas enzyme to the desired location in the genome, where it generates a double strand break. This technique is described, for example, by Mali et al. Science 2013 339:823-6: which is incorporated by reference herein in its entirety. Kits for the design and use of CRISPR-mediated genome editing are commercially available, e.g. the PRECISION X CAS9 SMART NUCLEASE™ System (Cat No. CAS900A-1) from System Biosciences, Mountain View, Calif.

In other cases, a cre-lox recombination system of bacteriophage P1, described by Abremski et al. 1983. Cell 32:1301 (1983). Sternberg et al., Cold Spring Harbor Symposia on Quantitative Biology, Vol. XLV 297 (1981) and others, can be used to promote recombination and alteration of the genomic PIP4K site(s). The cre-lox system utilizes the cre recombinase isolated from bacteriophage P1 in conjunction with the DNA sequences that the recombinase recognizes (termed lox sites). This recombination system has been effective for achieving recombination in plant cells (see, e.g., U.S. Pat. No. 5,658,772), animal cells (U.S. Pat. Nos. 4,959,317 and 5,801,030), and in viral vectors (Hardy et al., J. Virology 71:1842 (1997).

The genomic mutations so incorporated can alter one or more amino acids in the encoded PIP4K gene products. For example, genomic sites modified so that in the encoded PIP4K protein is more prone to degradation, or is less stable, so that the half-life of such protein(s) is reduced. In another example, genomic sites can be modified so that at least one amino acid of a PIP4K polypeptide is deleted or mutated to reduce the enzymatic activity at least one type of PIP4K. In some cases, a conserved amino acid or a conserved domain of the PIP4K polypeptide is modified. For example, a conserved amino acid or several amino acids in a conserved domain of the PIP4K polypeptide can be replaced with one or more amino acids having physical and/or chemical properties that are different from the conserved amino acid(s). For example, to change the physical and/or chemical properties of the conserved amino acid(s), the conserved amino acid(s) can be deleted or replaced by amino acid(s) of another class, where the classes are identified in the following Table 6.

TABLE 6

| Classification | Genetically Encoded |
|---|---|
| Hydrophobic | A, G, F, I, L, M, P, V, W |
| Aromatic | F, Y, W |
| Apolar | M, G, P |
| Aliphatic | A, V, L, I |
| Hydrophilic | C, D, E, H, K, N, Q, R, S, T, Y |
| Acidic | D, E |
| Basic | H, K, R |
| Polar | Q, N, S, T, Y |
| Cysteine-Like | C |

Different types of amino acids can be employed in the PIP4K polypeptide. Examples are shown in Table 7.

TABLE 7

| | One-Letter | Common |
|---|---|---|
| Amino Acid | Symbol | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| β-Alanine | | bAla |
| N-Methylglycine | | MeGly |
| (sarcosine) | | |
| Ornithine | | Orn |
| Norleucine | | Nle |
| Penicillamine | | Pen |
| Homoarginine | | hArg |
| N-methylvaline | | MeVal |
| Homocysteine | | hCys |
| Homoserine | | hSer |

Such genomic modifications can reduce the expression or functioning of PIP4K gene products by at least 10%, or at least 15%, or at least 20%, or at least 25%, or at least 30%, or at least 35%, or at least 40%, or at least 45%, or at least 50% compared to the unmodified PIP4K gene product expression or functioning.

### Insulin Resistance

As described herein, depletion or degradation of PIP4Ks, the particularly PIP4K2B protein, is useful for treatment of insulin resistance.

Insulin stimulates conversion of phosphatidylinositol-4,5-bisphosphate (PI(4,5)P₂) to phosphatidylinositol-3,4,5-trisphosphate (PI(3,4,5)P₃), which mediates downstream cellular responses. PI(4,5)P₂ is produced by phosphatidylinositol-4-phosphate 5-kinases (PIP5Ks) and by phosphatidylinositol-5-phosphate 4-kinases (PIP4Ks). As shown herein, loss of PIP4Ks (PIP4K2A, PIP4K2B and PIP4K2C) in vitro results in a paradoxical increase in PI(4,5)P₂ and concomitant increase in insulin-stimulated production of PI(3,4,5)P₃. Surprisingly, reintroduction of either wild-type or kinase-dead mutants of the PIP4Ks restored cellular PI(4,5)P₂ levels and insulin stimulation of the PI3K pathway, indicating a catalytic-independent role of PIP4Ks in regulating PI(4,5)P₂ levels. These effects are explained by an increase in PIP5K activity upon deletion of PIP4Ks, which normally suppresses PIP5K activity through a direct binding interaction mediated by PIP4Ks' N-terminal motif VMLXPDD (SEQ ID NO:5, where X is any amino acid). The experiments described herein show that PIP4Ks have an allosteric function in suppressing PIP5K-mediated PI(4,5)P₂ synthesis and insulin-dependent conversion to PI(3,4,5)P₃. The methods and compositions described herein that deplete PIP4K enzymes are useful for enhancing insulin signaling.

Methods of Identifying Agents that can Enhance or Treat Insulin Signaling

The invention further provides screening assays that are useful for generating or identifying therapeutic agents for prevention and treatment of diabetes, enhancing insulin signaling, reducing insulin resistance, and assays for generating or identifying agents that inhibit PIP4K. In particular, PIP4K may be used in a variety of assays for identifying factors that enhance insulin signaling, reduce insulin resistance, modulate PIP5K-mediated PI(4,5)P₂ synthesis, increase in PIP5K activity, and increase insulin-dependent conversion to PI(3,4,5)P₃.

For example, in one embodiment, the invention relates to a method of identifying a therapeutic agent that can inhibit PIP4K. Such a method can be an in vitro or in vivo method.

The methods can involve use of an animal model for diabetes or insulin signaling. For example, a method of identifying a therapeutic agent can involve administering a test agent to an experimental animal that expresses PIP4K in muscle, liver, fat, nervous, or pancreatic cells and observing whether one or more symptoms of diabetes, insulin resistance, or insulin signaling are improved in the experimental animal. The cells can include muscle cells, liver cells, lymphocytes, fat cells, nervous cells, or a combination thereof. In some embodiments, the method also includes comparing the Akt, phosphoinositide, PI(3,4,5)P₃, PI(4,5)P₂ activity or levels compared to a control experimental animal has not been administered the test agent or a control experimental animal that has also been administered the test agent but that does not express PIP4K.

Examples of experimental animals that can be employed include mice, rats, dogs, goats, monkeys, and chimpanzees. In general, any experimental animal can be employed so long as it is susceptible to diabetes, or insulin insensitivity. One type of mouse strain that can be used as a model of diabetes are NOD.Cg-Prkdcscid Il2rgtm1Wj1/SzJ (NSG) mice (e.g., from Jackson Laboratory, Bar Harbor, Me.) that are injected intraperitoneally with Streptozocin (STZ; e.g., from Sigma-Aldrich, St. Louis, Mo.) or other mouse strains described in the Examples.

Dosages of known and newly identified therapeutic agents can also be determined by use of such methods. For example, in one embodiment, the invention includes a method of identifying dosage of a therapeutic agent that can inhibit PIP4K and/or can improve insulin signaling. Such a method can involve administering a series of test dosages of a therapeutic agent to an experimental animal that expresses PIP4K in somatic (e.g., muscle, fat, liver, nervous, and other) cells and observing which dosage(s) inhibits PIP4K, and/or improves insulin signaling in the experimental animal.

The present invention also provides a method of evaluating a therapeutically effective dosage for treating a diabetes with a PIP4K inhibitor or a test agent that includes determining the LD100 or ED50 of the agent in vitro. Such a method permits calculation of the approximate amount of agent needed per volume to improve insulin signaling. Such amounts can be determined, for example, by standard microdilution methods in cultured cells or by administration of varying amounts of a PIP4K inhibitor or a test agent to an experimental animal.

Test agents and test dosages that can successfully inhibit PIP4K activity, for example, by at least 2%, 5%, 10%, 20%, 40%, 50%, 60%, 70%, 80%, 90%, 95% or more than 95%. Test agents and test dosages that can successfully inhibit PIP4K activity to thereby improve insulin signaling, for example, by at least 2%, 5%, 10%, 20%, 40%, 50%, 60%, 70%, 80%, 90%, 95% or more than 95%. A therapeutically effective dosage is also one that is substantially non-toxic.

### Methods for Treatment of Cancer

Degradation or depletion of PIP4K2A, PIP4K2B, and/or PIP4K2C proteins is useful for preventing, treating and/or diagnosing cancer. In some cases, the PIP4K2C protein is targeted because it is expressed at higher levels in immune cells than the PIP4K2A and PIP4K2B proteins. Degradation or depletion of the PIP4K2A protein, the PIP4K2B protein, and/or particularly the PIP4K2C protein can enhance immune responses against cancer and tumors. Thus, one aspect of the invention is a method of treating or inhibiting the establishment and/or growth metastatic tumors in an animal (e.g., a human). Such a method involves administering compositions to the animal that degrade or deplete PIP4K2A, PIP4K2B, and/or especially PIP4K2C to thereby treat or inhibit the establishment and/or growth of cancer in an animal. Both human and veterinary uses are contemplated.

As illustrated herein PIP4Ks have a catalytic-independent role in regulating signaling within cells. Data provided herein shows that depletion of the three PIP4Ks in cells altered the levels of phosphoinositides beyond PI(5)P. Knockdown and knockout of PIP4Ks in cells, led to a surprising increase in PI(4,5)P₂ and concomitant decrease in PI(4)P. Basal levels of PI(3,4,5)P₃ during growth in culture are typically below the detection limit in HPLC-based assays. However, basal elevation of PI(3,4,5)P₃ was observed in cells with knockout PIP4K mutations, showing that the increased PI(4,5)P₂ is able to be utilized by PI3K. PI(4,5)P₂ constitutes ˜50% of all phosphoinositides, though the majority is bound to cytoskeletal and scaffolding proteins required for cell structure, migration, and adhesion. While PI(4,5)P₂ is highly abundant, local production of PI(4,5)P₂ by PIP5Ks offers spatiotemporal control of signaling through the PI3K and PLC pathways.

Methods are described herein for the treatment of cancer and to inhibit the progression of cancer. The methods of treating or inhibiting the progression of cancer and/or the establishment of metastatic tumors in an animal can include administering to a subject animal (e.g., a human), a therapeutically effective amount of a composition that degrades or depletes PIP4K2A protein, the PIP4K2B protein, and/or particularly the PIP4K2C protein. The methods of treating or inhibiting the establishment and/or growth metastatic tumors in an animal can also include administering such a composition with one or more other anti-cancer or chemotherapeutic agents.

In some embodiments, the methods can also include a detection step to ascertain whether the animal has cancer or is in need of treatment to inhibit the development of metastatic tumors. Such a detection step can include any available assay for cancer.

The term “animal” as used herein, refers to an animal, such as a warm-blooded animal, which is susceptible to or has a disease associated with protease expression, for example, cancer. Mammals include cattle, buffalo, sheep, goats, pigs, horses, dogs, cats, rats, rabbits, mice, and humans. Also included are other livestock, domesticated animals and captive animals. The term “farm animals” includes chickens, turkeys, fish, and other farmed animals. Mammals and other animals including birds may be treated by the methods and compositions described and claimed herein. In some embodiments, the animal is a human.

As used herein, the term “cancer” includes solid animal tumors as well as hematological malignancies. The terms “tumor cell(s)” and “cancer cell(s)” are used interchangeably herein.

“Solid animal tumors” include cancers of the head and neck, lung, mesothelioma, mediastinum, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin central nervous system; sarcomas of the soft tissue and bone; and melanoma of cutaneous and intraocular origin. In addition, a metastatic cancer at any stage of progression can be treated, such as micrometastatic tumors, megametastatic tumors, and recurrent cancers.

The term “hematological malignancies” includes childhood leukemia and lymphomas, Hodgkin's disease, lymphomas of lymphocytic and cutaneous origin, acute and chronic leukemia, plasma cell neoplasm and cancers associated with AIDS.

The inventive methods and compositions can also be used to treat cancer of the adrenal cortex, cancer of the cervix, cancer of the endometrium, cancer of the esophagus, cancer of the head and neck, cancer of the liver, cancer of the pancreas, cancer of the prostate, cancer of the thymus, carcinoid tumors, chronic lymphocytic leukemia, Ewing's sarcoma, gestational trophoblastic tumors, hepatoblastoma, multiple myeloma, non-small cell lung cancer, retinoblastoma, or tumors in the ovaries. A cancer at any stage of progression can be treated or detected, such as primary, metastatic, and recurrent cancers. Information regarding numerous types of cancer can be found, e.g., from the American Cancer Society (www.cancer.org), or from, e.g., Wilson et al. (1991) Harrison's Principles of Internal Medicine, 12th Edition, McGraw-Hill, Inc.

Treatment of, or treating, cancer can include the reduction in cancer cell growth, cancer cell migration, or the reduction in establishment of at least one metastatic tumor. The treatment also includes alleviation or diminishment of more than one symptom of cancer such as coughing, shortness of breath, hemoptysis, lymphadenopathy, enlarged liver, nausea, jaundice, bone pain, bone fractures, headaches, seizures, systemic pain and combinations thereof. The treatment may cure the cancer, e.g., it may prevent cancer, it may substantially eliminate tumor formation and growth, and/or it may arrest or inhibit the migration of metastatic cancer cells.

Anti-cancer activity can be evaluated against varieties of cancers using methods available to one of skill in the art. Anti-cancer activity, for example, can determined by identifying the lethal dose (LD100) or the 50% effective dose (ED50) or the dose that inhibits growth at 50% (GI50) of a composition or agent of the present invention. In one aspect, anti-cancer activity is the amount of the agent that reduces 50%, 60%, 70%, 80%, 90%, 95%, 97%, 98%, 99% or 100% of cancer cell growth or migration, for example, when measured by detecting the level of expression of a cancer cell marker or the expression of a cancer cell marker at sites distal from a primary tumor site, or when assessed using available methods for detecting metastases.

The compositions described herein for treatment of cancer can include additional therapeutic agents such as additional anti-cancer or chemotherapeutic agents, vitamins, pain reducing agents, and anti-microbial agents.

The anti-cancer agents useful in the compositions and methods described herein include cytotoxins, photosensitizing agents and chemotherapeutic agents. These agents include, but are not limited to, folate antagonists, pyrimidine antimetabolites, purine antimetabolites, 5-aminolevulinic acid, alkylating agents, platinum anti-tumor agents, anthracyclines, DNA intercalators, epipodophyllotoxins, DNA topoisomerases, microtubule-targeting agents, vinca alkaloids, taxanes, epothilones and asparaginases. Further information can be found in Bast et al., Cancer Medicine, edition 5, which is available free as a digital book (see website at ncbi.nlm.nih.gov/books/NBK20812/).

Folic acid antagonists are cytotoxic drugs used as antineoplastic, antimicrobial, anti-inflammatory, and immune-suppressive agents. While several folate antagonists have been developed, and several are now in clinical trial, methotrexate (MTX) is the antifolate with the most extensive history and widest spectrum of use. MTX is an essential drug in the chemotherapy regimens used to treat patients with acute lymphoblastic leukemia, lymphoma, osteosarcoma, breast cancer, choriocarcinoma, and head and neck cancer, as well as being an important agent in the therapy of patients with nonmalignant diseases, such as rheumatoid arthritis, psoriasis, and graft-versus-host disease.

Pyrimidine antimetabolites include fluorouracil, cytosine arabinoside, 5-azacytidine, and 2′. 2′-difluoro-2′-deoxycytidine. Purine antimetabolites include 6-mercatopurine, thioguanine, allopurinol (4-hydroxypyrazolo-3,4-d-pyrimidine), deoxycoformycin (pentostatin), 2-fluoroadenosine arabinoside (fludarabine; 9-β-d-arabinofuranosyl-2-fluoradenine), and 2-chlorodeoxyadenosine (Cl-dAdo, cladribine). In addition to purine and pyrimidine analogues, other agents have been developed that inhibit biosynthetic reactions leading to the ultimate nucleic acid precursors. These include phosphonacetyl-L-aspartic acid (PALA), brequinar, acivicin, and hydroxyurea.

Alkylating agents and the platinum anti-tumor compounds form strong chemical bonds with electron-rich atoms (nucleophiles), such as sulfur in proteins and nitrogen in DNA. Although these compounds react with many biologic molecules, the primary cytotoxic actions of both classes of agents appear to be the inhibition of DNA replication and cell division produced by their reactions with DNA. However, the chemical differences between these two classes of agents produce significant differences in their anti-tumor and toxic effects. The most frequently used alkylating agents are the nitrogen mustards. Although thousands of nitrogen mustards have been synthesized and tested, only five are commonly used in cancer therapy today. These are mechlorethamine (the original “nitrogen mustard”), cyclophosphamide, ifosfamide, melphalan, and chlorambucil. Closely related to the nitrogen mustards are the aziridines, which are represented in current therapy by thiotepa, mitomycin C. and diaziquone (AZQ). Thiotepa (triethylene thiophosphoramide) has been used in the treatment of carcinomas of the ovary and breast and for the intrathecal therapy of meningeal carcinomatosis. The alkyl alkane sulfonate, busulfan, was one of the earliest alkylating agents. This compound is one of the few currently used agents that clearly alkylate through an SN2 reaction. Hepsulfam, an alkyl sulfamate analogue of busulfan with a wider range of anti-tumor activity in preclinical studies, has been evaluated in clinical trials but thus far has demonstrated no superiority to busulfan.

Photosensitizing agents induce cytotoxic effects on cells and tissues. Upon exposure to light the photosensitizing compound may become toxic or may release toxic substances such as singlet oxygen or other oxidizing radicals that are damaging to cellular material or biomolecules, including the membranes of cells and cell structures, and such cellular or membrane damage can eventually kill the cells. A range of photosensitizing agents can be used, including psoralens, porphyrins, chlorines, aluminum phthalocyanine with 2 to 4 sulfonate groups on phenyl rings (e.g., AlPcS2a or AlPcS4), and phthalocyanins. Such drugs become toxic when exposed to light. For example, the photosensitizing agent can be an amino acid called 5-aminolevulinic acid, which is converted to protoporphyrin IX, a fluorescent photosensitizer. The structure of 5-aminolevulinic acid is shown below.

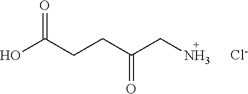

Topoisomerase poisons are believed to bind to DNA, the topoisomerase, or either molecule. Many topoisomerase poisons, such as the anthracyclines and actinomycin D, are relatively planar hydrophobic compounds that bind to DNA with high affinity by intercalation, which involves stacking of the compound between adjacent base pairs. Anthracyclines intercalate into double-stranded DNA and produce structural changes that interfere with DNA and RNA syntheses. Several of the clinically relevant anthracyclines are shown below.

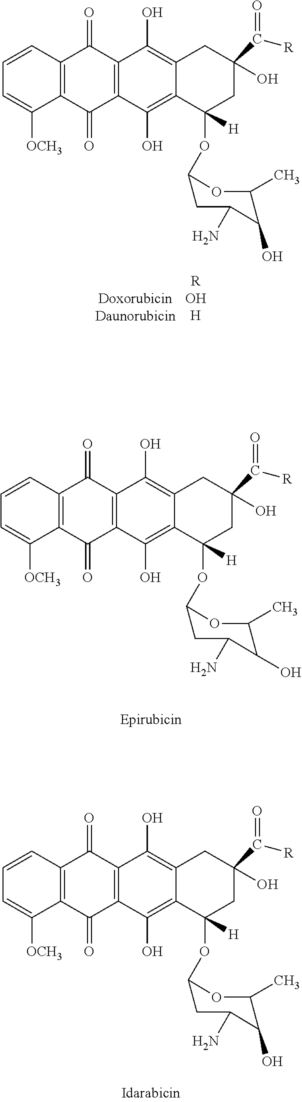

Non-intercalating topoisomerase-targeting drugs include epipodophyllotoxins such as etoposide and teniposide. Etoposide is approved in the United States for the treatment of testicular and small cell lung carcinomas. Etoposide phosphate is more water soluble than etoposide and is rapidly converted to etoposide in vivo. Other non-intercalating topoisomerase-targeting drugs include topotecan and irinotecan.

Unique classes of natural product anticancer drugs have been derived from plants. As distinct from those agents derived from bacterial and fungal sources, the plant products, represented by the Vinca and Colchicum alkaloids, as well as other plant-derived products such as paclitaxel (Taxol) and podophyllotoxin, do not target DNA. Rather, they either interact with intact microtubules, integral components of the cytoskeleton of the cell, or with their subunit molecules, the tubulins. Clinically useful plant products that target microtubules include the Vinca alkaloids, primarily vinblastine (VLB), vincristine (VCR), vinorelbine (Navelbine, VRLB), and a newer Vinca alkaloid, vinflunine (VFL; 20′,20′-difluoro-3′,4′-dihydrovinorelbine), as well as the two taxanes, paclitaxel and docetaxel (Taxotere). The structure of paclitaxel is provided below.

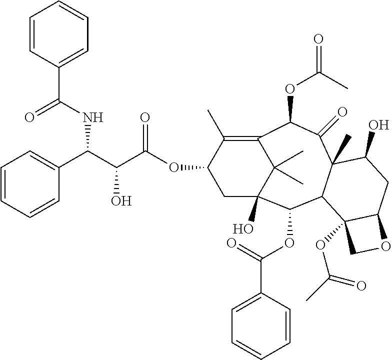 [SMILES: CC(=O)O[C@H]1C(=O)[C@@]2(C)C([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Preferably a paclitaxel moiety is linked to the peptide by C10 and/or C2 hydroxyl moiety.

Examples of drugs that can be used in the methods and compositions described herein include but are not limited to, aldesleukin, 5-aminolevulinic acid, asparaginase, bleomycin sulfate, camptothecin, carboplatin, carmustine, cisplatin, cladribine, cyclophosphamide (lyophilized), cyclophosphamide (non-lyophilized), cytarabine (lyophilized powder), dacarbazine, dactinomycin, daunorubicin, diethyistilbestrol, doxorubicin (doxorubicin, 4′-epidoxorubicin, 4- or 4′-deoxydoxorubicin), epoetin alfa, esperamycin, etidronate, etoposide, N,N-bis(2-chloroethyl)-hydroxyaniline, 4-hydroxycyclophosphamide, fenoterol, filgrastim, floxuridine, fludarabine phosphate, fluorocytidine, fluorouracil, fluorouridine, goserelin, granisetron hydrochloride, idarubicin, ifosfamide, interferon alpha-2a, interferon alpha-2b, leucovorin calcium, leuprolide, levamisole, mechiorethamine, medroxyprogesterone, melphalan, methotrexate, mitomycin, mitoxantrone, muscarine, octreotide, ondansetron hydrochloride, oxyphenbutazone, paclitaxel, pamidronate, pegaspargase, plicamycin, salicylic acid, salbutamol, sargramostim, streptozocin, taxol, terbutaline, terfenadine, thiotepa, teniposide, vinblastine, vindesine and vincristine. Other drugs that can be used in the methods and compositions described herein include those, for example, disclosed in WO 98/13059; Payne, 2003; US 2002/0147138 and other references available to one of skill in the art.

### Compositions

The PIP4K degrading agents, PIP4K inhibitors, PIP4K mutating agents, and/or PIP4K binding (e.g., antibody) agents can be formulated as compositions with or without additional therapeutic agents, and administered to an animal, such as a human patient, in a variety of forms adapted to the chosen route of administration. Routes for administration include, for example, oral, local, parenteral, intraperitoneal, intravenous and intraarterial routes.

The compositions can be formulated as pharmaceutical dosage forms. Such pharmaceutical dosage forms can include (a) liquid solutions; (b) tablets, sachets, or capsules containing liquids, solids, granules, or gelatin; (c) suspensions in an appropriate liquid; and (d) suitable emulsions.

Solutions of the active agents (PIP4K degrading agents, PIP4K antibodies, other therapeutic agents) can be prepared in water or saline, and optionally mixed with other agents. For example, formulations for intravenous or intraarterial administration may include sterile aqueous solutions that may also contain buffers, diluents, stabilizing agents, nontoxic surfactants, chelating agents, polymers and/or other suitable additives. Sterile injectable solutions are prepared by incorporating the active agents in the required amount in the appropriate solvent with various of the other ingredients, in a sterile manner or followed by sterilization (e.g., filter sterilization) after assembly.

In another embodiment, active agent-lipid particles can be prepared and incorporated into a broad range of lipid-containing dosage forms. For instance, the suspension containing the active agent-lipid particles can be formulated and administered as liposomes, gels, oils, emulsions, topical creams, pastes, ointments, lotions, foams, mousses, and the like.

In some embodiments, the active agents may be formulated in liposome compositions. Sterile aqueous solutions, active agent-lipid particles or dispersions comprising the active agent(s) are adapted for administration by encapsulation in liposomes. Such liposomal formulations can include an effective amount of the liposomally packaged active agent(s) suspended in diluents such as water, saline, or PEG 400.

The liposomes may be unilamellar or multilamellar and are formed of constituents selected from phosphatidylcholine, dipalmitoylphosphatidylcholine, cholesterol, phosphatidylethanolamine, phosphatidylserine, demyristoylphosphatidylcholine and combinations thereof. The multilamellar liposomes comprise multilamellar vesicles of similar composition to unilamellar vesicles but are prepared to provide a plurality of compartments in which the silver component in solution or emulsion is entrapped. Additionally, other adjuvants and modifiers may be included in the liposomal formulation such as polyethyleneglycol, or other materials.

While a suitable formulation of liposome includes dipalmitoyl-phosphatidylcholine:cholesterol (1:1) it is understood by those skilled in the art that any number of liposome bilayer compositions can be used in the composition of the present invention. Liposomes may be prepared by a variety of known methods such as those disclosed in U.S. Pat. No. 4,235,871 and in RRC, Liposomes: A Practical Approach. IRL Press, Oxford, 1990, pages 33-101.

The liposomes containing the active agents may have modifications such as having non-polymer molecules bound to the exterior of the liposome such as haptens, enzymes, antibodies or antibody fragments, cytokines and hormones and other small proteins, polypeptides or non-protein molecules which confer a desired enzymatic or surface recognition feature to the liposome. Surface molecules which preferentially target the liposome to specific organs or cell types include for example antibodies which target the liposomes to cells bearing specific antigens. Techniques for coupling such molecules are available (see for example U.S. Pat. No. 4,762,915 the disclosure of which is incorporated herein by reference). Alternatively, or in conjunction, one skilled in the art would understand that any number of lipids bearing a positive or negative net charge may be used to alter the surface charge or surface charge density of the liposome membrane. The liposomes can also incorporate thermal sensitive or pH sensitive lipids as a component of the lipid bilayer to provide controlled degradation of the lipid vesicle membrane.

Liposome formulations for use with active agents may also be formulated as disclosed in WO 2005/105152 (the disclosure of which is incorporated herein in its entirety). Briefly, such formulations comprise phospholipids and steroids as the lipid component. These formulations help to target the molecules associated therewith to in vivo locations without the use of an antibody or other molecule.

Antibody-conjugated liposomes, termed immunoliposomes, can be used to carry active agent(s) within their aqueous compartments. Compositions of active agent(s) provided within antibody labeled liposomes (immunoliposomes) can specifically target the active agent(s) to a particular cell or tissue type to elicit a localized effect. Methods for making of such immunoliposomal compositions are available, for example, in Selvam M. P., et al., 1996. Antiviral Res. Dec; 33(1):11-20 (the disclosure of which is incorporated herein in its entirety).

For example, immunoliposomes can specifically deliver active agents to the cells possessing a unique antigenic marker recognized by the antibody portion of the immunoliposome. Immunoliposomes are ideal for the in vivo delivery of active agent(s) to target tissues due to simplicity of manufacture and cell-specific specificity.

Muscle cell-specific antibodies, fat-cell specific antibodies, liver-cell specific antibodies, and other somatic cell-specific types of antibodies can be used in conjunction with the inhibitors or liposomes containing inhibitors to help target the inhibitors and liposomes to specific cell types. Other active agents can also be included in such liposomes.

In some instances, the active agents can be administered orally, in combination with a pharmaceutically acceptable vehicle such as an inert diluent or an assimilable edible carrier. They may be enclosed in hard or softshell gelatin capsules, may be compressed into tablets, or may be incorporated directly with the food of the patient's diet. For oral therapeutic administration, they may be combined with one or more excipients and used in the form of ingestible tablets, buccal tablets, troches, capsules, elixirs, suspensions, syrups, wafers, and the like. Such compositions and preparations may contain at least 0.1% of active compound. The percentage of the compositions and preparations may, of course, be varied. The amount of compound in such therapeutically useful compositions is such that an effective dosage level will be obtained.

The active agents can also be incorporated into dosage forms such as tablets, troches, pills, and capsules. These dosage forms may also contain any of the following: binders such as gum tragacanth, acacia, corn starch or gelatin; excipients such as dicalcium phosphate; a disintegrating agent such as corn starch, potato starch, alginic acid and the like; a lubricant such as magnesium stearate; polymers such as cellulose-containing polymers (e.g., hydroxypropyl methylcellulose, methylcellulose, ethylcellulose), polyethylene glycol, poly-glutamic acid, poly-aspartic acid or poly-lysine; and a sweetening agent such as lactose or aspartame or a flavoring agent such as peppermint, oil of wintergreen, or cherry flavoring may be added.

Tablet formulations can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active agents in a flavoring or sweetener, e.g., as well as pastilles comprising the active agent(s) in an inert base, such as gelatin and glycerin or sucrose and acacia emulsions, gels, and the like containing carriers available in the art.

When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form should be pharmaceutically acceptable and substantially non-toxic in the amounts employed. In addition, the active compounds and agents may be incorporated into sustained-release preparations and devices.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

In some embodiments, one or more of the active agents are linked to polyethylene glycol (PEG). For example, one of skill in the art may choose to link an active agent to PEG to form the following pegylated drug.

Useful dosages of the active agents (e.g., PIP4K degrading agent) can be determined by comparing their in vitro activity, and in vivo activity in animal models, for example, as described herein. Methods for the extrapolation of effective dosages in mice, and other animals, to humans are available to the art; for example, see U.S. Pat. No. 4,938,949. The agents can be conveniently administered in unit dosage form.

The desired dose may conveniently be presented in a single dose or as divided doses administered at appropriate intervals, for example, as two, three, four or more sub-doses per day. The sub-dose itself may be further divided, for example, into a number of discrete loosely spaced administrations; such as multiple oral, intraperitoneal or intravenous doses. For example, it can be desirable to administer the present compositions intravenously over an extended period, either by continuous infusion or in separate doses.

The therapeutically effective amount of the active agent(s) (e.g., PIP4K degrading agents) necessarily varies with the subject and the disease, disease severity, or physiological problem to be treated. As one skilled in the art would recognize, the amount can be varied depending on the method of administration. The amount of the active agent (e.g., inhibitor) for use in treatment will vary not only with the route of administration, but also the nature of the condition being treated and the age and condition of the patient and will be ultimately at the discretion of the attendant physician or clinician.

The pharmaceutical compositions of the invention can include an effective amount of at least one of the active agents of the invention (e.g., PIP4K degrading agents), or two or more different agents of the invention (e.g., two or more PIP4K inhibitors or degrading agents). These compositions can also include a pharmaceutically effective carrier.

The pharmaceutical compositions of the invention can also include other active ingredients and therapeutic agents, for example, anti-diabetes agents, anti-inflammatory agents, analgesics, vitamins, and the like. It is also within the scope of the present invention to combine any of the methods and any of the compositions disclosed herein with conventional diabetes therapies and various drugs in order to enhance the efficacy of such methods and/or compositions. For example, methods and compositions containing combinations of active agents can act through different mechanisms to improve the efficacy or speed of treatment. Methods and compositions containing combinations of active agents can also reduce the doses/toxicity of conventional therapies and/or to increase the sensitivity of conventional therapies.

For example, a variety of pharmaceutical preparations of insulin or diabetes medications can be used in combination with the methods and compositions described herein. For example, any of the following can be used with the methods and compositions described herein in the treatment of diabetes, such as regular insulin (such as Actrapid®), isophane insulin (designated NPH), insulin zinc suspensions (such as Semilente®, Lente®, and Ultralente®), and biphasic isophane insulin (such as NovoMix®). Human insulin analogues and derivatives have also been developed, designed for particular profiles of action, i.e. fast action or prolonged action. The long-acting insulin analogue, degludec (Begin™), as well as a biphasic preparation of degludec and the fast-acting insulin aspart, DegludecPlus (BOOST™), may be used. Some of the commercially available insulin preparations comprising rapid acting insulin analogues include NovoRapid® (preparation of B28Asp human insulin), Humalog® (preparation of B28LysB29Pro human insulin) and Apidra® (preparation of B3LysB29Glu human insulin). Some of the commercially available insulin preparations comprising long-acting insulin analogues include Lantus® (preparation of insulin glargine) and Levemir® (preparation of insulin detemir).

Monoclonal antibodies, nucleic acid inhibitors, and gene therapy are targeted therapies that can also be combined into the PIP4K inhibitor compositions and used in the methods described herein.

The ultimate dosage form should be sterile, fluid and stable under the conditions of manufacture and storage.

### Kits

Another aspect of the invention is one or more kits for inhibiting PIP4K or treating insulin resistance and diabetes.

The kits of the present invention can include PIP4K inhibitor, reagents for modifying genomic PIP4K sites, or other therapeutic reagents, or a combination thereof. The kits can also include instructions for administering the PIP4K inhibitor, reagents for modifying genomic PIP4K sites, or other therapeutic reagents.

In some cases, the kit can include reagents for isolating cells (e.g. muscle, liver, fat, nervous, and/or other types of cells) from a subject and modifying genomic PIP4K sites. Such kits can include sterile implements for isolating cells from a subject, reagents for culturing cells, one or more guide RNA(s) for targeting genomic PIP4K sites, implements for administering modified cells back into the subject, and any combination thereof.

The following non-limiting Examples illustrate materials and methods used for development of the invention. Appendix A may provide further information.

### Example 1: Materials and Methods

This Example illustrates some of the materials and methods employed in the development of the invention.

### Cell Lines, Authentication:

Cell lines were purchased from ATCC and/or fingerprinted with the University of Arizona genetics core. Cells were tested to be mycoplasma free with Lonza Mycoalert.

### Cell Culture Conditions:

293T, HeLa, PaTu 8988t, and BJ cells were cultured using DMEM media supplemented with 10% FBS, glutamine and pyruvate. H1299 and H1975 cells were cultured in RPMI media.

### Cell Lysis and Immunoblotting:

Cells were lysed in RIPA buffer supplemented with 1 tablet of protease and phosphatase inhibitor. After incubation on ice for 20 minutes, lysates were cleared by centrifugation at 14,000×g and supernatant was quantified using BCA assay. Lysates were subjected to SDS-PAGE using Novex NuPAGE system. Proteins were separated on 4-12% Bis Tris Pre-Cast Gels 10% Bis Tris gels using MOPS buffer. Proteins were transferred to 0.45 μm nitrocellulose membranes at 350 mA for 1 h. Membranes were blocked in 5% non-fat milk in TBST and incubated with primary antibody overnight: For chemiluminescently detect antibody binding, membranes were blotted with HRP conjugated secondary antibodies. Membranes were developed using ECL solution and exposed to film. For insulin signaling westerns, protocol was modified such that cells were lysed in triton buffer, IRDye secondary was used for LiCor Odyssey detection with quantification using Image Studio Lite software.

### Generation of Lentivirus, Viral Transduction:

293T cells were used to generate lentivirus. Once cells were at 90-95% confluence in a 10 cm dish, transfection was performed with Opti-mem, Lipofectamine 2000, lentiviral vector, and accessory plasmids VSVG and Δ8.2. Virus supernatant was harvested 2 days and 3 days post transfection, then filtered and concentrated in an uhracentrifuge at 25,000 rpm for 120 minutes at 4° C.

Generation of Cell Lines with CRISPR Knockout of PIP4K:

CRISPR guides in pX458 were transfected in 293T cells. At 48-96 hours post transfection, GFP positive cells were single-cell sorted in 96-well plates using the Influx sorter at the WCMC Flow Cytometry Core. Two weeks later, wells were scored to contain single cell colony and expanded to screen for successful PIP4K2A/PIP4K2B/PIP4K2C knockout. Validation was performed by western blotting as well as PCR around each cut site.

Generation of Cell Lines with miRE Knockdown of PINK

LT3GEPIR vectors containing desired miR-E shRNA(s) were double digested downstream of existing shRNA(s) with EcoRI-HF/MluI-HF and PCR purified. The miR-E sequence to be added was PCR amplified with Multi-sh-F and Multi-sh-R. PCR products were purified, double digested with BbsI/MluI, and PCR purified once more. Ligations were performed with PCR product and open LT3GEPIR vectors using T4 ligase. Colonies were screened using miRE-F. The primers employed are listed in Table 1.

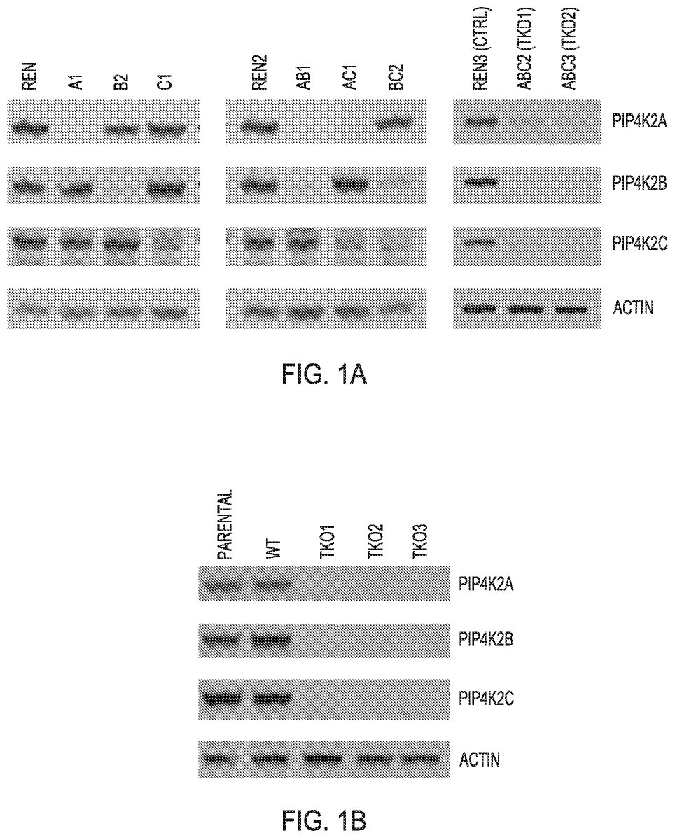

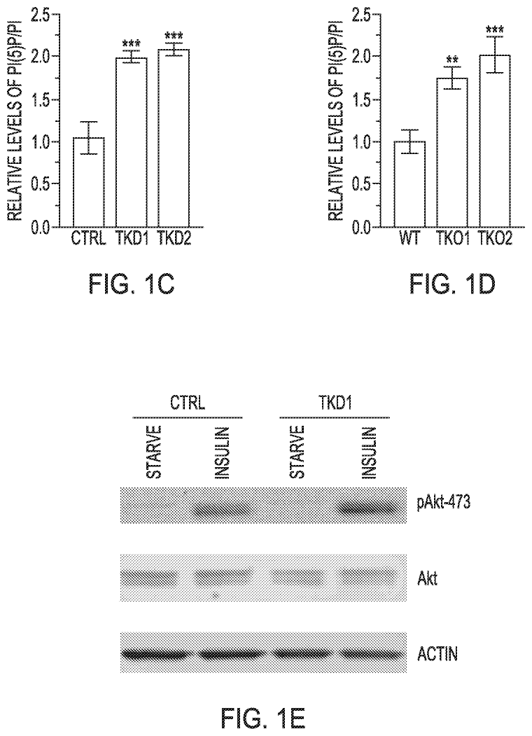

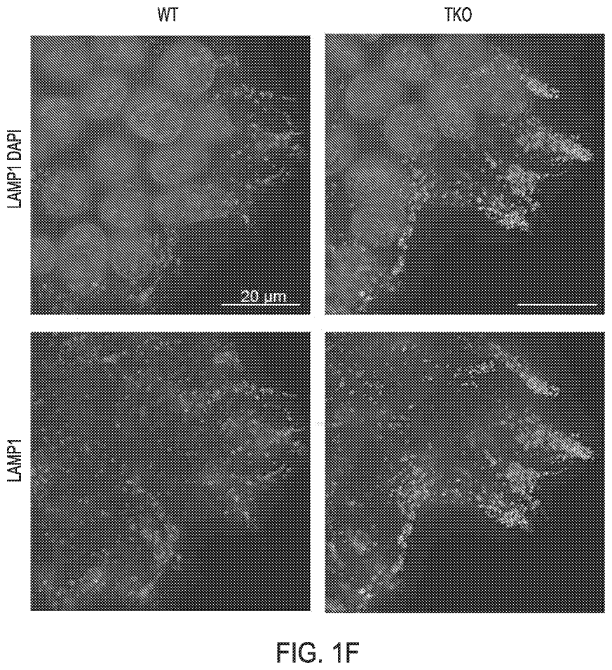

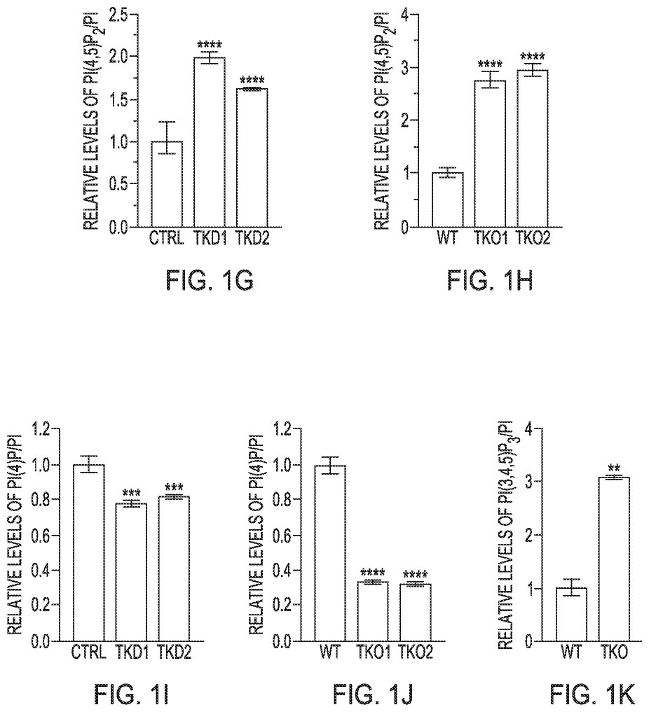

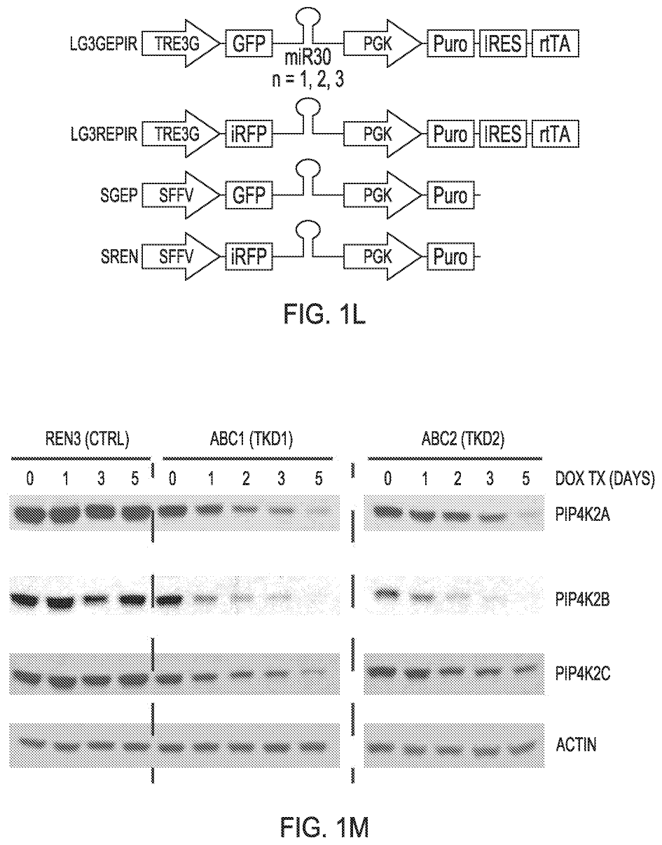

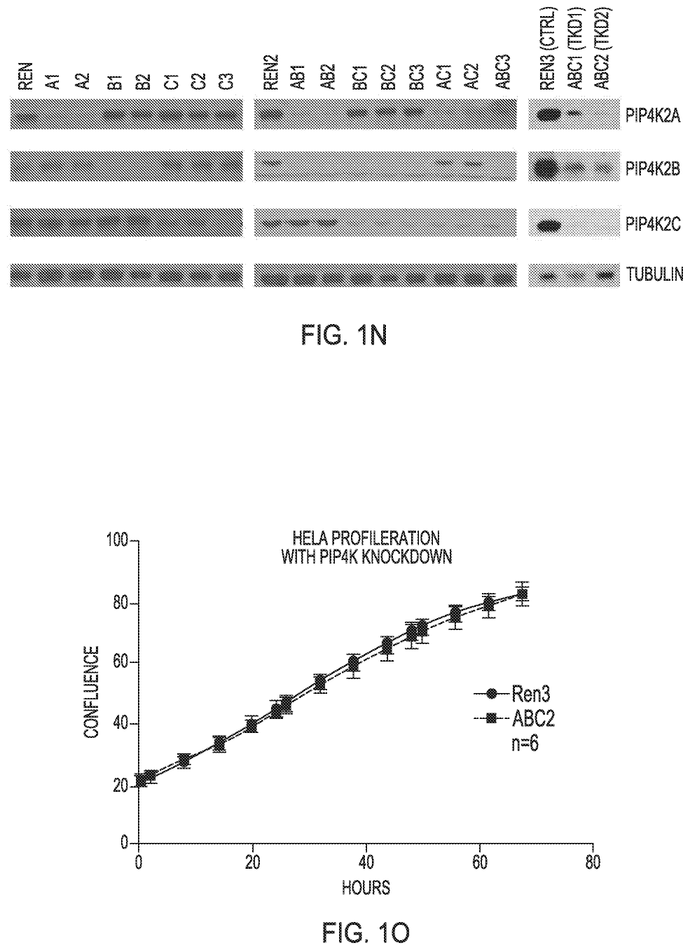

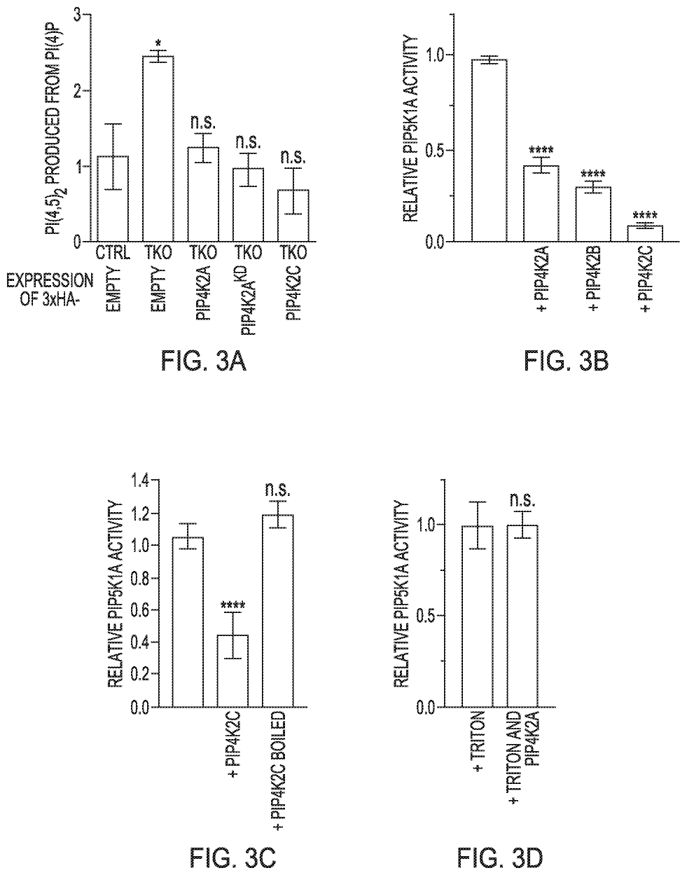

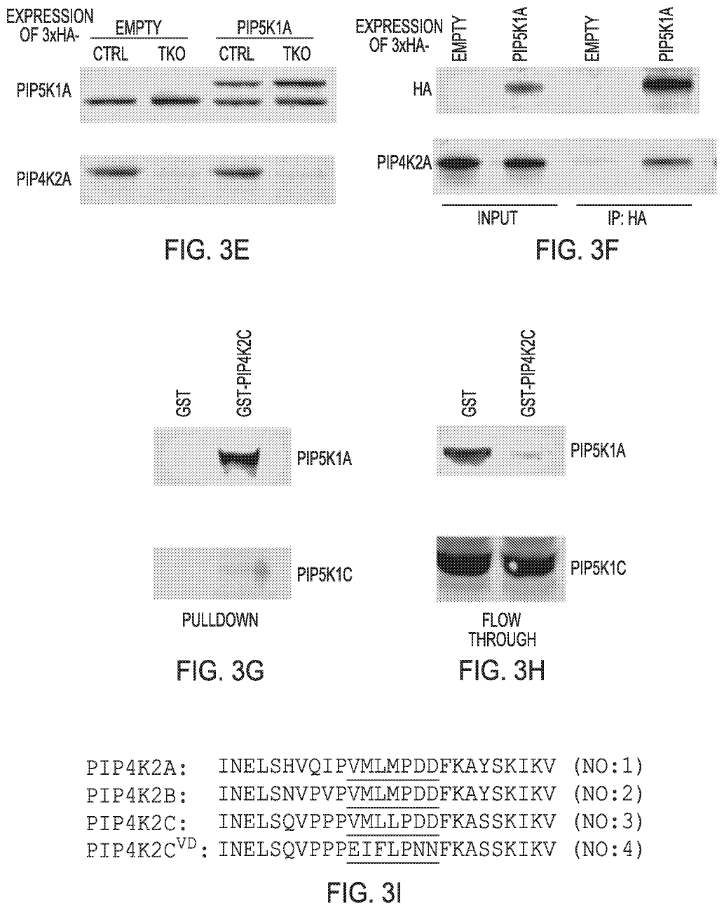

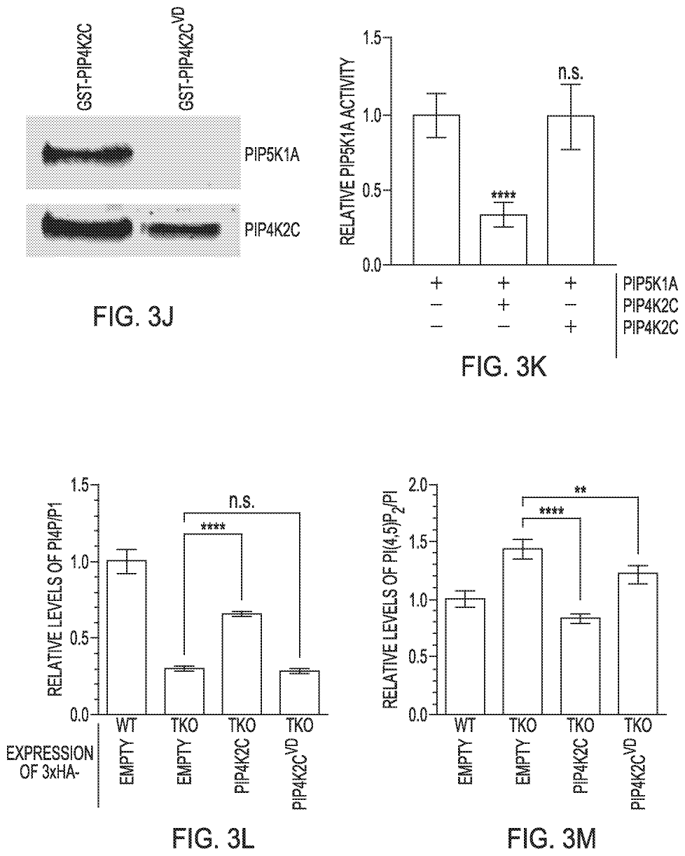

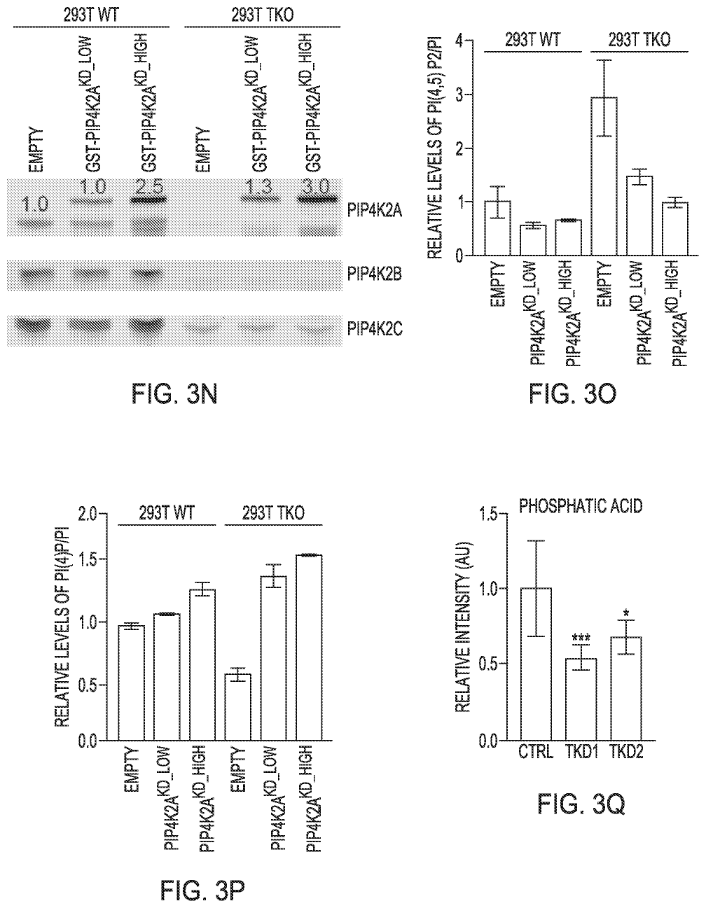

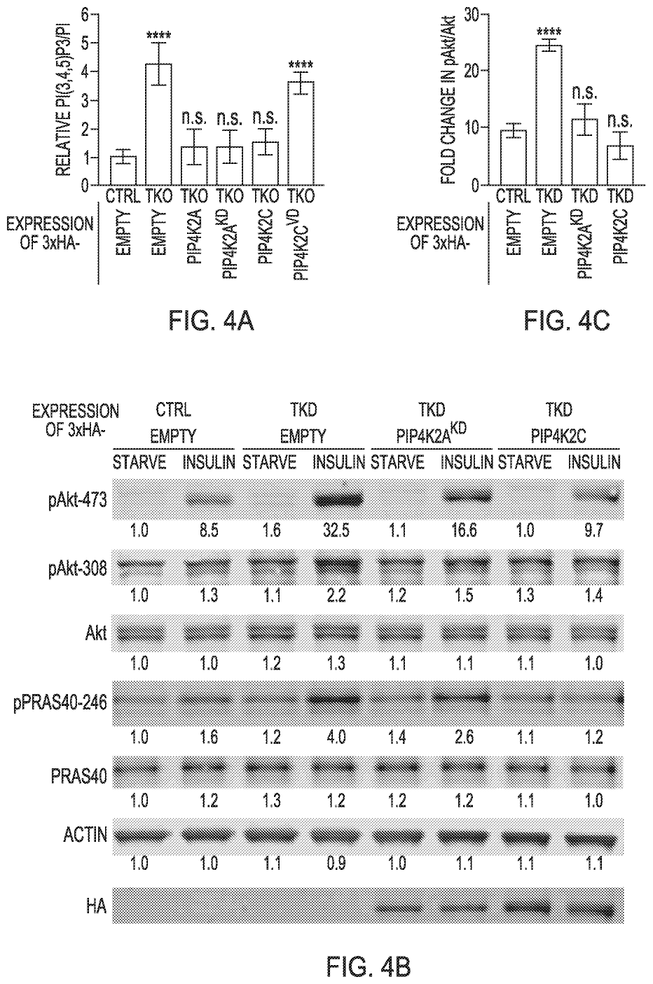

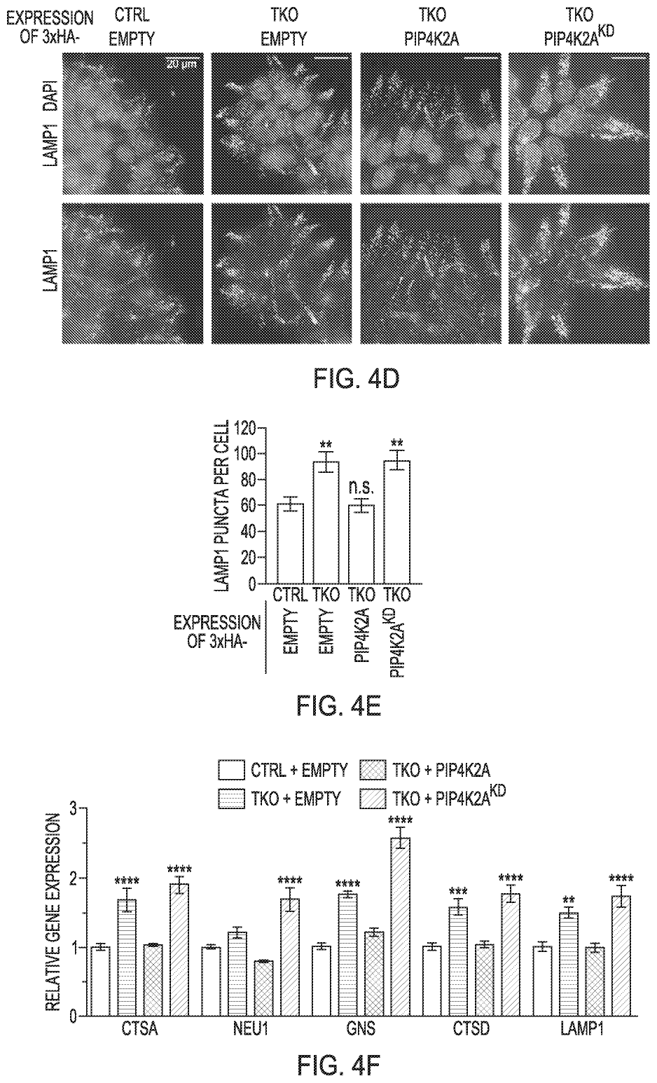

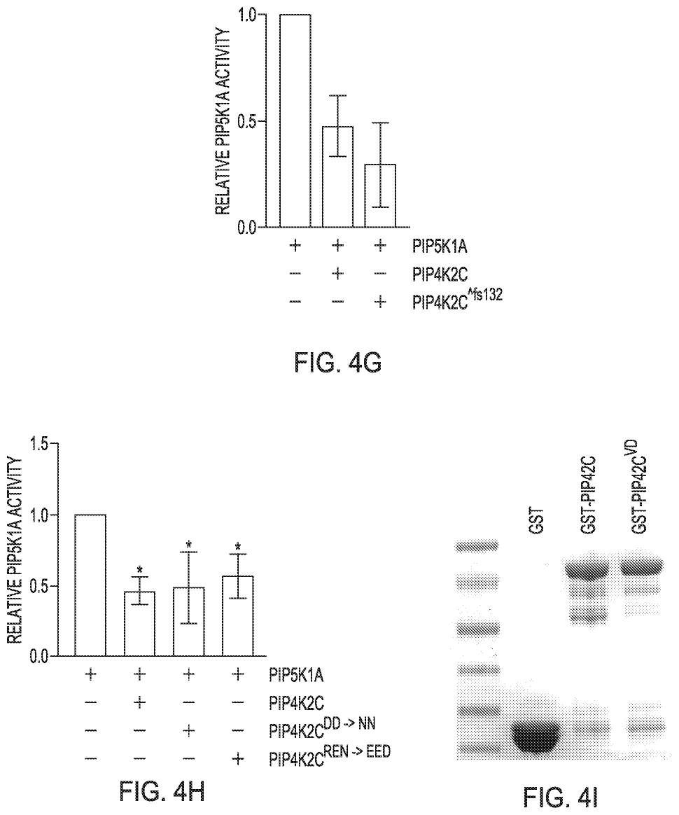

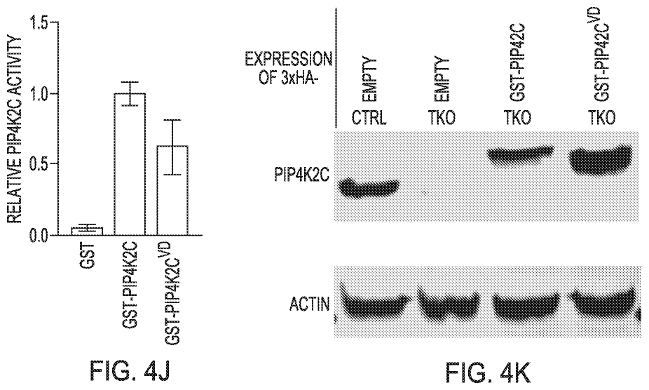

TABLE 1
Nucleic Acids, Primers, etc.

| | | Figure/ |
|---|---|---|
| Oligo | Sequence | Experiment |
| miRE-F | TGTTTGAATGAGGCTTCAGTAC | FIG. 1A/ |
| | (SEQ ID NO: 27) | hairpin |
| Multi-sh-F | AGGCGCGAAGACTCAATTGAAGGCTC | FIG. 1A/ |
| | GAGAAGGTATATTGCTG | hairpin |
| | (SEQ ID NO: 28) | |
| Multi-sh-R | CACTTTTTTCAATTGACACGTACGCGT | FIG. 1A/ |
| | ATTCTACCGGGTA | hairpin |
| | (SEQ ID NO: 29) | |
| CRISPR_2A- | AGAGTGGATGGGCAAGAAGC | FIG. 1B/ |
| F outside | (SEQ ID NO: 30) | CRISPR |
| CRISPR-2A- | AAGATGGAGTCATTGCTGTTCA | FIG. 1B/ |
| R outside | (SEQ ID NO: 31) | CRISPR |
| CRISPR-2A-F | GATTGACTCTCCCTCACCACT | FIG. 1B/ |
| | (SEQ ID NO: 32) | CRISPR |
| CRISPR-2A-R | CTGTGTACAAGAGCAGAGGTTC | FIG. 1B/ |
| | (SEQ ID NO: 33) | CRISPR |
| CRISPR_2B- | TGCTTGAGCTCAGGACAGTG | FIG. 1B/ |
| F outside | (SEQ ID NO: 34) | CRISPR |
| CRISPR-2B- | ACTAAGACCAAGATGGGGCC | FIG. 1B/ |
| R outside | (SEQ ID NO: 35) | CRISPR |
| CRISPR-2B-F | GCTGGTGTGGGCAGATTGCT | FIG. 1B/ |
| | (SEQ ID NO: 36) | CRISPR |
| CRISPR-2B-R | CACTGCTACAGCCTCACACTG | FIG. 1B/ |
| | (SEQ ID NO: 37) | CRISPR |
| CRISPR_2C- | GATTGCCTGCATTCGCTCTG | FIG. 1B/ |
| F outside | (SEQ ID NO: 38) | CRISPR |
| CRISPR-2C- | ATGCTGCTGTTTGGATGGGT | FIG. 1B/ |
| R outside | (SEQ ID NO: 39) | CRISPR |
| CRISPR-2C-F | GTTCTCATGGCATCTCCAAGG | FIG. 1B/ |
| | (SEQ ID NO: 40) | CRISPR |
| CRISPR-2C-R | GACTGTTGTGAGCATGAAGTTC | FIG. 1B/ |
| | (SEQ ID NO: 41) | CRISPR |
| U6-F | GAGGGCCTATTTCCCATGATT | FIG. 1B/ |
| | (SEQ ID NO: 42) | CRISPR |
| Quik- | CCAGCTGAAGCTCATGAACTACAGTC | FIG. 2/ |
| 2AD273N-F | TGCTGGTGGGAATTCAT | transgene |
| | (SEQ ID NO: 43) | |
| Quik- | ATGAATTCCCACCAGCAGACTGTAGT | FIG. 2/ |
| 2AD273N-R. | TCATGAGCTTCAGCTGG | transgene |
| | (SEQ ID NO: 44) | |
| Quik- | TAGGAAGGAGGTGTACTTCATGGCAA | FIG. 2/ |
| 2AD359N-F | TTATTACATCCTTACTCATT | transgene |
| | (SEQ ID NO: 45) | |
| Quik- | AATGAGTAAGGATGTTAATAATTGCCA | FIG. 2/ |
| 2AD359N-R | TGAAGTACACCTCCTTCCTA | transgene |
| | (SEQ ID NO: 46) | |
| Quik- | ACAGCTGAAGATCATGAACTACAGCC | FIG. 2/ |
| 2BD278N-F | TGCTGGTGGGCATCCAC | transgene |
| | (SEQ ID NO: 47) | |
| Quik- | GTGGATGCCCACCAGCAGGCTGTAGT | FIG. 2/ |
| 2BD278N-R | TCATGATCTTCAGCTGT | transgene |
| | (SEQ ID NO: 48) | |
| Quik- | GTATTTCATGGCCATCATTAATATCCT | FIG. 2/ |
| 2BD2369N-F | CACGCCATACGATA | transgene |
| | (SEQ ID NO: 49) | |
| Quik- | TATCGTATGGCGTGAGGATATTAATGA | FIG. 2/ |
| 2BD369N-R | TGGCCATGAAATAC | transgene |
| | (SEQ ID NO: 50) | |
| Quik- | AGTGCAGCTGAAGATCATGAACTACA | FIG. 2/ |
| 2CD280N-F | GCCTTCTGCTAGGCATCC | transgene |
| | (SEQ ID NO: 51) | |
| Quik- | GGATGCCTAGCAGAAGGCTGTAGTTC | FIG. 2/ |
| 2CD280N-R | ATGATCTTCAGCTGCACT | transgene |
| | (SEQ ID NO: 52) | |
| Quik- | CCAGAAGGAGGTCTACTTCATGGGCC | FIG. 2/ |
| 2CD374N-F | TCATTGATATCCTTACACAG | transgene |
| | (SEQ ID NO: 53) | |
| Quik- | CTGTGTAAGGATATCAATGAGGCCCA | FIG. 2/ |
| 2CD374N-R | TGAAGTAGACCTCCTTCTGG | transgene |
| | (SEQ ID NO: 54) | |
| Quik-2A650665_ | GCATGTACCGGCTTAATGTTGATGGT | FIG. 2/ |
| wobble-F | GTAGAGATTTACGTCATTGTAACTAGG | transgene |
| | AACGTATTCAGCCACCGTTTGTCTGT | |
| | GTATAGG (SEQ ID NO: 55) | |
| Quik-2A650665_ | CCTATACACAGACAAACGGTGGCTGA | FIG. 2/ |
| wobble-R | ATACGTTCCTAGTTACAATGACGTAAA | transgene |
| | TCTCTACACCATCAACATTAAGCCGGT | |
| | ACATGC (SEQ ID NO: 56) | |
| Quik-2A948- | CTTCCGTAACCTGCGGGAAAGATTCG | FIG. 2/ |
| wobble-F | GTATAGACGACCAAGATTTCCAGAATT | transgene |
| | CCCTG (SEQ ID NO: 57) | |
| Quick- | CAGGGAATTCTGGAAATCTTGGTCGT | FIG. 2/ |
| 2A948- | CTATACCGAATCTTTCCCGCAGGTTAC | transgene |
| wobble-R | GGAAG (SEQ ID NO: 58) | |
| Quik- | CATTGATATCCTCACCCCTTATGACAC | FIG. 2/ |
| 2B130_ | TAAAAAAAAAGCTGCACATGCTGCCA | transgene |
| wobble-F | AAAC (SEQ ID NO: 59) | |
| Quik- | GTTTTGGCAGCATGTGCAGCTTTTTTT | FIG. 2/ |
| 2B130_ | TTAGTGTCATAAGGGGTGAGGATATC | transgene |
| wobble-R | AATG (SEQ ID NO: 60) | |
| Quik- | CCAGCGAGGACGTGGCGGAGATGCA | FIG. 2/ |
| 2B868_ | TAATATATTGAAAAAGTACCACCAGTT | transgene |
| wobble-F | TATAG (SEQ ID NO: 61) | |
| Quik- | CTATAAACTGGTGGTACTTTTTCAATA | FIG. 2/ |
| 2B868_ | TATTATGCATCTCCGCCACGTCCTCG | transgene |
| wobble-R | CTGG (SEQ ID NO: 62) | |
| Quik-2CDD_NNf | GGTTATGCTGCTGCCGAACAATTTCA | FIG. 3 |
| | AGGCGAGCAG (SEQ ID NO: 63) | |
| Quik-2CDD_NNr | CTGCTCGCCTTGAAATTGTTCGGCAG | FIG. 3 |
| | CAGCATAACC (SEQ ID NO: 64) | |
| Quik- | ATTTGAAGTGGCTCGGCAGGTCTTCC | FIG. 3 |
| 2CREN_EEDf | TCGTGAAACAGGTGGTTGTTAACC | |
| | (SEQ ID NO: 65) | |
| Quik- | GGTTAACAACCACCTGTTTCACGAGG | FIG. 3 |
| 2CREN_EEDr | AAGACCTGCCGAGCCACTTCAAAT | |
| | (SEQ ID NO: 66) | |
| Quik-2CVDf | AGCCAGGTGCCGCCGCCGGAGATAT | FIG. 3 |
| | TCCTGCCGAACAATTTCAAGG | |
| | (SEQ ID NO: 67) | |
| Quik-2CVDr | CCTTGAAATTGTTCGGCAGGAATATCT | FIG. 3 |
| | CCGGCGGCGGCACCTGGCT | |
| | (SEQ ID NO: 68) | |
| Neu1-F | ACCTTGGGGCAGTAGTGAG | FIG. 4F/ |
| | (SEQ ID NO: 69) | RT-PCR |
| Neu1-R | TCCCGCTGTTTCTGAATACCA | FIG. 4F/ |
| | (SEQ ID NO: 70) | RT-PCR |
| GNS-F | GCATGACACCGCTAAAGAAAAC | FIG. 4F/ |
| | (SEQ ID NO: 71) | RT-PCR |
| GNS-R | CACAACGTGATGATTATGTGGGT | FIG. 4F/ |
| | (SEQ ID NO: 72) | RT-PCR |
| CTSD-F | ATTCAGGGCGAGTACATGATCC | FIG. 4F/ |
| | (SEQ ID NO: 73) | RT-PCR |
| CTSD-R | CGACACCTTGAGCGTGTAG | FIG. 4F/ |
| | (SEQ ID NO: 74) | RT-PCR |
| Lamp1-F | CACGAGAAATGCAACACGTTAC | FIG. 4F/ |
| | (SEQ ID NO: 75) | RT-PCR |
| Lamp1-R | GGGTGCCACTAACACATCTGTAT | FIG. 4F/ |
| | (SEQ ID NO: 76) | RT-PCR |
| Beta-Actin-F | ATAGCACAGCCTGGATAGCAACGTAC | FIG. 4F/ |
| | (SEQ ID NO: 77) | RT-PCR |
| Beta-Actin-R | CACCTTCTACAATGAGCTGCGTGTG | FIG. 4F/ |
| | (SEQ ID NO: 78) | RT-PCR |

### Generation of Mammalian and Bacterial Expression Vectors:

To generate vectors to express hairpin-resistant coding sequences for PIP41K isoforms, we cloned wild type PIP4K2A and PIP4K2C into a lentiviral backbone with a PGK promoter. Next, we generated mutations in PIP41K cDNA using Quik-Change to make kinase-dead variants and silent mutations in wobble positions for hairpin-resistant cDNA. Primers employed are listed in Table 1. No wobble mutations were needed for PIP4K2C cDNA since all hairpins targeted the 3′ UTR. To generate PIP41K2C N-terminal mutant %, Quikchange kit from Agilent (220521) was used and listed in Table 1.

Measurement of Phosphoinositides with High Performance Liquid Chromatography:

Cellular phosphoinositides were metabolically labeled for 48 hours in inositol-free DMEM supplemented with glutamine, 10% dialyzed FBS, and 10 μCi/mL 3H myo-inositol. Cells were washed with PBS and then transferred on ice. Cells were killed and then harvested by scraping using 1.5 mL ice-cold aqueous solution (1M HCl, 5 mM Tetrabutylammonium bisulfate, 25 mM EDTA). 2 mL of ice cold MeOH and 4 mL of CHCl₃ were added to each sample. After ensuring each vial is tightly capped, samples were vortexed and then centrifuged at 1000 rpm for 5 min. If a significant intermediate layer was visible, sample were gently agitated and spun again, until there were predominately two clear layers. The organic layer (lower) was cleaned using theoretical upper, while the aqueous layer was cleaned using theoretical lower (theoretical upper and lower made by combining CHCl3:MeOH:aqueous solution in 8:4:3 v/v ratio). Organic phases were collected and dried under nitrogen gas. Lipids were deacylated using monomethylamine solution (47% Methanol, 36% of 40% Methylamine, 9% butanol, and 8% H₂O, by volume). Samples were incubated at 550 for 1 hour and subsequently dried under nitrogen gas. To the dried vials, 1 mL of theoretical upper and 1.5 mL of theoretical lower were added (theoretical upper and lower made by combining CHCl₃:MeOH:H₂O in 8:4:3 v/v ratio). Samples were vortexed and spun at 1000 rpm. The aqueous phase (upper) was collected and dried under nitrogen gas. Samples were resuspended in 150 μL Buffer A (1 mM EDTA), filtered and transferred to Agilent polypropylene tubes. Samples were analyzed by anion-exchange HPLC using Partisphere SAX column. The compounds were eluted with a gradient starting at 100% Buffer A (1 mM EDTA) and increasing Buffer B (1 mM EDTA, 1M NaH₂PO₄) over time: 0-1 min 100% Buffer A, 1-30 min 98% Buffer A/2% Buffer B, 30-31 min 86% Buffer A/14% Buffer B, 31-60 min 70% Buffer A/30% Buffer B, 60-80 min 34% Buffer A/66% Buffer B, 80-85 min 100% Buffer B, 85-120 min 100% Buffer A. Buffers were pumped at 1 mL/min through column. Eluate from the HPLC column flowed into an on-line continuous flow scintillation detector for isotope detection. The detector was set to observe events between 10 minutes and 85 minutes, with scintillation fluid flowing at 4 mL/min.

### Measurement of Phosphatidic Acid Using LCMS:

Cellular lipids were extracted using same method described for phosphoinositide analysis. Lipids were dried and diluted with 113 μL of chloroform:methanol:water (73:23:3) mixture and filtered (0.45 μm) before analysis on an Agilent 6230 electrospray ionization-time-of-flight (ESI-TOF) MS coupled to an Agilent 1260 HPLC equipped with a Phenomenex Luna silica 3 μm 100 Å 5 cm×2.0 mm column. LCMS analysis was performed using normal phase HPLC with a binary gradient elution system where solvent A was chloroform:methanol:ammonium hydroxide (85:15:0.5) and solvent B was chloroform:methanol:water:ammonium hydroxide (60:34:5:0.5). Separation was achieved using a linear gradient from 100% A to 100% B over 9 min. Phospholipid species were detected using a dual ESI source operating in positive mode, acquiring in extended dynamic range from m/z 100-1700 at one spectrum per second; gas temperature: 325° C.; drying gas 10 L/min; nebulizer: 20 psig; fragmentor 300 V.

### Immunoprecipitation:

293T cell lines expressing 3×HA empty vector or 3×HA-PIP5K1A were fixed for 10 minutes in 4% PFA, washed 3× with PBS, and lysed in lysis buffer (10 mM Tris 7.4, 150 mM NaCl, 0.5 mM EDTA, 0.5% NP40) using probe sonicator for 2 minutes. Lysates were pre-cleared with control magnetic beads for 1 hour and 4° and Pierce magnetic anti-HA beads added for overnight incubation at 4°. Beads were washed 3× with lysis buffer and aliquots were added to sample buffer loading dye with beta mercaptoethanol, boiled, and run on SDS-page for immunoblotting.

### Protein Purification:

BL21 bacterial cells were transfected with pGEX vectors with PIP4K isoform coding sequence variants. Cells were grown in 1 L of TB at 37° at 200 rpm until 0.8 OD, at which point 500 uL of 1M IPTG was added. Cultures were shaken overnight at room temp for protein induction and pelleted at 5000 g for 15 minutes. Cells were lysed (50 mM Tris pH 7.5, 500 mM NaCl, 10 mM MgCl2, 10% glycerol, DTT, lysozyme, protein/phosphatase inhibitors), sonicated for 1 minute (output 4, continuous duty cycle) and spun down at 10,000 g for 1 hour. Supernatant was kept for further purification and cleavage according to manufacturer's protocol using glutathione sepharose beads.

### In-Vitro Kinase Assays:

Cells were trypsinized and normalized for cell number. Cell pellets, or E. coli purified proteins were resuspended in HNE buffer (20 mM HEPES pH 7.4, 100 mM NaCl, 0.5 mM EGTA), and sonicated in the presence of 32P-γ-ATP, and liposomes (10 ug PS, 1 ug PI(4)P in 30 mM HEPES pH 7.4, 1 mM EGTA). Liposome lipids were purchased from Avanti. Reactions were stopped by addition of 50 uL of 4N HCL. To extract lipids, 100 μL of MeOH:CHCl3 (1:1) was added and samples were vortexed 2×30 seconds. Samples were spun down at top speed for 2 min and the organic phase containing phosphatidylinositol lipids (bottom) were separated using thin-layer chromatography (TLC) using 1-propanol: 2N acetic acid (65:35 v/v). TLC plates were prepared ahead of time by coating with 1% Potassium Oxalate. Phosphorylated lipids were visualized by autoradiography on a GE Typhoon FLA 7000 and quantified using ImageQuant TL software.

### GST Pulldown Assays

GST tagged proteins were isolated as described above except that after washing, aliquots of beads with bound protein were added to 293T cell lysates generated by lysis in IP buffer (50 mM Tris-Cl pH 8.0, 150 mM NaCl, 1% NP-40, complete PI/Pis with EDTA) and spinning out DNA at 22,000×g×10 min. These lysates and purified protein were incubated together at 4 degrees for 1 hour before the beads were again pelleted, washed with IP buffer three times, eluted with 2M NaCl, and analyzed by SDS PAGE and western blot.

### Fluorescence Microscopy

293T cells were grown on glass coverslips pre-treated with poly-d-lysine. Adherent cell lines were rinsed with phosphate-buffered saline, pH 7.4 (PBS) and subsequently fixed/permeabilized in −20 MeOH for 20 minutes. After fixation, the cells were blocked for 30 minutes in blocking buffer (PBS with 3% BSA) and labeled with primary antibodies in blocking buffer for 1 hour at room temperature. Coverslips were washed three times with blocking buffer and incubated with Alexa Fluor-conjugated goat secondary antibodies in blocking buffer for 1 hour at room temperature. After incubation with secondary antibodies, coverslips were washed three times with PBS, once with water, and then mounted on a glass microscope slide with Prolong Gold with DAPI. The following primary antibodies were used: LAMP1. Alexa Fluor-conjugated secondary antibodies were used at 1:1000 (Thermo Scientific). Fluorescent and phase contrast images were acquired on a Nikon Eclipse Ti microscope equipped with an Andor Zyla sCMOS camera. Within each experiment, exposure times were kept constant and in the linear range throughout. When using the 60× oil immersion objectives, stacks of images were taken and deconvoluted using AutoQuant.

qRT-PCR

Total RNA was prepared using RNeasy. cDNA was synthesized using Superscript Vilo and qRT-PCR performed utilizing Fast SYBR green and the Realplex Mastercycler. For a list of primers used see oligos in Table 1. Isolation of mRNA and qPCR was performed as follows. 200,000 cells were plated in 6-well plastic dishes. 24 hours later, the RNA in the lysates was extracted using the RNeasy protocol. The RNA was resuspended in 50 μl H₂O at a concentration of 1 μg/μL. cDNA was transcribed using the SuperScript Vilo. The sequences of the oligonucleotides used as primers in the PCR reactions are given in Table 1. The genes that were quantified here were previously shown to be regulated by TFEB.

### Quantification and Statistical Analysis

Experiments were repeated with at least three biological replicates with the following exceptions: Experiments in FIG. 3N-3Q were performed once, and panels with error bars were performed with technical triplicates; 2) Experiments in FIG. 4G-4K were performed once. No samples were excluded from analysis. When comparing two groups, a two-tailed t-test was used. P values are indicated in figure descriptions. When comparing greater than two groups, significance was calculated using ANOVA and Holm-Sidack post-hoc test. Figure descriptions indicate which comparisons are significant, with respective p-values.

TABLE 2
Resources

| | | |
|---|---|---|
| Reagent or Resource | Source | Identifier |

Antibodies

| | | |
|---|---|---|
| PIP4K2A | Cell Signaling | Cat# 5527 |
| | Technologies | |
| PIP4K2B | Cell Signaling | Cat# 9694 |
| | Technologies | |
| PIP4K2C | Proteintech | Cat# 17077-1-AP |
| PIP5K1A | Cell Signaling | Cat# 9693 |
| | Technologies | |
| PIP5K1C | Abcam | Cat# Ab109192 |
| Rac1/2/3 | Cell Signaling | Cat# 2465 |
| | Technologies | |
| Lamp1 | Cell Signaling | Cat# 15665 |
| | Technologies | |
| pAKT-473 | Cell Signaling | Cat# 4060 |
| | Technologies | |
| pAKT-308 | Cell Signaling | Cat# 13038 |
| | Technologies | |
| AKT | Cell Signaling | Cat# 2920 |
| | Technologies | |
| pPRAS40 | Cell Signaling | Cat# 13175 |
| | Technologies | |
| PRAS40 | Cell Signaling | Cat# 2610 |
| | Technologies | |
| Beta-Actin | Abcam | Cat# Ab6276 |
| Tubulin | Abcam | Cat# Ab7291 |
| IRDye-secondary mouse | Licor | Cat# 926-68070 |
| IRDye-secondary rabbit | Licor | Cat# 926-32213 |
| HRP-secondary mouse | Thermo Fisher | Cat# 45000679 |
| | Scientific | |
| HRP-secondary rabbit | Thermo Fisher | Cat# NA934-1ML |
| | Scientific | |
| Lamp1 | Cell Signaling | Cat# CST 15665 |
| | Technologies | |
| Alexa Fluor anti-mouse 568 | Thermo Fisher | Cat# A10037 |
| | Scientific | |
| Alexa Fluor anti-rabbit 488 | Thermo Fisher | Cat# A21206 |
| | Scientific | |

Bacterial and Virus Strains

| | | |
|---|---|---|
| Stbl3 | Life Technologies | Cat# C737303 |
| BL21 | Life Technologies | Cat# C600003 |

Chemicals, Peptides, and Recombinant Proteins

| | | |
|---|---|---|
| RIPA buffer | Sigma | Cat# R0278-500 mL |
| Protease and | Thermo Fisher, | Cat# 88668 |
| phosphatase inhibitor | Pierce | |
| ECL substrate | Thermo Fisher, | Cat# 32106 |
| | Pierce | |
| Triton Lysis buffer | Cell Signaling | Cat# 9803 |
| | Technologies | |
| BCA Assay | Thermo Fisher | Cat# 23225 |
| Precast gels | Life Technologies | Cat# NP0336BOX |
| DMEM | Corning | Cat# 10-013-CV |
| RPMI | Corning | Cat# 10-040-CV |
| Lipofectamine 2000 | Life Technologies | Cat# 11668500 |
| Opti-Mem | Life Technologies | Cat# 31985070 |
| ³H inositol | Perkin Elmer | Cat# |
| | | NET114A005MC |
| ³²ATP | Perkin Elmer | Cat# |
| | | BLU002A001MC |
| Dialyzed FBS | Life Technologies | Cat# 26400044 |
| Inositol free DMEM | Thermo Fisher | Cat# ICN1642954 |
| Glutamine | Life Technologies | Cat# 51985034 |
| Dialyzed FBS | Life Technologies | Cat# 26400044 |
| Inositol free DMEM | Thermo Fisher | Cat# ICN1642954 |
| Glutamine | Life Technologies | Cat# 51985034 |
| Ultima flo scintillation fluid | Perkin Elmer | Cat# 6013599 |
| Anti-HA magnetic beads | Pierce | Cat# 88836 |
| Control magnetic beads | Cell signaling | Cat# 8726 |
| | Technologies | |
| Phosphatidylserine | Avanti | 840032C |
| Phosphatidylinositol-4- | Avanti | Cat# 840151 |
| phosphate | | |
| Phosphatidylinositol-5- | Avanti | Cat# 850152P |
| phosphate | | |
| IPTG | Technova | Cat# I3431 |
| Glutathione Sepharose | Thermo Fisher | Cat# 45000285 |
| TEV protease | Sigma | Cat# T4455-10KU |
| Fast SYBER green | Applied Biosystems | Cat# C0110820 |

Critical Commercial Assays

| | | |
|---|---|---|
| RNEasy | Qiagen | Cat# 74106 |
| SuperScript VILO | Life Technologies | Cat# 11755050 |
| Mastermix | | |
| QuikChange | Agilent | Cat# 200522 |
| BCA protein assay | Thermo Fisher | Cat# 23225 |
| Mycoalert | Lonza | Cat# LT07318 |

Experimental Models: Cell Lines

| | | |
|---|---|---|
| 293T | Clontech | Cat# 632180 |
| HeLa | ATCC | Cat# CCL-2 |
| PaTu 8988t | DSMZ | Cat# ACC162 |
| H1299 | ATCC | Cat# CRL-5803 |
| H1975 | ATCC | Cat# CRL-5908 |
| BJ | ATCC | Cat# CRL-2522 |

Oligonucleotides

| | |
|---|---|
| Table 1 | Herein |

Recombinant DNA

| | | |
|---|---|---|
| LG3GEPIR | Fellmann et al., 2013 | N/A |
| SGEP | Fellmann et al., 2013 | N/A |
| SG3REPIR | This application | N/A |
| SREN | This application | N/A |
| pPIG-3xHA-empty | This application | N/A |
| pPIG-3xHA-PIP4K2A | This application | N/A |
| pPIG-3xHA-PIP4K2A-KD | This application | N/A |
| pPIG-3xHA-PIP4K2C | This application | N/A |
| pPIG 3xHA-PIP4K2C-VD | This application | N/A |

Software and Algorithms

| | | |
|---|---|---|
| AutoQuant | Media Cybernetics | N/A |
| Image Quant TL v8.1 | GE | N/A |
| Prism | Graphpad | N/A |
| Accuri | BD Biosciences | N/A |
| Prism 7 | Graphphad Prism | N/A |
| | Software | |

Other

| | | |
|---|---|---|
| HiChrom Partisphere | VWR | Cat# 101946-560 |
| SAX HPLC 5 micron | | |
| Typhoon FLA 7000 | GE | N/A |

### Example 2: Loss of PIP4K Family Members does not Affect Cell Viability

This Example illustrates that reduction of different PIP4K family members by use of short hairpin RNA (shRNA) inhibitors does not affect cell viability.

To investigate the role of PIP4K enzymes in cellular signaling, tools were generated to systematically deplete individual members of the PIP4K family. First, a series of lentiviral-based tandem miRE-based short hairpin RNAs (shRNAs) were cloned (Tables 1-3) and induced stable knockdown of PIP4K family isoforms in HeLa cells, as well as other human immortalized or transformed cells (FIGS. 1A, 1L-1M; FIGS. 2K-2N) (Fellmann et al., 2013; Fellmann et al., 2011; Pelossof et al., 2017).

TABLE 3
shRNA hairpin/CRISPR guide Sequences Targeting PIP4K Isoforms

| | | | Hairpin/ |
|---|---|---|---|
| Name | Target | Target Sequence | guide |
| Ren | N/A | TAGATAAGCATTATAATTCCTA | hairpin |
| | | (SEQ ID NO: 79) | |
| A650 | PIP4K2A | TATCACATATATTTCAACTCCA | hairpin |
| | | (SEQ ID NO: 80) | |
| A665 | PIP4K2A | TACATTTCTTGTAACTATCACA | hairpin |
| | | (SEQ ID NO: 81) | |
| B130 | PIP4K2B | TTTCTTCTTTGTATCGTATGGC | hairpin |
| | | (SEQ ID NO: 82) | |
| B868 | PIP4K2B | TATTTCTTTAAGATGTTGTGCA | hairpin |
| | | (SEQ ID NO: 83) | |
| C1406 | PIP4K2C | TTGAACATCAGAGAGAACCAGG | hairpin |
| | | (SEQ ID NO: 84) | |
| C3036 | PIP4K2C | TTCTAAAGAGCAATGGTTGCTG | hairpin |
| | | (SEQ ID NO: 85) | |
| C3165 | PIP4K2C | TATTATTATAGTAACAGGAGCA | hairpin |
| | | (SEQ ID NO: 86) | |
| A948 | PIP4K2A/B | TTGATCATCAATTCCAAACCTC | hairpin |
| | | (SEQ ID NO: 87) | |
| g_PIP4K2A-1 | PIP4K2A | TCATCTGGCATCAACATAAC(AGG) | guide |
| | | (SEQ ID NO: 88) | |
| g_PIP4K2A-2 | PIP4K2A | GAATAGGCTTTGAAGTCATC(TGG) | guide |
| | | (SEQ ID NO: 89) | |
| g_PIP4K2B-2 | PIP4K28 | TCATCTGGCATTAGCATGAC(AGG) | guide |
| | | (SEQ ID NO: 90) | |
| g_PIP4K2B-2 | PIP4K2B | GTCCACCTTGATCTTGCTGT(AGG) | guide |
| | | (SEQ ID NO: 91) | |
| g_PIP4K2C | P1P4K2C | ATCTGGCAGCAGCATCACCG(GGG) | guide |
| | | (SEQ ID NO: 92) | |

TABLE 4
Cell Line Annotations

| | | Proteins |
|---|---|---|
| Cell Line | Hairpin/guide IDs | depleted/deleted |
| Ren | Ren | none |
| Ren2 | Ren; Ren | none |
| Ren3 (Ctrl) | Ren; Ren; Ren | none |
| A1 | A650 | PIP4K2A |
| A2 | A665 | PIP4K2A |
| B1 | B130 | PIP4K2B |
| B2 | B868 | PIP4K2B |
| C1 | C1406 | PIP4K2C |
| C2 | C3036 | PIP4K2C |
| AB1 | A650; B868 | PIP4K2A/2B |
| AB2 | A665; B130 | PIP4K2A/2B |
| BC1 | B130; C3165 | PIP4K2B/2C |
| BC2 | B868; C1406 | PIP4K2B/2C |
| AC1 | A650; B868 | PIP4K2A/2C |
| AC2 | A665; B130 | PIP4K2A/2C |
| ABC1 (TKD1) | A650; B130; C1406 | PIP4K2A/2B/2C |
| ABC2 (TKD2) | A665; B868; C3036 | PIP4K2A/2B/2C |
| ABC3 | A948; C3165 | PIP4K2A/2B/2C |
| WT | g_PIP4K2A-1/2B-1/2C-1 | none |
| TKO1 | g_PIP4K2A-2/2B-2/2C-1 | PIP4K2A/2B/2C |
| TKO2 | g_PIP4K2A-1/2B-1/2C-1 | PIP4K2A/2B/2C |
| TKO3 | g_PIP4K2A-1/2B-2/2C-1 | PIP4K2A/2B/2C |

shRNA-mediated silencing of one or all of the PIP4Ks did not result in gross changes in morphology or cause a major change in growth rate of the cells examined (FIG. 1O). As an orthogonal approach, CRISPR/Cas9 was used to delete PIP4K2A, PIP4K2B, and PIP4K2C in HEK293T cells (FIG. 1B, Tables 3-4), with similar results.

### Example 3: Loss of PIP4Ks Results in Increased PI(5)P Levels, Increased PI3K Activation, and Defects in Autophagy

PIP4K2A/B/C triple knockout 293T cells (hereafter TKO) and triple knockdown HeLa cells (hereafter TKD) recapitulated previously reported phenotypes where PIP4K enzymes were depleted. As reported (Gupta et al., 2013), loss of all three enzymes conferred a two-fold increase in their substrate, PI(5)P (FIG. 1C-1D). In addition, TKD cells exhibited increased PI3K signaling upon insulin stimulation (FIG. 1E), consistent with observations of increased insulin sensitivity in Pip4k2b^{-/-} mice (Carricaburu et al., 2003; Lamia et al., 2004). Furthermore, 293T TKO cells accumulated LAMP1 lysosomes, which we attributed to defects in autophagosome-lysosome fusion as described in Pip4k2a^{-/-}, Pip4k2b^{-/-} mouse embryonic fibroblasts (MEFs) (FIG. 1F) (Lundquist et al., 2018).

### Example 4: Cellular PI(4,5)P₂ is Increased Upon Depletion of PIP4K

Interestingly, depletion of the three PIP4Ks in cells altered the levels of phosphoinositides beyond PI(5)P. In both TKD and TKO cells, a surprising increase in PI(4,5)P₂ and concomitant decrease in PI(4)P was observed (FIG. 1G-1J, 2P-2Z). Basal levels of PI(3,4,5)P₃ during growth in culture are typically below the detection limit in HPLC-based assays. However, the inventors were able to detect basal elevation of PI(3,4,5)P₃ in TKO cells, indicating that the increased PI(4,5)P₂ can be utilized by PI3K (FIG. 1K).

PI(4,5)P₂ constitutes about 50% of all phosphoinositides, though the majority of phosphoinositides are bound to cytoskeletal and scaffolding proteins required for cell structure, migration, and adhesion (Brown, 2015; Choi et al., 2015). While PI(4,5)P₂ is highly abundant, local production of PI(4,5)P₂ by PIP5Ks offers spatiotemporal control of signaling through the PI3K and PLC pathways (Choi et al., 2015; Saito et al., 2003; Xie et al., 2009).

### Example 5: PIP4Ks Catalytic-Independent Role in PI(4)P and PI(4,5)P₂ Homeostasis

Members of the PIP4K family have dramatically different catalytic rates. For instance, PIP4Kα (PIP4K2A) has 1000-fold higher activity than PIP4Kγ (PIP4K2), which is considered near-enzymatically-dead (Clarke and Irvine, 2013). Analysis of PI(4,5)P₂ levels in cells with single or double knockdown of PIP4K isoforms revealed an additive effect amongst all three isoforms that does not correlate with their relative catalytic activities (FIG. 2A-2B). Double knockdown of the most active isoforms, PIP4Kα/β, did not phenocopy the triple knockdown, suggesting that catalytic activity is not the most important factor in regulating PI(4,5)P₂ levels. To evaluate whether the catalytic activity of PIP4K enzymes is important for PI(4,5)P₂ regulation, we generated shRNA-resistant HA-tagged kinase-active or kinase-dead PIP4K isoforms (FIG. 2C-2D).

Surprisingly, expression of either kinase-active or kinase-dead (PIP4K2A^{K}) PIP4Ks rescued PI(4,5)P₂ levels (FIG. 2E-2F). Moreover, PI(4)P levels also returned to baseline with expression of either active or kinase-dead PIP4K2A in both TKD and TKO cell lines (FIG. 2G-2H). In contrast, only the active PIP4K2A restored normal PI(5)P levels, indicating that the elevation of this lipid species in knockdown cells, in contrast to PI(4,5)P₂, is due to the loss of PIP4K enzymatic activity (FIG. 2I-2J).

A 3′ IRES-GFP tag was utilized to sort for cell populations with low and high expression of PIP4K2A^{KD} populations: PIP4K2A^{KD_low} and PIP4K2A^{KD_high}, respectively (FIG. 3N). HPLC analysis of these cell lines show a dose-dependent decrease in PI(4,5)P₂ and increase in PI(4)P correlated with levels of PIP4K2A^{KD} expression (FIG. 3O-3P).

These data show that PIP4Ks have a catalytic-independent role in maintaining homeostasis of cellular PI(4)P and PI(4,5)P₂ levels, distinct from their enzymatic function in converting PI(5)P to PI(4,5)P₂.

### Example 6: PIP5K Activity is Elevated in Cells Depleted of PIP4K

The observed increase in PI(4,5)P₂ relative to PI(4)P is consistent with a robust production of PI(4,5)P₂ by the PIP5Ks. PIP5K activity was measured in mechanically disrupted cells. The inventors observed that TKO cells exhibit elevated PIP5K activity, consistent with the model that PIP5K is more active in the absence of PIP4K. The increased PIP5K activity was reversed in cells expressing either active or kinase-dead PIP4K isoforms, indicating it is not dependent on the enzymatic activity of these proteins (FIG. 3A).

All three isoforms of PIP5K are stimulated by phosphatidic acid (PA) and association with G-proteins, such as Rac (Jenkins et al., 1994; Weernink et al., 2004). However, PIP5K activation in PIP4K-depleted cells is not due to these upstream effectors as cells with PIP4K knockdown did not exhibit increased PA levels (FIG. 3D), and knockout of Rac1 did not abrogate the ability of PIP4K to modulate levels of PI(4)P and PI(4,5)P₂. Furthermore, protein levels of PIP5K1A and PIP5K1C were not consistently altered in response to knockdown or knockout of the PIP4K family.

The inventors also considered the possibility that PIP4K has a catalytic-independent role in masking PI(5)P, thereby reducing the availability of adaptor proteins that may inhibit PIP5K activity. To test if increased availability of PI(5)P can enhance PIP5K activity to decrease the PI(4)P to PI(4,5)P₂ ratio, cells were transfected with a transgene encoding inositol phosphate phosphatase (Ipgd), a bacterial 4-phosphatase recognizing PI(4,5)P₂. While transfection of Ipgd into 293T cells caused a dramatic increase in cellular PI(5)P, HPLC analysis of cellular phosphoinositides did not show a concomitant increase in PIP5K activity. The increased availability of PI(5)P in cells with intact PIP4K (WT) did not cause a decreased ratio of PI(4)P to PI(4,5)P₂. In addition, introduction of Ipgd into TKO cells caused a large increase in PI(5)P but did not change the measured PI(4)P or PI(4,5)P₂ levels.

These results indicate that the availability of PI(5)P is not mediating the observed change in PIP5K activity.

### Example 7: PIP4K can Inhibit PIP5K Through Direct Interactions on the Surface of Negatively Charged Membranes

Members of the PIP4K and PIP5K family have been reported to physically interact (Hinchliffe et al., 2002; Huttlin et al., 2017). Such an interaction would be an opportunity for PIP4K to directly inhibit PIP5K. Notably, PIP4K family members are considerably more abundant than PIP5K family members, making the stoichiometric ratio favorable for PIP4Ks to inhibit PIP5Ks (Itzhak et al., 2016; Wisniewski et al., 2014).

To test whether the altered PIP5K activity in PIP4K-depleted cells could be a result of a disrupted physical interaction between these two kinase families, the in vitro kinase activity was measured with purified proteins. As shown in FIG. 3B, all three PIP4K isoforms could directly inhibit the PIP5K2A-catalyzed phosphorylation of PI(4)P into PI(4,5)P₂. To eliminate the possibility that a small molecule contaminant from the PIP4K purifications could inhibit PIP5K1A, PIP4K2C was boiled. As shown in FIG. 3C, this boiled, denatured PIP4K2C could no longer inhibit PIP5K1A. To investigate if the interactions between PIP4K and PIP5K occur at the membrane bilayer, the experiment was repeated with detergent to eliminate the formation of PI(4)P-liposomes. Presenting phosphoinositides in Triton-X100 micelles enhances the activity of some PI kinases, such as the PI4Ks (Guo et al., 2003), but in this case PIP5K1A activity was reduced overall. Importantly, addition of detergent eliminated the ability of PIP4K2A to inhibit PIP5K1A (FIG. 3D). These data indicate that recruitment of both proteins to the membrane surface is required to mediate the inhibitory interaction. This assay was performed using vast molar excess of the substrate PI(4)P relative to the levels of both enzymes, hence the inventors postulated that sequestration of PI(4)P by PIP4Ks is unlikely to account for such inhibition. Given the membrane-dependence of PIP4K and PIP5K association, attempts to co-immunoprecipitate PIP4K with PIP5K in the presence of detergent were unsuccessful. However, FIG. 3F-3G shows interaction of 3×HA-tagged PIP5K1A expressed at near-endogenous levels with endogenous PIP4K2A in an experiment using chemical crosslinking in intact cells. Furthermore, GST-PIP4K2C conjugated glutathione beads pulled down the majority of PIP5K1A in 293T lysates, but only a tiny fraction of PIP5K1C (FIG. 3G-3H), supporting the hypothesis that PIP4Ks interact with PIP5K1A in cells to regulate PI(4,5)P₂ levels.

To further define the nature of this interaction, PIP4K2C variants were generated and assessed their ability to inhibit PIP5K1A activity in vitro. Human PIP4K2A (PDB 2YBX) and PIP4K2C (PDB 2GK9) have been crystallized, while the only PIP5K family member that has been crystallized is from Danio rerio, and it forms a crystal structure similar to that of the PIP4K proteins (PDB 5E3S) (Muftuoglu et al., 2016). Interestingly. PIP4K2C crystallizes as a tetramer via side-by-side interactions of two dimers in which all four catalytic pockets appear on the same side of the positively charged tetramer.

Mutagenesis efforts were focused on PIP4K2C, beginning with a truncation mutant from the COSMIC database carrying a frame shift mutation after aa132 of PIP4K2C (see website at cancer.sanger.ac.uk/cosmic). This truncated mutant, which maintains the residues involved in both dimeric interactions as well as tetrameric interactions was as potent as full length PIP4K2C in its ability to inhibit PIP5K1A activity in vitro (FIG. 4G).

The inventors explored the first 132 residues of PIP4K2C and identified two regions that face the opposing dimer in the tetrameric structure: amino acids 69-75 (VMLLPDD, SEQ ID NO:93) and amino acids 89-95 (FHRENLP, SEQ ID NO:94) (FIG. 3I). Mutating 74DD to be 74NN or mutating 91REN to be 91 EED within GST-PIP4K2C did not prevent this protein from inhibiting PIP5K1A (FIG. 4H). However, introducing mutations into this region that are likely to impair both hydrophobic and charge interactions across the dimer/dimer interaction, (VMLLPDD (SEQ ID NO:93) to EIFLPNN (SEQ ID NO:95)), hereafter named PIP4K2C^{VD}, resulted in a loss of interaction with PIP5K1A (FIG. 3J). Furthermore, PIP4K2C^{VD} failed to inhibit PIP5K1A activity (FIG. 3M) while maintaining PIP4K activity (FIG. 4I-4J), confirming that the protein is folded and functional. To confirm that this interaction between PIP4Ks and PIP5K1A is the physiologic mechanism of increased PI(4,5)P₂ production observed in our CRISPR knockout cells, we reconstituted the TKO cells with PIP4K2C^{VD} or wild type PIP4K2C as previously described (FIG. 4K). Notably, the PIP4K2C^{VD} mutant failed to reverse the decreased PI(4)P and increased PI(4,5)P₂ observed in the triple knockout cells (FIGS. 3L-3M), consistent with a failure to interact with or inhibit PIP5K1A, as the inventors had observed in vitro.

These results reveal a mechanism for regulation of PI(4,5)P₂ production, whereby PIP5K1A is negatively regulated by a direct interaction with the PIP4K N-terminal motif VMLXPDD (SEQ ID NO:96, where X is any amino acid). A fraction of cellular PIP4Ks directly interact with PIP5K enzymes at membranes enriched in PI(4)P. and function to attenuate the PIP5K-mediated conversion of PI(4)P to PI(4,5)P₂.

### Example 8: Structural Role of PIP4K in Regulating PIP5K and PI3K Pathway is Distinct from its Catalytic Role in Autophagy

Levels of PI(4,5)P₂ are remarkably stable to perturbations of its precursor PI(4)P (Nakatsu et al., 2012), yet as illustrated in FIGS. 1G-1J depletion of the PIP4K family members results in nearly two-fold changes in PI(4)P and PI(4,5)P₂. Insulin activation of the PI3K pathway is initiated at the plasma membrane, where conversion of PI(4,5)P₂ into PI(3,4,5)P₃ recruits effector proteins Akt and Pdk1 to propagate signaling cascades.

The inventors hypothesized that, in PIP4K-depleted cells, increased PI(3,4,5)P₃ may be attributed to activation of the PI3K pathway due to elevated production of its substrate, PI(4,5)P₂. Indeed, during acute stimulation, local production of PI(4,5)P₂ by PIP5K1A sustains activation of the PI3K pathway in B cells, keratinocytes, and breast adenocarcinoma (Choi et al., 2016; Saito et al., 2003; Xie et al., 2009).

HPLC analysis of lipids showed that PIP4K knockout cells exhibited a four-fold increase in PI(3,4,5)P₃ upon acute insulin stimulation (FIG. 4A). Consistent with the model that PIP4Ks suppress insulin signaling by their catalytic-independent suppression of PI(4,5)P₂ synthesis by the PIP5Ks, expression of the kinase-dead PIP4K2A or the near-kinase-dead PIP4K2C in TKO cells was able to reverse the insulin-dependent increase in PI(3,4,5)P₃ accumulation. Importantly, expression of the PIP4K2C^{VD} mutant failed to rescue PI(3,4,5)P₃ levels (FIG. 4A), consistent with the VMLXPDD (SEQ ID NO:96) motif mediating negative regulation of the PI3K pathway.

Further, HeLa TKD cells displayed enhanced PI3K pathway activation, as judged by S473 phosphorylation of AKT as well as AKT-mediated phosphorylation of PRAS40 (Manning and Toker, 2017) (FIGS. 4B-4C). Expression of either PIP4K2A^{KD} or PIP4K2C reversed the enhanced insulin sensitivity whether measured by PI(3,4,5)P₃ levels or AKT activation. The results provided herein indicate that increasing PIP5K activity by eliminating PIP4K isoforms drives overproduction of PI(4,5)P₂ in plasma membrane pools that may be used by PI3K during insulin signaling.

The inventors then asked whether the non-catalytic function of PIP4Ks was required for the previously reported role of PIP4K2A in mediating autophagy (Lundquist et al., 2018). Loss of PIP4Ks resulted in accumulation of LAMP1-positive lysosomes and transcriptional increases in genes targeted by TFEB (FIGS. 4D-4F). However, unlike the changes observed in phosphoinositides, only reconstitution with active, but not kinase-dead, PIP4K2A showed restored regulation of autophagy (FIGS. 4D-4F). Together, this work highlights the complexity and multi-functionality of PIP4K enzymes.

### REFERENCES

Balla, T. (2013). Phosphoinositides: tiny lipids with giant impact on cell regulation. Physiol Rev 93, 1019-1137.
Bondeson, D. P., and Crews, C. M. (2017). Targeted Protein Degradation by Small Molecules. Annu Rev Pharmacol Toxicol 57, 107-123.
Brown, D. A. (2015). PIP2 Clustering: From model membranes to cells. Chem Phys Lipids 192, 33-40.
Bultsma, Y., Keune, W. J., and Divecha, N. (2010). PIP4Kbeta interacts with and modulates nuclear localization of the high-activity PtdIns5P-4-kinase isoform PIP4Kalpha. Biochem J 430, 223-235.
Carricaburu, V., Lamia, K. A., Lo, E., Favereaux, L., Payrastre, B., Cantley, L. C., and Rameh, L. E. (2003). The phosphatidylinositol (PI)-5-phosphate 4-kinase type II enzyme controls insulin signaling by regulating PI-3,4,5-trisphosphate degradation. Proc Natl Acad Sci USA 100, 9867-9872.
Choi, S., Hedman, A. C., Sayedyahossein, S., Thapa, N., Sacks, D. B., and Anderson, R. A. (2016). Agonist-stimulated phosphatidylinositol-3,4,5-trisphosphate generation by scaffolded phosphoinositide kinases. Nat Cell Biol 18, 1324-1335.
Choi, S., Thapa, N., Tan, X., Hedman, A. C., and Anderson, R. A. (2015). PIP kinases define PI4,5P(2)signaling specificity by association with effectors. Biochim Biophys Acta 1851, 711-723.
Clarke, J. H., Emson, P. C., and Irvine, R. F. (2008). Localization of phosphatidylinositol phosphate kinase Ilgamma in kidney to a membrane trafficking compartment within specialized cells of the nephron. Am J Physiol Renal Physiol 295, F1422-1430.
Clarke, Jonathan H., and Irvine, Robin F. (2013). Evolutionarily conserved structural changes in phosphatidylinositol 5-phosphate 4-kinase (PI5P4K) isoforms are responsible for differences in enzyme activity and localization. Biochemical Journal 454, 49-57.
De Leo, M. G., Staiano, L., Vicinanza, M., Luciani, A., Carissimo, A., Mutarelli, M., Di Campli, A., Polishchuk, E., Di Tullio, G., Morra, V., et al. (2016). Autophagosome-lysosome fusion triggers a lysosomal response mediated by TLR9 and controlled by OCRL. Nat Cell Biol 18, 839-850.
Emerling, B. M., Hurov, J. B., Poulogiannis, G., Tsukazawa, K. S., Choo-Wing, R., Wulf. G. M., Bell, E. L., Shim, H. S., Lamia, K. A., Rameh, L. E., et al. (2013).
Depletion of a putatively druggable class of phosphatidylinositol kinases inhibits growth of p53-null tumors. Cell 155, 844-857.
Fellmann, C., Hoffmann, T., Sridhar, V., Hopfgartner, B., Muhar, M., Roth, M., Lai, D. Y., Barbosa, I. A., Kwon, J. S., Guan, Y., et al. (2013). An optimized microRNA backbone for effective single-copy RNAi. Cell Rep 5, 1704-1713.
Fellmann, C., Zuber, J., McJunkin, K., Chang, K., Malone, C. D., Dickins, R. A., Xu, Q., Hengartner, M. O., Elledge, S. J., Hannon, G. J., et al. (2011). Functional identification of optimized RNAi triggers using a massively parallel sensor assay. Mol Cell 41, 733-746.
Fruman, D. A., Chiu, H., Hopkins, B. D., Bagrodia, S., Cantley, L. C., and Abraham, R. T. (2017). The PI3K Pathway in Human Disease. Cell 170, 605-635.
Guo, J., Wenk, M. R., Pellegrini, L., Onofri, F., Benfenati, F., and De Camilli, P. (2003). Phosphatidylinositol 4-kinase type ilalpha is responsible for the phosphatidylinositol 4-kinase activity associated with synaptic vesicles. Proc Natl Acad Sci USA 100, 3995-4000.
Gupta, A., Toscano, S., Trivedi, D., Jones, D. R., Mathre, S., Clarke, J. H., Divecha, N., and Raghu, P. (2013). Phosphatidylinositol 5-phosphate 4-kinase (PIP4K) regulates TOR signaling and cell growth during Drosophila development. Proc Natl Acad Sci USA 110, 5963-5968.
Hinchliffe, K. A., Ciruela, A., Letcher, A. J., Divecha, N., and Irvine, R. F. (1999). Regulation of type IIalpha phosphatidylinositol phosphate kinase localisation by the protein kinase CK2. Curr Biol 9, 983-986.
Hinchliffe, K. A., Giudici, M. L., Letcher, A. J., and Irvine, R. F. (2002). Type IIa phosphatidylinositol phosphate kinase associates with the plasma membrane via interaction with type I isoforms. Biochem J 363, 563-570.
Homma, K., Terui, S., Minemura, M., Qadota, H., Anraku, Y., Kanaho, Y., and Ohya, Y. (1998). Phosphatidylinositol-4-phosphate 5-kinase localized on the plasma membrane is essential for yeast cell morphogenesis. Journal of Biological Chemistry 273, 15779-15786.
Huttlin. E. L., Bruckner, R. J., Paulo, J. A., Cannon, J. R., Ting, L., Baltier, K., Colby, G., Gebreab, F., Gygi, M. P., Parzen, H., et al. (2017). Architecture of the human interactome defines protein communities and disease networks. Nature 545, 505-509.
Idevall-Hagren, O., and De Camilli, P. (2015). Detection and manipulation of phosphoinositides. Biochim Biophys Acta 1851, 736-745.
Itoh, T., Ijuin, T., and Takenawa, T. (1998). A novel phosphatidylinositol-5-phosphate 4-kinase (phosphatidylinositol-phosphate kinase IIgamma) is phosphorylated in the endoplasmic reticulum in response to mitogenic signals. J Biol Chem 273, 20292-20299.
Itzhak, D. N., Tyanova, S., Cox, J., and Borner, G. H. (2016). Global, quantitative and dynamic mapping of protein subcellular localization. Elife 5.
Jefferies, H. B., Cooke, F. T., Jat, P., Boucheron, C., Koizumi, T., Hayakawa, M., Kaizawa, H., Ohishi, T., Workman, P., Waterfield, M. D., et al. (2008). A selective PlKfyve inhibitor blocks Ptdlns(3,5)P(2) production and disrupts endomembrane transport and retroviral budding. EMBO Rep 9, 164-170.
Jenkins, G. H., Fisette, P. L., and Anderson, R. A. (1994). Type I phosphatidylinositol 4-phosphate 5-kinase isoforms are specifically stimulated by phosphatidic acid. J Biol Chem 269, 11547-11554.
Jones, D. R., Bultsma, Y., Keune, W. J., Halstead, J. R., Elouarrat, D., Mohammed, S., Heck, A. J., D'Santos, C. S., and Divecha, N. (2006). Nuclear PtdIns5P as a transducer of stress signaling: an in vivo role for PIP4Kbeta. Mol Cell 23, 685-695.
Kunz, J., Fuelling, A., Kolbe, L., and Anderson, R. A. (2001). Stereo-specific Substrate Recognition by Phosphatidylinositol Phosphate Kinases Is Swapped by Changing a Single Amino Acid Residue. Journal of Biological Chemistry 277, 5611-5619.
Kunz, J., Wilson, M. P., Kisseleva, M., Hurley, J. H., Majerus, P. W., and Anderson, R. A. (2000a). The Activation Loop of Phosphatidylinositol Phosphate Kinases Determines Signaling Specificity. Mol Cell 5, 1-11.
Kunz, J., Wilson, M. P., Kisseleva, M., Hurley, J. H., Majerus, P. W., and Anderson, R. A. (2000b). The activation loop of phosphatidylinositol phosphate kinases determines signaling specificity. Mol Cell 5, 1-11.
Lamia, K. A., Peroni. O. D., Kim, Y. B., Rameh, L. E., Kahn, B. B., and Cantley. L. C. (2004). Increased insulin sensitivity and reduced adiposity in phosphatidylinositol 5-phosphate 4-kinase beta−/− mice. Mol Cell Biol 24, 5080-5087.
Lundquist, M. R., Goncalves, M. D., Loughran. R. M., Possik, E., Vijayaraghavan, T., Yang, A., Pauli, C., Ravi, A., Verma, A., Yang, Z., et al. (2018). Phosphatidylinositol-5-Phosphate 4-Kinases Regulate Cellular Lipid Metabolism By Facilitating Autophagy. Molecular Cell 70, 531-544.e539.
Mackey. A. M., Sarkes, D. A., Bettencourt, I., Asara, J. M., and Rameh, L. E. (2014). PIP4kgamma is a substrate for mTORC1 that maintains basal mTORC1 signaling during starvation. Sci Signal 7, ra104.
Manning, B. D., and Toker, A. (2017). AKT/PKB Signaling: Navigating the Network. Cell 169, 381-405.
Muftuoglu, Y., Xue, Y., Gao, X., Wu, D., and Ha, Y. (2016). Mechanism of substrate specificity of phosphatidylinositol phosphate kinases. Proc Natl Acad Sci USA 113, 8711-8716.
Nakatsu, F., Baskin, J. M., Chung. J., Tanner, L. B., Shui, G., Lee, S. Y., Pirruccello, M., Hao, M., Ingolia, N. T., Wenk, M. R., et al. (2012). PtdIns4P synthesis by PI4KIIIalpha at the plasma membrane and its impact on plasma membrane identity. J Cell Biol 199, 1003-1016.
Niebuhr, K., Giuriato, S., Pedron, T., Philpott, D. J., Gaits, F., Sable, J., Sheetz, M. P., Parsot, C., Sansonetti, P. J., and Payrastre, B. (2002). Conversion of Ptdlns(4,5)P(2) into PtdIns(5)P by the S. flexneri effector IpgD reorganizes host cell morphology. EMBO J 21, 5069-5078.
Pelossof, R., Fairchild, L., Huang, C. H., Widmer. C., Sreedharan, V. T., Sinha, N., Lai, D. Y., Guan, Y., Premsrirut, P. K., Tschaharganeh, D. F., et al. (2017). Prediction of potent shRNAs with a sequential classification algorithm. Nat Biotechnol 35, 350-353.
Pendaries, C., Tronchere, H., Arbibe, L., Mounier, J., Gozani, O., Cantley, L. C., Fry, M. J., Gaits-Iacovoni, F., Sansonetti, J., and Payrastre, B. (2006). Ptdlns(5)P activates the host cell PI3-kinase/Akt pathway during Shigella flexneri infection. EMBO J 25, 1024-2034.
Rameh, L. E., Tolias, K. F., Duckworth, B. C., and Cantley, L. C. (1997). A new pathway for synthesis of phosphatidylinositol-4,5-bisphosphate. Nature 390, 192-196.
Ran, F. A., Hsu, P. D., Wright. J., Agarwala, V., Scott, D. A., and Zhang, F. (2013). Genome engineering using the CRISPR-Cas9 system. Nat Protoc 8, 2281-2308.
Saito, K., Tolias, K. F., Saci, A., Koon, H. B., Humphries, L. A., Scharenberg, A., Rawlings, D. J., Kinet, J. P., and Carpenter, C. L. (2003). BTK regulates Ptdlns-4,5-P2 synthesis: importance for calcium signaling and PI3K activity. Immunity 19, 669-678.
Shim, H., Wu, C., Ramsamooj, S., Bosch, K. N., Chen, Z., Emerling, B. M., Yun, J., Liu, H., Choo-Wing, R., Yang, Z., et al. (2016). Deletion of the gene Pip4k2c, a novel phosphatidylinositol kinase, results in hyperactivation of the immune system. Proc Natl Acad Sci USA 113, 7596-7601.
Sun, Y., Thapa, N., Hedman, A. C., and Anderson, R. A. (2013). Phosphatidylinositol 4,5-bisphosphate: targeted production and signaling. Bioessays 35, 513-522.
van den Bout, I., and Divecha, N. (2009). PIP5K-driven Ptdlns(4,5)P2 synthesis: regulation and cellular functions. J Cell Sci 122, 3837-3850.
Wang, H., Sun, H.-Q., Zhu, X., Zhang, L., Albanesi, J., Levine, B., and Yin, H. (2015). GABARAPs regulate PI4P-dependent autophagosome:lysosome fusion. Proceedings of the National Academy of Sciences, 201507263.
Weernink, P. A., Meletiadis, K., Hommeltenberg, S., Hinz, M., Ishihara, H., Schmidt, M., and Jakobs, K. H. (2004). Activation of type I phosphatidylinositol 4-phosphate 5-kinase isoforms by the Rho GTPases, RhoA, Rac1, and Cdc42. J Biol Chem 279, 7840-7849.
Wilcox, A., and Hinchliffe, K. A. (2008). Regulation of extranuclear PtdIns5P production by phosphatidylinositol phosphate 4-kinase 2alpha. FEBS Lett 582, 1391-1394.
Wisniewski, J. R., Hein, M. Y., Cox, J., and Mann, M. (2014). A “proteomic ruler” for protein copy number and concentration estimation without spike-in standards. Mol Cell Proteomics 13, 3497-3506.
Xie, Z., Chang, S. M., Pennypacker, S. D., Liao, E. Y., and Bikle, D. D. (2009). Phosphatidylinositol-4-phosphate 5-kinase 1alpha mediates extracellular calcium-induced keratinocyte differentiation. Mol Biol Cell 20, 1695-1704.
Zolov, S. N., Bridges, D., Zhang, Y., Lee, W. W., Riehle, E., Verma, R., Lenk, G. M., Converso-Baran, K., Weide, T., Albin, R. L., et al. (2012). In vivo, Pikfyve generates PI(3,5)P2, which serves as both a signaling lipid and the major precursor for PI5P. Proc Natl Acad Sci USA 109, 17472-17477.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements:

1. A method comprising depleting, degrading, or inhibiting one or more isoforms of phosphatidylinositol-5-phosphate 4-kinases (PIP4Ks) in a subject.
2. The method of statement 1, which reduces the onset or severity of diabetes, metabolic syndrome, insulin resistance, obesity, cancer, immune deficiency, autoimmune disease, infection, or a combination thereof.
3. The method of statement 1 or 2, which enhances insulin signaling in the subject.
4. The method of statement 1, 2, or 3, wherein depleting, degrading, or inhibiting one or more isoforms of phosphatidylinositol-5-phosphate 4-kinases (PIP4Ks) reduces scaffolding or interaction of one or more the isoforms with at least one other PIP4K or PIP5K.
5. The method of statement 1-3 or 4, wherein one or more of the isoforms of phosphatidylinositol-5-phosphate 4-kinase is PIP4K2A, PIP4K2B, or PIP4K2C.
6. The method of statement 1-4 or 5, wherein degrading one or more isoforms of phosphatidylinositol-5-phosphate 4-kinases (PIP4Ks) comprises deleting a sequence comprising SEQ ID NO:5 or 96 within one or more isoforms of phosphatidylinositol-5-phosphate 4-kinases (PIP4Ks).
7. The method of statement 1-4 or 5, wherein degrading one or more isoforms of phosphatidylinositol-5-phosphate 4-kinases (PIP4Ks) comprises contacting a binding moiety with one or more of the isoforms, wherein the binding moiety is directly or indirectly linked to an agent that signals cells to degrade a phosphatidylinositol-5-phosphate 4-kinase bound to the agent.
8. The method of statement 7, wherein the agent that signals cells to degrade a phosphatidylinositol-5-phosphate 4-kinase bound to the agent is an E3 ubiquitin ligase.
9. The method of statement 7 or 8, wherein the binding moiety is a small molecule, an antibody, a peptide, a polysaccharide, or a lipid that binds specifically to one of the isoforms of phosphatidylinositol-5-phosphate 4-kinase (PIP4K).
10. The method of statement 7, 8, or 9, wherein the binding moiety is indirectly linked to the agent via hydrogen bonding, hydrophobic interaction, steric interaction, hydrophilic interaction, or a combination thereof.
11. The method of statement 7-9 or 10, wherein indirectly linked means that interaction between the binding moiety and the agent occurs before the binding moiety is contacted with one of the isoforms of phosphatidylinositol-5-phosphate 4-kinase (PIP4K).
12. The method of statement 7-10 or 11, wherein indirectly linked means that interaction between the binding moiety and the agent occurs after the binding moiety is contacted with one of the isoforms of phosphatidylinositol-5-phosphate 4-kinase (PIP4K).
13. The method of statement 1-11 or 12, wherein degrading one or more isoforms of phosphatidylinositol-5-phosphate 4-kinases (PIP4Ks) comprises contacting one or more of the isoforms with an antibody specific for one of the isoforms of phosphatidylinositol-5-phosphate 4-kinase (PIP4K), wherein the antibody has an Fc domain that can bind an E3 ubiquitin ligase.
14. The method of statement 1-12 or 13, wherein inhibiting one or more isoforms of phosphatidylinositol-5-phosphate 4-kinases (PIP4Ks) comprises inhibiting structural interaction between at least one the isoforms with endogenous cellular structures or proteins.
15. The method of statement 1-13 or 14, wherein inhibiting one or more isoforms of phosphatidylinositol-5-phosphate 4-kinases (PIP4Ks) comprises (a) administering an inhibitor of the one or more isoforms of phosphatidylinositol-5-phosphate 4-kinases or (b) modifying one or more phosphatidylinositol-5-phosphate 4-kinase gene sequences.
16. The method of statement 1-14 or 15, wherein inhibiting one or more isoforms of phosphatidylinositol-5-phosphate 4-kinases (PIP4Ks) comprises inhibiting expression or function of one or more isoforms of phosphatidylinositol-5-phosphate 4-kinases (PIP4Ks).
17. The method of statement 16, wherein inhibiting expression or function of one or more isoforms of phosphatidylinositol-5-phosphate 4-kinases (PIP4Ks) comprises administering an antibody, nucleic acid inhibitor, or small molecule inhibitor of one or more phosphatidylinositol-5-phosphate 4-kinase isoforms.
18. The method of statement 1-14 or 15, wherein inhibiting one or more isoforms of phosphatidylinositol-5-phosphate 4-kinases (PIP4Ks) comprises contacting the one or more isoforms of phosphatidylinositol-5-phosphate 4-kinases (PIP4Ks) with a PIP4K peptide comprising SEQ ID NO:1-5 or 96, wherein the PIP4K peptide is not a full-length PIP4K polypeptide.
19. The method of statement 18, wherein the PIP4K peptide does not have a catalytic site.
20. The method of statement 18, wherein the PIP4K peptide is less than 400, less than 350, less than 300, less than 250, less than 200, less than 150, or less than 100 amino acids in length.
21. The method of statement 18, wherein the PIP4K peptide is at least 20, at least 25, at least 30, at least 35, at least 40, at least 45, or at least 50 amino acids in length.
22. The method of statement 1-20 or 21, wherein one or more of the phosphatidylinositol-5-phosphate 4-kinases is PIP4K2A, PIP4K2B, or PIP4K2C.
23. The method of statement 1-21 or 22, wherein the phosphatidylinositol-5-phosphate 4-kinase is PIP4K2B and the subject has or is suspected of having diabetes, metabolic syndrome, insulin resistance, obesity, or a combination thereof.
24. The method of statement 2-22 or 23, wherein the infection is a bacterial infection, viral infection, fungal infection, or a combination thereof.
25. The method of statement 1-23 or 24, wherein the phosphatidylinositol-5-phosphate 4-kinase is PIP4K2C and the subject has or is suspected of having cancer, immune deficiency, autoimmune disease, infection, or a combination thereof.
26. The method of statement 7-24 or 25, wherein the binding moiety binds with specificity to one or more PIP4K2A, PIP4K2B, or PIP4K2C proteins.
27. The method of statement 7-25 or 26, wherein the binding moiety binds with specificity to an epitope having sequence with at least 95% sequence identity to a 5-amino acid to 30 amino acid portions of SEQ ID NO:6, 8, or 10.
28. The method of statement 7-26 or 27, wherein the binding moiety binds with specificity to an epitope having sequence with at least 95% sequence identity to SEQ ID NO:1, 2, 3, 4, 5, or 96.
29. The method of statement 16-27 or 28, wherein inhibiting expression or function comprises contacting a nucleic acid encoding the one or more isoforms of phosphatidylinositol-5-phosphate 4-kinases (PIP4Ks) with a small hairpin RNA, an siRNA, or a vector that can express a small hairpin RNA or an siRNA.
30. The method of statement 29, wherein the nucleic acid binds to an RNA with at least 95% sequence identity or complementarity to SEQ ID NO:7, 8, or 11.
31. The method of statement 1-29 or 30, wherein degrading one or more isoforms of phosphatidylinositol-5-phosphate 4-kinases (PIP4Ks) comprises CRISPR-mediated, TALENS-mediated, or ZFN-mediated knockout or knockdown of one or more of PIP4K2A, PIP4K2B, or PIP4K2C.
32. The method of statement 31, comprising isolating a population of cells from the subject and incubating the cells with one or more CRISPR, TALENS, or ZFN reagents to generate a modified population of cells with one or more modified phosphatidylinositol-5-phosphate 4-kinase gene sequences.
33. The method of statement 32, wherein the one or more CRISPR, TALENS, or ZFN reagents comprises one or more guide RNAs or a vector that can express one or more guide RNAs, where the one or more of the guide RNAs can specifically bind to a PIP4K2A, PIP4K2B, or PIP4K2C genomic site.
34. A method comprising negatively regulating PIP5K1A or PI3K comprising contacting the PIP5K1A with a peptide comprising a SEQ ID NO:1-5 or 96 (where X is any amino acid), wherein the peptide is not a wild type phosphatidylinositol-5-phosphate 4-kinase.
35. A method comprising regulating PIP5K1A or PI3K comprising contacting the PIP5K1A or PI3K with a phosphatidylinositol-5-phosphate 4-kinase comprising a mutation in sequence SEQ ID NO:1-5 or 96 (where X is any amino acid).
36. The method of statement 34 or 35, wherein the peptide or the phosphatidylinositol-5-phosphate 4-kinase comprises an intact phosphatidylinositol-5-phosphate 4-kinase catalytic site.
37. A method comprising administering an inhibitor of PIP4K interaction with an endogenous PIP4K or endogenous PIP5K to a subject.
38. The method of statement 37, wherein the inhibitor comprises a PIP4K binding moiety, a PIP4K peptide comprising sequence SEQ ID NO:1-5 or 96, or a small molecule.
39. A kit comprising one or more binding moieties that specifically binds to at least one phosphatidylinositol-5-phosphate 4-kinase, and instructions for administering one or more of the binding moieties, wherein the binding moiety is directly or indirectly linked to an agent that signals cells to degrade a phosphatidylinositol-5-phosphate 4-kinase bound to the agent.
40. The kit of statement 39, wherein the agent that signals cells to degrade a phosphatidylinositol-5-phosphate 4-kinase bound to the agent is an E3 ubiquitin ligase.
41. The kit of statement 39 or 40, further comprising the agent that signals cells to degrade a phosphatidylinositol-5-phosphate 4-kinase.
42. The kit of statement 39, 40 or 41, wherein the binding moiety is a small molecule, an antibody, a peptide, a polysaccharide, or a lipid that binds specifically to one of the isoforms of phosphatidylinositol-5-phosphate 4-kinase (PIP4K).
43. The kit of statement 39-41 or 42, wherein the binding moiety is indirectly linked to the agent via hydrogen bonding, hydrophobic interaction, steric interaction, hydrophilic interaction, or a combination thereof.
44. The kit of statement 39-42 or 43, wherein indirectly linked means that interaction between the binding moiety and the agent occurs before the binding moiety is contacted with one of the isoforms of phosphatidylinositol-5-phosphate 4-kinase (PIP4K).
45. The kit of statement 39-43 or 44, wherein indirectly linked means that interaction between the binding moiety and the agent occurs after the binding moiety is contacted with one of the isoforms of phosphatidylinositol-5-phosphate 4-kinase (PIP4K).
46. The kit of statement 39-44 or 45, wherein degrading one or more isoforms of phosphatidylinositol-5-phosphate 4-kinases (PIP4Ks) comprises contacting one or more of the isoforms with an antibody specific for one of the isoforms of phosphatidylinositol-5-phosphate 4-kinase (PIP4K), wherein the antibody has an Fc domain that can bind an E3 ubiquitin ligase.
47. A kit comprising components that include one or more sterile implements for isolating cells from a subject, reagents for culturing cells, one or more guide RNA(s) for targeting one or more genomic PIP4K sites, implements for administering modified cells back into the subject, and any combination thereof.
48. The kit of statement 47, further comprising instructions for using the components to modify genomic PIP4K sites and thereby inhibit PIP4K activity in the subject.
49. A method comprising knockdown or knockout of one or more phosphatidylinositol-5-phosphate 4-kinases (PIP4Ks) in a population of mammalian cells to generate a population of modified mammalian cells with reduced expression or function of one or more phosphatidylinositol-5-phosphate 4-kinase.
50. The method of statement 49, wherein one or more of the phosphatidylinositol-5-phosphate 4-kinases is PIP4K2A, PIP4K2B, or PIP4K2C.
51. The method of statement 49 or 50, comprising knockout of one or more phosphatidylinositol-5-phosphate 4-kinases (PIP4Ks) in the population of mammalian cells.
52. The method of statement 49, 50, or 51, further comprising administering the population of modified mammalian cells to a subject.
53. The method of statement 52, wherein the population of modified mammalian cells is allogenic or autologous to the subject.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method comprising degrading, modifying, or inhibiting one or more phosphatidylinositol-5-phosphate 4-kinase (PIP4K) isoform(s) in a mammalian subject, or in a population of mammalian cells, to generate a subject with a population of modified mammalian cells, or a population of modified mammalian cells.

2. The method of claim 1, wherein one or more of the phosphatidylinositol-5-phosphate 4-kinase is PIP4K2A, PIP4K2B, or PIP4K2C.

3. The method of claim 1, wherein the phosphatidylinositol-5-phosphate 4-kinase (PIP4K) is PIP4K2B and the subject has, or is suspected of having, diabetes, metabolic syndrome, insulin resistance, obesity, or a combination thereof.

4. The method of claim 1, wherein the phosphatidylinositol-5-phosphate 4-kinase (PIP4K) is PIP4K2C and the subject has, or is suspected of having, cancer, immune deficiency, autoimmune disease, infection, or a combination thereof.

5. The method of claim 1, which inhibits interaction of one or more phosphatidylinositol-5-phosphate 4-kinase (PIP4K) isoforms with one or more endogenous PIP4Ks or phosphatidylinositol-4-phosphate 5-kinases in a mammalian subject, or in a population of mammalian cells, to generate a subject with a population of modified mammalian cells, or a population of modified mammalian cells.

6. The method of claim 1, which reduces scaffolding or interaction of one or more the isoforms with at least one other phosphatidylinositol-5-phosphate 4-kinase (PIP4K) or phosphatidylinositol-4-phosphate 5-kinase (PIP5K).

7. The method of claim 1, which modulates PIP5K activity, PI3K activity, or a combination thereof.

8. The method of claim 1, which comprises administering to a mammalian subject a phosphatidylinositol-5-phosphate 4-kinase comprising a mutation in sequence SEQ ID NO:1-5 or 96, where X is any amino acid.

9. The method of claim 8, wherein the peptide has an intact phosphatidylinositol-5-phosphate 4-kinase (PIP4K) catalytic site.

10. The method of claim 1, wherein degrading, modifying, or inhibiting one or more phosphatidylinositol-5-phosphate 4-kinase (PIP4K) isoform(s) in a mammalian subject, or in a population of mammalian cells, does not block or inhibit the phosphatidylinositol-5-phosphate 4-kinase (PIP4K) isoform(s) catalytic site.

11. The method of claim 1, wherein degrading, modifying, or inhibiting one or more phosphatidylinositol-5-phosphate 4-kinase (PIP4K) isoform(s) comprises contacting or administering a binding moiety to the one or more of the PIP4K isoforms.

12. The method of claim 11, wherein the binding entity binds with specificity to one or more PIP4K2A, PIP4K2B, or PIP4K2C proteins.

13. The method of claim 11, wherein the binding moiety is directly or indirectly linked to an agent that signals cells to degrade a phosphatidylinositol-5-phosphate 4-kinase bound to the agent.

14. The method of claim 11, wherein the binding entity binds with specificity to an epitope having sequence with at least 95% sequence identity to a 5-amino acid to 30 amino acid portion of SEQ ID NO:6, 8, or 10.

15. The method of claim 11, wherein the binding entity binds with specificity to an epitope having sequence with at least 95% sequence identity to SEQ ID NO:1, 2, 3, 4, 5, 93, or 96.

16. The method of claim 1, wherein degrading, modifying, or inhibiting one or more isoforms of phosphatidylinositol-5-phosphate 4-kinases (PIP4Ks) comprises inhibiting structural interaction between at least one the phosphatidylinositol-5-phosphate 4-kinase (PIP4K) isoforms with endogenous cellular structures or proteins.

17. The method of claim 1, wherein inhibiting one or more isoforms of phosphatidylinositol-5-phosphate 4-kinases (PIP4Ks) comprises (a) administering an inhibitor of the one or more isoforms of phosphatidylinositol-5-phosphate 4-kinases or (b) modifying one or more phosphatidylinositol-5-phosphate 4-kinase gene sequences.

18. The method of claim 1, wherein inhibiting one or more isoforms of phosphatidylinositol-5-phosphate 4-kinases (PIP4Ks) comprises inhibiting expression or function of one or more isoforms of phosphatidylinositol-5-phosphate 4-kinases (PIP4Ks).

19. The method of claim 18, wherein inhibiting expression or function of one or more isoforms of phosphatidylinositol-5-phosphate 4-kinases (PIP4Ks) comprises administering an antibody, nucleic acid inhibitor, or small molecule inhibitor of one or more phosphatidylinositol-5-phosphate 4-kinase isoforms.

20. The method of claim 19, wherein the inhibitor is a small hairpin RNA, an siRNA, or a vector that can express a small hairpin RNA or an siRNA.

21. The method of claim 19, wherein the inhibitor is a nucleic acid that binds to an RNA with at least 95% sequence identity or complementarity to SEQ ID NO:7, 8, or 11.

22. The method of claim 19, wherein the inhibitor is a binding entity binds with specificity to a non-catalytic site of one or more PIP4K2A, PIP4K2B, or PIP4K2C protein.

23. The method of claim 1, wherein modifying one or more phosphatidylinositol-5-phosphate 4-kinase gene sequences comprises CRISPR-mediated, TALENS-mediated, or ZFN-mediated knockout or knockdown of one or more of PIP4K2A, PIP4K2B, or PIP4K2C.

24. The method of claim 23, comprising isolating a population of cells from the subject and incubating the cells with one or more CRISPR, TALENS, or ZFN reagents to generate a modified population of cells with one or more modified phosphatidylinositol-5-phosphate 4-kinase gene sequences.

25. The method of claim 24, wherein the one or more CRISPR, TALENS, or ZFN reagents comprises one or more guide RNAs or a vector that can express one or more guide RNAs, where one or more of the guide RNAs can specifically bind to a PIP4K2A, PIP4K2B, or PIP4K2C genomic site.

26. The method of claim 1, wherein degrading, modifying, or inhibiting one or more isoforms of phosphatidylinositol-5-phosphate 4-kinases (PIP4Ks) comprises the inhibiting one or more isoforms of phosphatidylinositol-5-phosphate 4-kinases (PIP4Ks) contacting or administering to the subject a PIP4K peptide comprising SEQ ID NO:1-5 or 96, wherein the peptide is not a full-length PIP4K.

27. A kit comprising one or more binding moieties that specifically binds to at least one phosphatidylinositol-5-phosphate 4-kinase, and instructions for administering one or more of the binding moieties, wherein the binding moiety is directly or indirectly linked to an agent that signals cells to degrade a phosphatidylinositol-5-phosphate 4-kinase bound to the agent.

28. Use of a phosphatidylinositol-5-phosphate 4-kinase comprising a mutation in sequence VMLXPDD (SEQ ID NO:96, where X is any amino acid), for treatment of diabetes, metabolic syndrome, insulin resistance, obesity, cancer, or a combination thereof.

29. Use of antibody, nucleic acid inhibitor, or small molecule inhibitor of one or more phosphatidylinositol-5-phosphate 4-kinase isoforms for treatment of diabetes, metabolic syndrome, insulin resistance, obesity, cancer, or a combination thereof.


SEQUENCE LISTING (96 sequences)

SEQ ID NO: 1 - protein, 26 aa, Homo sapiens
INELSHVQIPVMLMPDDFKAYSKIKV

SEQ ID NO: 2 - protein, 26 aa, Homo sapiens
INELSNVPVPVMLMPDDFKAYSKIKV

SEQ ID NO: 3 - protein, 26 aa, Homo sapiens
INELSQVPPPVMLLPDDFKASSKIKV

SEQ ID NO: 4 - protein, 26 aa, Homo sapiens
INELSQVPPPEIFLPNNFKASSKIKV

SEQ ID NO: 5 - protein, 7 aa, Homo sapiens
  SITE 4: Xaa = any amino acid
VMLXPDD

SEQ ID NO: 6 - protein, 406 aa, Homo sapiens
MATPGNLGSSVLASKTKTKKKHFVAQKVKLFRASDPLLSVLMWGVNHSINELSHVQIPVMLMPDDFKAYSKIKVDNHLFNKENMPSHFKFKEYCPMVFRNLRERFGIDDQDFQNSLTRSAPLPNDSQARSGARFHTSYDKRYIIKTITSEDVAEMHNILKKYHQYIVECHGITLLPQFLGMYRLNVDGVEIYVIVTRNVFSHRLSVYRKYDLKGSTVAREASDKEKAKELPTLKDNDFINEGQKIYIDDNNKKVFLEKLKKDVEFLAQLKLMDYSLLVGIHDVERAEQEEVECEENDGEEEGESDGTHPVGTPPDSPGNTLNSSPPLAPGEFDPNIDVYGIKCHENSPRKEVYFMAIIDILTHYDAKKKAAHAAKTVKHGAGAEISTVNPEQYSKRFLDFIGHILT

SEQ ID NO: 7 - DNA, 3820 bp, Homo sapiens
agccgcgggctgagcgcggatccgcggcgggcgcaggatacgggccggggcgcgagccgagcgcagtctgccgggccgagcgggcggagcgagccgagtggggctgagcgcgccggcggcggcgggcggagcggagcgcggcgcgccggggccgccgccggggggatgcggctgcctccccgggccggggtgtagagagggcgggtccccggcctcgggagcacggcggtggaggggacataggaggcggccatggcgacccccggcaacctagggtcctctgtcctggcgagcaagaccaagaccaagaagaagcacttcgtagcgcagaaagtgaagctgtttcgggccagcgacccgctgctcagcgtcctcatgtggggggtaaaccactcgatcaatgaactgagccatgttcaaatccctgttatgttgatgccagatgacttcaaagcctattcaaaaataaaggtggacaatcacctttttaacaaagaaaacatgccgagccatttcaagtttaaggaatactgcccgatggtcttccgtaacctgcgggagaggtttggaattgatgatcaagatttccagaattccctgaccaggagcgcacccctccccaacgactcccaggcccgcagtggagctcgttttcacacttcctacgacaaaagatacatcatcaagactattaccagtgaagacgtggccgaaatgcacaacatcctgaagaaataccaccagtacatagtggaatgtcatgggatcacccttcttccccagttcttgggcatgtaccggcttaatgttgatggagttgaaatatatgtgatagttacaagaaatgtattcagccaccgtttgtctgtgtataggaaatacgacttaaagggctctacagtggctagagaagctagtgacaaagaaaaggccaaagaactgccaactctgaaagataatgatttcattaatgagggccaaaagatttatattgatgacaacaacaagaaggtcttcctggaaaaactaaaaaaggatgttgagtttctggcccagctgaagctcatggactacagtctgctggtgggaattcatgatgtggagagagccgaacaggaggaagtggagtgtgaggagaacgatggggaggaggagggcgagagcgatggcacccacccggtgggaacccccccagatagccccgggaatacactgaacagctcaccacccctggctcccggggagttcgatccgaacatcgacgtctatggaattaagtgccatgaaaactcgcctaggaaggaggtgtacttcatggcaattattgacatccttactcattatgatgcaaaaaagaaagctgcccatgctgcaaaaactgttaaacatggcgctggcgcggagatctccaccgtgaacccagaacagtattcaaagcgctttttggactttattggccacatcttgacgtaacctcctgcgcagcctcggacagacatgaacattggatggacagaggtggcttcggtgtaggaaaaatgaaaaccaaactcagtgaagtactcatcttgcaggaagcaaacctccttgtttacatcttcaggccaagatgactgatttgggggctactcgctttacagctacctgattttcccagcatcgttctagctatttctgactttgtgtatatgtgtgtgtgtgtgtgttgggggggggtgagtgtgtgcgcgcgtgtgcattttaaaagtcataaattaattaaaacagatccacttcggtcagtatgtgtcccaacaaagaccctttgattccagctatggccgaatgaatgagtgagtgagtgagtgagtgaatgaacacacgtgtgggggaggggagaaggaagtgcatgatgtcaggcaccgtgttggcatcacacaacaaactgtggatcagttttttttttttttttttttttttggagttgaaagatgtgagacagtattcagaataatgaagataataatgatgattattataataatgatgatgattccaaggaaaaaacctacagcgaatgttccatttctaccccgcacgcagacactctccctaacactgataacctgagcccccagcactggacggaagaatgctggcgtctccgtgtgtactggttcagggttctggccccagccttgtcaggaccccctggtgtccagagcccccacccctcccgcaacaagcagctgatgccccagtgattctctatacatttttcacctcggccaatatgtccaggaaaactgcttacttctcttttcttgcctggagccttcattgttcacccttacgttgcaatataggaattaatgctacaaaataaaagtaaagcttacctgaaaagtgcatagtttggggcaatggtatctacatctcccactgtgggaaaaccagcaaagcatcaaaactctcaattctcctgttaccaaatgcagatctgaattataagatgtttatgtttgaccattgtttcaacaatgggattttgttacgaattatccctttaactgaaaccctcagttttactgtttacattattaggaaaacagggatatcttttgaatctaaaaatttgatgtacagcatgtgatttttgaagtttacatgtaaagtcacagtataggtgaaataacgtttgtcatattttgagacgtatcctgcagccatgtttttacgtgagtgttttagtcaaagtacatggtagacagtctttcacaataaaaggaaaaggatttttttttcctccaaatgtacatttatcaacctaatgattgatttttttaaaaagagatttcgccccagtctggtttatgaaagttcattgccctaaactgtgctgattgtttttaatcaagttataaatttccaacctagatcatgtatctaccaactctcctgcattttccaaaaggcattgagcttaaatattagtcttgcttagagtaggttatccacttacatgctgcgctaaagccatgcctttgaaactccttgtttaaaacatgatatgatttttgtgggcagtttcagaaaagaaaacaaacaaacaaaaatcgaccctttaattattacttgcaactcaacagatctccctgccgtactgccttttccaggaactttacttcagggctgtccagattgcagctgtgccccgtgtatgtggatctagttcacagagtctttggaagccagcagtcgtgccctccgtatactgtccactcattttatgtagatttggtatcctcagcagccagtgttaacaccactgtcacgtagtgtacagattcatcttttatgtatttaaagtaatccatactatgatttggtttttccctgcaccattaattctggcatcagatcagtttttgtgttgtgaagttctactgtggtttgacccaagaccacaaccatgagaccctgaagtaaagataaggtacacatacattatttgagtaactgtttccttgggggccaatctgtgtatgcttttagaagtttacagaatgcttttatttttgtctataacaaacagtctgtcatttatttctgttgataaaccatttggacagagtgaggacgtttgccctgttatctcctagtgctaacaatacactccagtcatgagccgggctttacaaataaagcacttttgatgactcacaagatgaatccttttttcctctgtcccaattgtgtgtctctgttccaaacacattttaaatactcggtcctgacagtgtctttagctaatccttgaagaaatgaaagtggaattgaa

SEQ ID NO: 8 - protein, 416 aa, Homo sapiens
MSSNCTSTTAVAVAPLSASKTKTKKKHFVCQKVKLFRASEPILSVLMWGVNHTINELSNVPVPVMLMPDDFKAYSKIKVDNHLFNKENLPSRFKFKEYCPMVFRNLRERFGIDDQDYQNSVTRSAPINSDSQGRCGTRFLTTYDRRFVIKTVSSEDVAEMHNILKKYHQFIVECHGNTLLPQFLGMYRLTVDGVETYMVVTRNVFSHRLTVHRKYDLKGSTVAREASDKEKAKDLPTFKDNDFLNEGQKLHVGEESKKNFLEKLKRDVEFLAQLKIMDYSLLVGIHDVDRAEQEEMEVEERAEDEECENDGVGGNLLCSYGTPPDSPGNLLSFPRFFGPGEFDPSVDVYAMKSHESSPKKEVYFMAIIDILTPYDTKKKAAHAAKTVKHGAGAEISTVNPEQYSKRFNEFMSNILT

SEQ ID NO: 9 - DNA, 5732 bp, Homo sapiens
ttgcgggaaagagccaaaccctggcgttggggggcccgggcggggagcccctcccgcggtccacagcgacgcctgcccagccctcctccccttccggctccggcacggggccccgaggcgttcggaggccaggcgggtttctgtcaggcccggggaggaggggcgggcggggcggccgctgcctccccgggacgggccgtaccacgcggacggggaggacggggccaggggactgcagggcggctgcaccgcccgggggcggggtgcggagcgggccggcgggctccccggggcggggcgggagggcggggcgtggggcggacggaaccaccggggcggggtgggaggtaacgggacgggcgcgaccatggcgcggtgagggagcgggggtggggatcggtccgggggaggcctgaggccgctggcttgtgcgctgtctccgccgcccccctctttcgccgccgccgccgccgccccgggcatgtcgtccaactgcaccagcaccacggcggtggcggtggcgccgctcagcgccagcaagaccaagaccaagaagaagcatttcgtgtgccagaaagtgaagctattccgggccagcgagccgatcctcagcgtcctgatgtggggggtgaaccacacgatcaatgagctgagcaatgttcctgttcctgtcatgctaatgccagatgacttcaaagcctacagcaagatcaaggtggacaatcatctcttcaataaggagaacctgcccagccgctttaagtttaaggagtattgccccatggtgttccgaaaccttcgggagaggtttggaattgatgatcaggattaccagaattcagtgacgcgcagcgcccccatcaacagtgacagccagggtcggtgtggcacgcgtttcctcaccacctacgaccggcgctttgtcatcaagactgtgtccagcgaggacgtggcggagatgcacaacatcttaaagaaataccaccagtttatagtggagtgtcatggcaacacgcttttgccacagttcctgggcatgtaccgcctgaccgtggatggtgtggaaacctacatggtggttaccaggaacgtgttcagccatcggctcactgtgcatcgcaagtatgacctcaagggttctacggttgccagagaagcgagcgacaaggagaaggccaaggacttgccaacattcaaagacaatgacttcctcaatgaagggcagaagctgcatgtgggagaggagagtaaaaagaacttcctggagaaactgaagcgggacgttgagttcttggcacagctgaagatcatggactacagcctgctggtgggcatccacgacgtggaccgggcagagcaggaggagatggaggtggaggagcgggcagaggacgaggagtgtgagaatgatggggtgggtggcaacctactctgctcctatggcacacctccggacagccctggcaacctcctcagctttcctcggttctttggtcctggggaattcgacccctctgttgacgtctatgccatgaaaagccatgaaagttcccccaagaaggaggtgtatttcatggccatcattgatatcctcacgccatacgatacaaagaagaaagctgcacatgctgccaaaacggtgaaacacggggcaggggccgagatctcgactgtgaaccctgagcagtactccaaacgcttcaacgagtttatgtccaacatcctgacgtagttctcttctaccttcagccagagccagagagctggatatggggtcggggatcgggagttagggagaagggtgtatttgggctagatgggagggtgggagcagagtcgggtttgggagggctttagcaatgagactgcagcctgtgacaccgaaagagactttagctgaagaggagggggatgtgctgtgtgtgcacctgctcacaggatgtaaccccaccttctgcttacccttgattttttctccccatttgacacccaggttaaaaaggggttccctttttggtaccttgtaaccttttaagataccttggggctagagatgacttcgtgggtttatttgggttttgtttctgaaatttcattgctccaggtttgctatttataatcatatttcatcagcctacccaccctccccatctttgctgagctctcagttcccttcaattaaagagatacccggtagacccagcacaagggtccttccagaaccaagtgctatggatgccagattggagaggtcagacacctcgccctgctgcatttgctcttgtctggattaactttgtaatttatggagtattgtgcacaacttcctccacctttcccttggattcaagtgaaaactgttgcattattcctccatcctgtctggaatacaccaggtcaacaccagagatctcagatcagaatcagagatctcagaggggaataagttcatcctcatgggatggtgaggggcaggaaagcggctgggctcttggacacctggttctcagagaaccctgtgatgatcacccaagccccaggctgtcttagcccctggagttcagaagtcctctctgtaaagcctgcctcccactaggtcaagaggaactagagtacctttggatttatcaggaccctcatgtttaaatggttatttccctttgggaaaacttcagaaactgatgtatcaaatgaggccctgtgccctcgatctatttccttcttccttctgacctcctcccaggcactcttacttctagccgaactcttagctctgggcagatctccaagcgcctggagtgctttttagcagagacacctcgttaagctccgggatgaccttgtaggagatctgtctccctgtgcctggagagttacagccagcaaggtgcccccatcttagagtgtggtgtccaaacgtgaggtggcttcctagttacatgaggatgtgatccaggaaatccagtttggaggcttgatgtgggttttgacctggcctcagccttggggctgtttttccttgttgccccgctctagacttttagcagatctgcagcccacaggcttttttggaaggagtggcttcctgcaggtgttccacctgccttcggagcctgccacccaggccctcagaactgagccacaggctgctctggccaggagagaaacagctctgttgttctgcattgggggaggtacattcctgcatcttctcaccccctcaaccaggaactggggatttgggatgagatatggtcagacttgtagataaccccaaagatgtgaagatcgcttgtgaaaccattttgaatgaatagattggtttcctgtggctccctccaaacctggccaagcccagcttccgaagcaggaaccagcactgtctctgtgcctgactcacagcatataggtcaggaaagaatggagacggcattcttggacttcactggggctgctggattggatgggaaaccttctggaagaggcagatgggggtcaaaccactgccttggccccaggaaggggccataggtaggtctgaacaactgccgcaagaccactacatgacttagggaacttgaaaccaactggctcatggagaaaacaaatttgacttgggaaagggattatgtaggaataatgtttggacttgatttccccacgtcataatgaagaatggaagtttggatctgctcctcgtcaggcgcagcatctctgaagcttggaaagctgtcttccagcagcctccgtggcctcgggttcctaccggcttctctgcatttggtctgctgatcatgttgccataatgtgtatggaaagtgtaacacattcttactggttaaagacgactaccaggtatctaacttgtttaacattgagtttgtgtgtgtgtgtgtatgtttgtgtgttttgtatattgtttacattttgagaggtagcattctgtttcaaatgctttttgtttttctgacagtattgttgactgggtcataacattttgagctgtggtttggtggattttcaatttttttttttaaaggtcattcgctgtgctatcttcaaaaccttgagtttggcccccaatttttggcattcaaatgtttaaaagctatttatcttggtttatacaagtttcctttctcttctttttgtcatggtattctatttggtctgcagtttgaatgtagagaaagtggactgatcccccaagcgttgtctgcccccactctttcctccttgggtcccgccattcttttactgggcagtcgagggcattggaggggaagtgactgccctcagcctcactccctggggccatgaagaaaagctaaacagtctcatggcatctcagaataatgttgggtctcccaagaagaaaggtgtaagaataacgacatggctgattaggcgaggccaggatagggctaaggccaggattcctggctggcatccagtcaccccttctcccatccttccccctcttcttccacaagtccgcagccgagacactgtagtctcccagccacagtgatgagtgccctggagactccactgacctctagatgaaggcccctggccctggttcctgttaattaacctctgggtctttgagtcccccagcacaaacttctttcctgtaccctgcggcttggggtcacagggcatgccgggaagccacagctgaggggcgcagactgaagcagtgctccacctctccttctttagctcaggggttgctggtctgtggcaggcgccacgagtggcccctgtggctgttctcagtggcagtctcttaagttcccaccacaggcagctctttatcccctctccctacttgactctttctcttgcctgtgcttttggcctcaaacaggcctgctggtagcgctcagggcgtgaggctacactcctgccctgcctttcctgtcttcatggtctgccagggcataccttggggaggtggaccaaagacccaggactttttgcagtagccagtcctaccccccagttgtctttttaccaattcagggtgggagagaaaactgcagcaccccagcatgtgagttactcaggtgttgggggctagaagggacagtgcgtttaaacaacactcagagctctggccttaaacctgtggccccccaagtctaggagcctcatctcttcctggcagtcatgcgggcaggaggtcctgaaagggaaaacccattcagacaactgttccccaatctaccagccatctgcaggggtcagtgaccgtggccctctccctcctctagaatgtgccacttatgaagagtgccccatggggaaaaggagactcagctgtcccttggcagcttgtgccagtatcccagggcagaagtttccacaggagcctcttgcccttgcgcagagccactgtgagaggcggtgggagccaacacccttgggggagggggcagtactgctcggcacatcccagcatcaggtcagatcattgaaattaaaaaatgtgaattaagttcatatccaccttttggggaagcaggacaaaccaccaccccaccaagtgtgtgacttctccatatcccactgcagtttccattttttaaatgggaattttcaatcccctgtgcttgtctaacgtctgctttaaaaagtttgagaccctgttactgtttgaaaatgcatgcatgttacgatgaatctccaacctgaggaaaaaaataaaactcaaaaagctttgtgta

SEQ ID NO: 10 - protein, 421 aa, Homo sapiens
MASSSVPPATVSAATAGPGPGFGFASKTKKKHFVQQKVKVFRAADPLVGVFLWGVAHSINELSQVPPPVMLLPDDFKASSKIKVNNHLFHRENLPSHFKFKEYCPQVFRNLRDRFGIDDQDYLVSLTRNPPSESEGSDGRFLISYDRTLVIKEVSSEDIADMHSNLSNYHQYIVKCHGNTLLPQFLGMYRVSVDNEDSYMLVMRNMFSHRLPVHRKYDLKGSLVSREASDKEKVKELPTLKDMDFLNKNQKVYIGEEEKKIFLEKLKRDVEFLVQLKIMDYSLLLGIHDIIRGSEPEEEAPVREDESEVDGDCSLTGPPALVGSYGTSPEGIGGYIHSHRPLGPGEFESFIDVYAIRSAEGAPQKEVYFMGLIDILTQYDAKKKAAHAAKTVKHGAGAEISTVHPEQYAKRFLDFITNIFA

SEQ ID NO: 11 - DNA, 3176 bp, Homo sapiens
ggtcacgtgacagcagcgcaggtgagcgccgcttccggggtcgggcgcctggatagctgccggctccggcttccacttggtcggttgcgcgggagactatggcgtcctcctcggtcccaccagccacggtatcggcggcgacagcaggccccggcccaggtttcggcttcgcctccaagaccaagaagaagcatttcgtgcagcagaaggtgaaggtgttccgggcggccgacccgctggtgggtgtgttcctgtggggcgtagcccactcgatcaatgagctcagccaggtgcctcccccggtgatgctgctgccagatgactttaaggccagctccaagatcaaggtcaacaatcaccttttccacagggaaaatctgcccagtcatttcaagttcaaggagtattgtccccaggtcttcaggaacctccgtgatcgatttggcattgatgaccaagattacttggtgtcccttacccgaaacccccccagcgaaagtgaaggcagtgatggtcgcttccttatctcctacgatcggactctggtcatcaaagaagtatccagtgaggacattgctgacatgcatagcaacctctccaactatcaccagtacattgtgaagtgccatggcaacacgcttctgccccagttcctggggatgtaccgagtcagtgtggacaacgaagacagctacatgcttgtgatgcgcaatatgtttagccaccgtcttcctgtgcacaggaagtatgacctcaagggttccctagtgtcccgggaagccagcgataaggaaaaggttaaagaattgcccacccttaaggatatggactttctcaacaagaaccagaaagtatatattggtgaagaggagaagaaaatatttctggagaagctgaagagagatgtggagtttctagtgcagctgaagatcatggactacagccttctgctaggcatccacgacatcattcggggctctgaaccagaggaggaagcgcccgtgcgggaggatgagtcagaggtggatggggactgcagcctgactggacctcctgctctggtgggctcctatggcacctccccagagggtatcggaggctacatccattcccatcggcccctgggcccaggagagtttgagtccttcattgatgtctatgccatccggagtgctgaaggagccccccagaaggaggtctacttcatgggcctcattgatatccttacacagtatgatgctaagaagaaagcagctcatgcagccaaaactgtcaagcatggggctggggcagagatctctactgtccatccggagcagtatgctaagcgattcctggattttattaccaacatctttgcctaagagactgcctggttctctctgatgttcaaggtggtggggttctgagacacttgggggaattgtggggatattctagccaccagttctcttcttcctttgctaaattcaggctgcaggctccttccatccagataactccatcctgtcgagtaggctctttctgaccctcagaaatacattgtcctttttcctctttgcccatttttcttccctctcttcctccccatgagaagtctgcttgtagtattagaatgttattgttgactctctcccaagtgccttgatctttgtaatatctcctgttgtttctatgatataggagctaggggaagggggttgtttgccttcttcaggacctgactggacagatggacctggctcaagcaactactctggatgcactttgctgtgtgggatgaactaaaagtgtctgaattttgctgataactttataaaactcactatggcatgcttccctcctggtgggccctaggatggatgacactcaagatactacagatgtgggtgcaggcatgcacacacacgatggaatatggccattcctacacaggtggggtagagagtgggtcagcagcctggcacctcacagaggtgggacctaagaggactcatgattatgcagagaattggattgggtctctgtcatagattgagtaatctcttcccttacctcaattccatctccacccatctctacatctgggcacagcaacccagagatggccaaaagcattcaagcctgggggaagatgtttgactattgctgctcttcaccagaacctcacacctctcctgggactggaacccttcagtgggtgtgtggccagttttggaggctggaatgatgggccagggtgtaggattcattctccatgtaaagtttcctttcatcctgcctagccatccccaaggtttatttccagaagaaaggaatatctctacttggatcaattctggtcatttcaagaggatggaggcctcaagtgtgggaacttcccctactccctggatgtgtgtacctagcacacttccttctcccacccctttttccagttggatttgtttttctgttctcttctgtcctgtcttatactgcaactgtgtctcctaggggacagatggccttctttgtcatcttcactctccacccccagagaggagtcagagccataactcaatcactcagcccctccaaagatagttgatgtgtgataatctcataatgttgagaaccctgatgagatacattgtcttcctctccctacaatgcctctggggccaaggcacccattcttcttgctatcctccatcccccttgaggcttccactttttttttttttagacataaagctgggcatcagcaactggcctgtggtgatgcaaagctgctttgctctgtatctggctggactgatctgtctcacaagaagccatgaggccatagggagaagctccctctccccttcatcttctgctccaaaggtggtagcaagaggagtacccagttaggggttggagcccccatataacatcttcctgtcagaagactgatggatctttttcattccaaccatctccctttcccccgatgaatgcaataaaactctgtgacaccagcaaccattgctctttagaaatgggttttctgatcatatggctgatgtgttatgggcagtatggatgtcttcatttgttgcttctgtttttcatcttttttgttttattaataaaaatttatgtatttgctcctgttactataataatacagggaataaattattcaatccaaa

SEQ ID NO: 12 - protein, 491 aa, Homo sapiens
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 13 - protein, 64 aa, Homo sapiens
MCNTNMSVPTDGAVTTSQIPASEQETLVRPKPLLLKLLKSVGAQKDTYTMKEFATKHRAKNIPV

SEQ ID NO: 14 - protein, 60 aa, Homo sapiens
MCNTNMSVPTDGAVTTSQIPASEQETLVRPKPLLLKLLKSVGAQKDTYTMKENHRTQVHL

SEQ ID NO: 15 - protein, 73 aa, Homo sapiens [sequence omitted]

SEQ ID NO: 16 - protein, 497 aa, Homo sapiens
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 17 - protein, 618 aa, Homo sapiens
MHKTASQRLFPGPSYQNIKSIMEDSTILSDWTNSNKQKMKYDFSCELYRMSTYSTFPAGVPVSERSLARAGFYYTGVNDKVKCFCCGLMLDNWKLGDSPIQKHKQLYPSCSFIQNLVSASLGSTSKNTSPMRNSFAHSLSPTLEHSSLFSGSYSSLSPNPLNSRAVEDISSSRTNPYSYAMSTEEARFLTYHMWPLTFLSPSELARAGFYYIGPGDRVACFACGGKLSNWEPKDDAMSEHRRHFPNCPFLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSTSDTTGEENADPPIIHFGPGESSSEDAVMMNTPVVKSALEMGFNRDLVKQTVQSKILTTGENYKTVNDIVSALLNAEDEKREEEKEKQAEEMASDDLSLIRKNRMALFQQLTCVLPILDNLLKANVINKQEHDIIKQKTQIPLQARELIDTILVKGNAAANIFKNCLKEIDSTLYKNLFVDKNMKYIPTEDVSGLSLEEQLRRLQEERTCKVCMDKEVSVVFIPCGHLVVCQECAPSLRKCPICRGIIKGTVRTFLS

SEQ ID NO: 18 - protein, 298 aa, Homo sapiens
MGPKDSAKCLHRGPQPSHWAAGDGPTQERCGPRSLGSPVLGLDTCRAWDHVDGQILGQLRPLTEEEEEEGAGATLSRGPAFPGMGSEELRLASFYDWPLTAEVPPELLAAAGFFHTGHQDKVRCFFCYGGLQSWKRGDDPWTEHAKWFPSCQFLLRSKGRDFVHSVQETHSQLLGSWDPWEEPEDAAPVAPSVPASGYPELPTPRREVQSESAQEPGGVSPAEAQRAWWVLEPPGARDVEAQLRRLQEERTCKVCLDRAVSIVFVPCGHLVCAECAPGLQLCPICRAPVRSRVRTFLS

SEQ ID NO: 19 - protein, 213 aa, Homo sapiens
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 20 - protein, 172 aa, Homo sapiens
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRVYTLKERCLQVVRSLVKPENYRRLDIVRSLYEDLEDHPNVQKDLERLTQERIAHQRMGD

SEQ ID NO: 21 - protein, 193 aa, Homo sapiens
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRVLMTPVGQFCVVPALVENTFLLGRLTDAKTGTSQGHVGAGRADRVWRGKLTYLPAGRWRGCGCVVSVKEHFPEKEESRME

SEQ ID NO: 22 - protein, 442 aa, Homo sapiens [sequence omitted]

SEQ ID NO: 23 - protein, 441 aa, Homo sapiens
MAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 24 - protein, 379 aa, Homo sapiens
MEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 25 - protein, 398 aa, Homo sapiens
MAGEGDQQDAAHNMGNHLPLLPAESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFRYAWTVAQCKICASHIGWKFTATKKDMSPQKFWGLTRSALLPTIPDTEDEISPDKVILCL

SEQ ID NO: 26 - protein, 475 aa, Homo sapiens [sequence omitted]

SEQ ID NO: 27 - DNA, 22 bp, Artificial Sequence
  A synthetic oligonucleotide
tgtttgaatgaggcttcagtac

SEQ ID NO: 28 - DNA, 43 bp, Artificial Sequence
  A synthetic oligonucleotide
aggcgcgaagactcaattgaaggctcgagaaggtatattgctg

SEQ ID NO: 29 - DNA, 40 bp, Artificial Sequence
  A synthetic oligonucleotide
cacttttttcaattgacacgtacgcgtattctaccgggta

SEQ ID NO: 30 - DNA, 20 bp, Artificial Sequence
  A synthetic oligonucleotide
agagtggatgggcaagaagc

SEQ ID NO: 31 - DNA, 22 bp, Artificial Sequence
  A synthetic oligonucleotide
aagatggagtcattgctgttca

SEQ ID NO: 32 - DNA, 21 bp, Artificial Sequence
  A synthetic oligonucleotide
gattgactctccctcaccact

SEQ ID NO: 33 - DNA, 22 bp, Artificial Sequence
  A synthetic oligonucleotide
ctgtgtacaagagcagaggttc

SEQ ID NO: 34 - DNA, 20 bp, Artificial Sequence
  A synthetic oligonucleotide
tgcttgagctcaggacagtg

SEQ ID NO: 35 - DNA, 20 bp, Artificial Sequence
  A synthetic oligonucleotide
actaagaccaagatggggcc

SEQ ID NO: 36 - DNA, 20 bp, Artificial Sequence
  A synthetic oligonucleotide
gctggtgtgggcagattgct

SEQ ID NO: 37 - DNA, 21 bp, Artificial Sequence
  A synthetic oligonucleotide
cactgctacagcctcacactg

SEQ ID NO: 38 - DNA, 20 bp, Artificial Sequence
  A synthetic oligonucleotide
gattgcctgcattcgctctg

SEQ ID NO: 39 - DNA, 20 bp, Artificial Sequence
  A synthetic oligonucleotide
atgctgctgtttggatgggt

SEQ ID NO: 40 - DNA, 21 bp, Artificial Sequence
  A synthetic oligonucleotide
gttctcatggcatctccaagg

SEQ ID NO: 41 - DNA, 22 bp, Artificial Sequence
  A synthetic oligonucleotide
gactgttgtgagcatgaagttc

SEQ ID NO: 42 - DNA, 21 bp, Artificial Sequence
  A synthetic oligonucleotide
gagggcctatttcccatgatt

SEQ ID NO: 43 - DNA, 43 bp, Artificial Sequence
  A synthetic oligonucleotide
ccagctgaagctcatgaactacagtctgctggtgggaattcat

SEQ ID NO: 44 - DNA, 43 bp, Artificial Sequence
  A synthetic oligonucleotide
atgaattcccaccagcagactgtagttcatgagcttcagctgg

SEQ ID NO: 45 - DNA, 47 bp, Artificial Sequence
  A synthetic oligonucleotide
taggaaggaggtgtacttcatggcaattattaacatccttactcatt

SEQ ID NO: 46 - DNA, 47 bp, Artificial Sequence
  A synthetic oligonucleotide
aatgagtaaggatgttaataattgccatgaagtacacctccttccta

SEQ ID NO: 47 - DNA, 43 bp, Artificial Sequence
  A synthetic oligonucleotide
acagctgaagatcatgaactacagcctgctggtgggcatccac

SEQ ID NO: 48 - DNA, 43 bp, Artificial Sequence
  A synthetic oligonucleotide
gtggatgcccaccagcaggctgtagttcatgatcttcagctgt

SEQ ID NO: 49 - DNA, 41 bp, Artificial Sequence
  A synthetic oligonucleotide
gtatttcatggccatcattaatatcctcacgccatacgata

SEQ ID NO: 50 - DNA, 41 bp, Artificial Sequence
  A synthetic oligonucleotide
tatcgtatggcgtgaggatattaatgatggccatgaaatac

SEQ ID NO: 51 - DNA, 44 bp, Artificial Sequence
  A synthetic oligonucleotide
agtgcagctgaagatcatgaactacagccttctgctaggcatcc

SEQ ID NO: 52 - DNA, 44 bp, Artificial Sequence
  A synthetic oligonucleotide
ggatgcctagcagaaggctgtagttcatgatcttcagctgcact

SEQ ID NO: 53 - DNA, 46 bp, Artificial Sequence
  A synthetic oligonucleotide
ccagaaggaggtctacttcatgggcctcattgatatccttacacag

SEQ ID NO: 54 - DNA, 46 bp, Artificial Sequence
  A synthetic oligonucleotide
ctgtgtaaggatatcaatgaggcccatgaagtagacctccttctgg

SEQ ID NO: 55 - DNA, 86 bp, Artificial Sequence
  A synthetic oligonucleotide
gcatgtaccggcttaatgttgatggtgtagagatttacgtcattgtaactaggaacgtattcagccaccgtttgtctgtgtatagg

SEQ ID NO: 56 - DNA, 86 bp, Artificial Sequence
  A synthetic oligonucleotide
cctatacacagacaaacggtggctgaatacgttcctagttacaatgacgtaaatctctacaccatcaacattaagccggtacatgc

SEQ ID NO: 57 - DNA, 58 bp, Artificial Sequence
  A synthetic oligonucleotide
cttccgtaacctgcgggaaagattcggtatagacgaccaagatttccagaattccctg

SEQ ID NO: 58 - DNA, 58 bp, Artificial Sequence
  A synthetic oligonucleotide
cagggaattctggaaatcttggtcgtctataccgaatctttcccgcaggttacggaag

SEQ ID NO: 59 - DNA, 57 bp, Artificial Sequence
  A synthetic oligonucleotide
cattgatatcctcaccccttatgacactaaaaaaaaagctgcacatgctgccaaaac

SEQ ID NO: 60 - DNA, 57 bp, Artificial Sequence
  A synthetic oligonucleotide
gttttggcagcatgtgcagctttttttttagtgtcataaggggtgaggatatcaatg

SEQ ID NO: 61 - DNA, 57 bp, Artificial Sequence
  A synthetic oligonucleotide
ccagcgaggacgtggcggagatgcataatatattgaaaaagtaccaccagtttatag

SEQ ID NO: 62 - DNA, 57 bp, Artificial Sequence
  A synthetic oligonucleotide
ctataaactggtggtactttttcaatatattatgcatctccgccacgtcctcgctgg

SEQ ID NO: 63 - DNA, 36 bp, Artificial Sequence
  A synthetic oligonucleotide
ggttatgctgctgccgaacaatttcaaggcgagcag

SEQ ID NO: 64 - DNA, 36 bp, Artificial Sequence
  A synthetic oligonucleotide
ctgctcgccttgaaattgttcggcagcagcataacc

SEQ ID NO: 65 - DNA, 50 bp, Artificial Sequence
  A synthetic oligonucleotide
atttgaagtggctcggcaggtcttcctcgtgaaacaggtggttgttaacc

SEQ ID NO: 66 - DNA, 50 bp, Artificial Sequence
  A synthetic oligonucleotide
ggttaacaaccacctgtttcacgaggaagacctgccgagccacttcaaat

SEQ ID NO: 67 - DNA, 46 bp, Artificial Sequence
  A synthetic oligonucleotide
agccaggtgccgccgccggagatattcctgccgaacaatttcaagg

SEQ ID NO: 68 - DNA, 46 bp, Artificial Sequence
  A synthetic oligonucleotide
ccttgaaattgttcggcaggaatatctccggcggcggcacctggct

SEQ ID NO: 69 - DNA, 19 bp, Artificial Sequence
  A synthetic oligonucleotide
accttggggcagtagtgag

SEQ ID NO: 70 - DNA, 21 bp, Artificial Sequence
  A synthetic oligonucleotide
tcccgctgtttctgaatacca

SEQ ID NO: 71 - DNA, 22 bp, Artificial Sequence
  A synthetic oligonucleotide
gcatgacaccgctaaagaaaac

SEQ ID NO: 72 - DNA, 23 bp, Artificial Sequence
  A synthetic oligonucleotide
cacaacgtgatgattatgtgggt

SEQ ID NO: 73 - DNA, 22 bp, Artificial Sequence
  A synthetic oligonucleotide
attcagggcgagtacatgatcc

SEQ ID NO: 74 - DNA, 19 bp, Artificial Sequence
  A synthetic oligonucleotide
cgacaccttgagcgtgtag

SEQ ID NO: 75 - DNA, 22 bp, Artificial Sequence
  A synthetic oligonucleotide
cacgagaaatgcaacacgttac

SEQ ID NO: 76 - DNA, 23 bp, Artificial Sequence
  A synthetic oligonucleotide
gggtgccactaacacatctgtat

SEQ ID NO: 77 - DNA, 26 bp, Artificial Sequence
  A synthetic oligonucleotide
atagcacagcctggatagcaacgtac

SEQ ID NO: 78 - DNA, 25 bp, Artificial Sequence
  A synthetic oligonucleotide
caccttctacaatgagctgcgtgtg

SEQ ID NO: 79 - DNA, 22 bp, Artificial Sequence
  A synthetic oligonucleotide
tagataagcattataattccta

SEQ ID NO: 80 - DNA, 22 bp, Artificial Sequence
  A synthetic oligonucleotide
tatcacatatatttcaactcca

SEQ ID NO: 81 - DNA, 22 bp, Artificial Sequence
  A synthetic oligonucleotide
tacatttcttgtaactatcaca

SEQ ID NO: 82 - DNA, 22 bp, Artificial Sequence
  A synthetic oligonucleotide
tttcttctttgtatcgtatggc

SEQ ID NO: 83 - DNA, 22 bp, Artificial Sequence
  A synthetic oligonucleotide
tatttctttaagatgttgtgca

SEQ ID NO: 84 - DNA, 22 bp, Artificial Sequence
  A synthetic oligonucleotide
ttgaacatcagagagaaccagg

SEQ ID NO: 85 - DNA, 22 bp, Artificial Sequence
  A synthetic oligonucleotide
ttctaaagagcaatggttgctg

SEQ ID NO: 86 - DNA, 22 bp, Artificial Sequence
  A synthetic oligonucleotide
tattattatagtaacaggagca

SEQ ID NO: 87 - DNA, 22 bp, Artificial Sequence
  A synthetic oligonucleotide
ttgatcatcaattccaaacctc

SEQ ID NO: 88 - DNA, 23 bp, Artificial Sequence
  A synthetic oligonucleotide
tcatctggcatcaacataacagg

SEQ ID NO: 89 - DNA, 23 bp, Artificial Sequence
  A synthetic oligonucleotide
gaataggctttgaagtcatctgg

SEQ ID NO: 90 - DNA, 23 bp, Artificial Sequence
  A synthetic oligonucleotide
tcatctggcattagcatgacagg

SEQ ID NO: 91 - DNA, 23 bp, Artificial Sequence
  A synthetic oligonucleotide
gtccaccttgatcttgctgtagg

SEQ ID NO: 92 - DNA, 23 bp, Artificial Sequence
  A synthetic oligonucleotide
atctggcagcagcatcaccgggg

SEQ ID NO: 93 - protein, 7 aa, Homo sapiens
VMLLPDD

SEQ ID NO: 94 - protein, 7 aa, Homo sapiens
FHRENLP

SEQ ID NO: 95 - protein, 7 aa, Homo sapiens
EIFLPNN

SEQ ID NO: 96 - protein, 7 aa, Artificial Sequence
  A synthetic polypeptide
VMLXPDD